(19) United States
(12) Patent Application Publication
(10) Pub. No.: US 2016/0101106 A1
(43) Pub. Date: Apr. 14, 2016

(54) METHODS FOR TREATING HCV INFECTION

(71) Applicant: Inhibikase Therpeutics, Inc., Atlanta, GA (US)

(72) Inventor: Milton H Werner, Marietta, GA (US)

(73) Assignee: Inhibikase Therapeutics, Inc., Atlanta, GA (US)

(21) Appl. No.: 14/889,804

(22) PCT Filed: May 6, 2014
(86) PCT No.: PCT/US14/36877

§ 371 Date: Nov. 6, 2015

Related U.S. Application Data
(60) Provisional application No. 61/820,479, filed on May 7, 2013.

(51) Int. Cl. A61K 31/517; A61K 31/44; A61K 31/506; A61K 31/7072; A61K 31/4178; A61K 31/437; A61K 31/4439

(52) U.S. Cl. CPC A61K 31/517; A61K 31/437; A61K 31/44; A61K 31/4439; A61K 31/7072; A61K 31/4178; A61K 31/506

(57) ABSTRACT

The present invention provides methods of treating HCV infection in subjects (e.g., mammals), comprising administering to a subject in need thereof an effective amount of a RAF kinase inhibitor or a prodrug thereof. The present invention also provides methods of treating HCV infection, comprising administering to a subject in need thereof an effective amount of a RAF kinase inhibitor conjointly with at least one additional therapeutic agent (e.g., an antiviral agent).

### CROSS-REFERENCE TO RELATED APPLICATIONS

This application claims the benefit of and priority to U.S. Provisional Patent Application Ser. No. 61/820,479 filed 7 May 2013, which is hereby incorporated herein by reference in its entirety.

### BACKGROUND OF THE INVENTION

Hepatitis C virus (HCV) infection is a major global health problem with an estimated 150-200 million people having been infected worldwide. According to the World Health Organization, 3 to 4 million new infections occur each year. The onset of HCV infection is often insidious, with anorexia, vague abdominal discomfort, nausea and vomiting, fever and fatigue, progressing to jaundice in about 25% of patients. Of those exposed to HCV, about 40% recover fully whether they develop symptoms or not. However, the majority of HCV-infected individuals develop chronic infection, which often leads to serious liver disease, including fibrosis and steatosis. About 20% of patients with chronic HCV infection develop liver cirrhosis, of which up to 20% develop liver cancer.

Chronic HCV infection is the leading cause for liver transplantations. Unfortunately, liver transplantation is not a cure for hepatitis C; viral recurrence is an invariable problem and leading cause of graft loss. No vaccine protecting against HCV is available to date partially because the virus comes in many forms and constantly mutates leading to “swarms” of closely related viral genomic sequences (often referred to as quasi-species). The rationales for treatment of chronic hepatitis are to reduce inflammation, preventing progression to fibrosis. Current therapies include administration of ribavirin and/or interferon-alpha (IFN-α), two non-specific anti-viral agents. Combination therapy results in better treatment responses than mono therapy. For example, using a combination treatment of pegylated IFN-α and ribavirin, persistent clearance is achieved in about 50% patients with chronic hepatitis C. However, a large number of patients have contraindications to one of the components of the combination, cannot tolerate the treatment, do not respond to IFN therapy at all, or experience a relapse when administration is stopped. In addition to limited efficacy and substantial side effects such as neutropenia, haemolytic anemia and severe depression, current antiviral therapies are also characterized by high cost.

Until recently, the development of more effective therapeutics to combat HCV infection has been hampered by the lack of a cell culture system supporting HCV replication. Robust production of infectious HCV in cell culture has now been achieved using a unique HCV genome derived from the blood of a Japanese patient with fulminant hepatitis C (JFH-I). The ability of the JFH-I strain of HCV to release infectious particles in cell culture (HCVcc) and the development of retroviral HCV pseudoparticles (HCVpp) have allowed the complete viral life cycle to be explored. This, in turn, has led to the development of new antiviral agents targeting HCV protein processing and replication. However, many of these agents have proved to be toxic and highly susceptible to the development of viral resistance, suggesting that a different strategy is needed for the treatment of HCV infection. Moreover, given the health risks imposed on the public and the economic harms imposed on society by HCV infection, there is a pressing need for additional methods and agents for preventing and treatment of HCV infection and HCV-related disorders and diseases.

### SUMMARY OF THE INVENTION

The present invention relates to methods for treating HCV infection in a subject (e.g., a mammal) in need thereof. According to the present invention, there is provided a method of treating HCV infection comprising the step of administering to a subject in need thereof an effective amount of a compound that inhibits one or more RAF kinases.

In some embodiments, the compound suitable for use in the methods of the present invention is an inhibitor of one or more RAF kinases selected from A-RAF, B-RAF and C-RAF. In certain embodiments of the invention, the RAF inhibitor is selected from compounds described and referred to in U.S. Pat. Nos. 7,235,576 and 7,351,834, and International Patent Application Publications WO 02/24680, WO 2006/024834, WO 2008/147782, and WO 2009/117080 (all of which are incorporated herein by reference for their disclosure of RAF inhibitors). In certain embodiments of the invention, the RAF inhibitor is selected from sorafenib, CEP32496, Raf-265, regorafenib, SB-590885, and AZ-628 (structures shown below).

In accordance with another aspect of the present invention, there is provided a method for treating HCV infection in a subject in need thereof, the method comprising administering to said subject an RAF inhibitor or a pharmaceutical acceptable salt thereof conjointly with at least one additional therapeutic agent. In some embodiments, the additional therapeutic agent is an antiviral agent. In some embodiments, the additional therapeutic agent is a current, late stage or marketed direct-acting antiviral (DAA) agent that may be useful in treating HCV infection. Suitable antiviral agents include, but are not limited to, HCV protease inhibitors and HCV polymerase inhibitors (such as NS3/4A protease inhibitors and RNA-dependent RNA polymerase (NS5B) inhibitors), agents targeting host cell activities involved in HCV replication and inosine monophosphate dehydrogenase (IMPDH) inhibitors. In certain embodiments, the additional therapeutic agent is a DAA selected from Telaprevir (Vertex), Boceprevir (Merck), TMC435 (Tibotec), Danoprevir (Genentech/Roche), Vaniprevir (Merck), BI201335 (Boehringer-Ingelheim), Narlaprevir (Merck), BMS-650032 (Bristol-Myers Squibb), ABT-450 (Abbott), GS-9451 (Gilead), GS-9256 (Gilead), MK-5172 (Merck), RG7128 (Genentech/Roche), IDX184 (Idenix), PSI-7977 or GS-7977 (Pharmasset), PSI-938 (Pharmasset), Tegobuvir (Gilead), Filibuvir or PF-00868554 (Pfizer), ANA598 (Anadys), BI207127 (Boehringer-Ingelheim), ABT-333 (Abbott), VX-222 (Vertex), BMS-790052, Alisporivir (Novartis), and SCY-465 (Scynexis). In certain embodiments, the additional therapeutic agent is GS-7977 or BMS-790052 (structures shown below). In one embodiment, the method of the present disclosure comprises administering to a subject in need thereof a therapeutically effective amount of AZ-628 conjointly with GS-7977 or BMS-790052.

### BRIEF DESCRIPTION OF THE FIGURES

FIG. 1 shows pan-RAF kinase inhibitors and their potential targets in the Kit/RAF/MEK/ERK pathway. The tested inhibitors are shown in bold.

FIG. 2 shows the antiviral potency of AZ-628 against HCVpp, Genotype 1b, a virus-like particle enabling measurement of the ability of a Raf kinase inhibitor to block HCV entry into cultured human hepatocytes.

FIG. 3 shows the antiviral potency of AZ-628 against HCVcc, Genotype 2a, a replication competent, fully infectious form of hepatitis C virus.

FIG. 4 shows synergistic antiviral potency measured with AZ-628 and the direct-acting antiviral GS7977/Sobosuvir, a nucleotide analog that inhibits the viral RNA-dependent RNA polymerase of HCV. The three-dimensional plot displays the synergy volume data at the 95% confidence interval.

FIG. 5 shows synergistic antiviral potency against measured with AZ-628 and the direct-acting antiviral BMS-790052/Daclatasvir, an inhibitor of the non-structural protein NS5a, which plays a vital role in HCV genomic replication in the host. The three-dimensional plot displays the synergy volume data at the 95% confidence interval.

### DETAILED DESCRIPTION OF THE INVENTION

### Definitions

Unless otherwise defined herein, scientific and technical terms used in this application shall have the meanings that are commonly understood by those of ordinary skill in the art. Generally, nomenclature used in connection with, and techniques of, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present invention are generally performed, unless otherwise indicated, according to any suitable method, including conventional methods well known in the art and as described in various general and more specific references that are cited and discussed throughout this specification. See, e.g. Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); Gilbert et al., “Developmental Biology, 6th ed.”, Sinauer Associates, Inc., Sunderland, Ma. (2000).

Chemistry terms used herein are used according to conventional usage in the art, as exemplified by “The McGraw-Hill Dictionary of Chemical Terms”, Parker S., Ed., McGraw-Hill, San Francisco, Calif. (1985).

All of the publications, patents and published patent applications referred to in this application are specifically incorporated by reference herein. In case of conflict, the present specification, including its specific definitions, will control.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

The term “including” is used to mean “including but not limited to”, “Including” and “including but not limited to” are used interchangeably.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents which are known with respect to structure, and those which are not known with respect to structure. The RAF kinase inhibitory activity of such agents may render them suitable as “therapeutic agents” in the methods of this invention.

A “patient”, “subject”, or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Hepatitis C virus” or “HCV” is a small (55-65 nm in size), enveloped, positive-sense single-stranded RNA virus of the family Flaviviridae. HCV strains are usually classified into six genotypes and numerous subtypes on the basis of phylogenetic clustering. The methods of the present invention can be used to treat HCV infection and HCV-related disorders and diseases caused by any strain of the hepatitis C virus.

“HCV infection” as used herein refers to a process during which HCV particles bind to the cell surfaces of host cells, enter the host cells, proliferate in the host cells, and then are released outside the cells.

“Treating” a condition or patient refers to taking steps to obtain beneficial or desired results, including clinical results. Beneficial or desired clinical results include, but are not limited to, alleviation or amelioration of one or more symptoms associated with HCV-infection; delay or slowing of that infection; amelioration, palliation or stabilization of that infection. The present invention provides a method for treating HCV infection by using RAF kinase inhibitors. In some embodiments, the antiviral activity of such RAF kinase inhibitors is due to their ability to block the HCV viral entry into the host cells. In some embodiments, the antiviral activity of such RAF kinase inhibitors is due to their ability to inhibit viral propagation, reproduction, and/or replication. In some embodiments, the antiviral activity of such RAF kinase inhibitors is due to their ability to block the viral release.

“Administering” or “administration of” a substance, a compound or an agent to a subject can be carried out using any sultable method, such as one of a variety of methods known to those skilled in the art. For example, a compound or an agent can be administered, intravenously, arterially, intradermally, intramuscularly, intraperitoneally, intravenously, subcutancously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

Appropriate methods of administering a substance, a compound or an agent to a subject will also depend, for example, on the age of the subject, whether the subject is active or inactive at the time of administering, whether the subject is infected at the time of administering, the extent of the infection, and the chemical and biological properties of the compound or agent (e.g. solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. Thus, an individual who receives such treatment can benefit from a combined effect of different therapeutic compounds.

A “therapeutically effective amount” of a drug or agent is an amount of a drug or an agent that, when administered to a subject will have the intended therapeutic effect, e.g., treating HCV infection. The full therapeutic effect does not necessarily occur by administration of one dose, and may occur only after administration of a series of doses. Thus, a therapeutically effective amount may be administered in one or more doses. The precise effective amount needed for a subject will depend upon, for example, the subject's size, health and age, the nature and extent of the infection, and the therapeutics or combination of therapeutics selected for administration, and the mode of administration. The skilled worker can readily determine the effective amount for a given situation by routine experimentation.

“RAF family of kinases”, “RAF proteins”, or “RAF kinases”, as used herein interchangeably, include a set of three isozymes, A-RAF, B-RAF, and C-RAF. This family of kinases has emerged in the past several years as a promising target for protein-directed therapies. A-RAF, B-RAF, and C-RAF are protein-serine/threonine kinases that are related to retroviral oncogenes discovered in 1983. The murine sarcoma virus 3611 enhances fibrosarcoma induction in newborn MSF/N mice, and the name RAF corresponds to rapidly accelerated fibrosarcoma. RAF-1, which was discovered in 1985, is now called C-RAF. A-RAF was discovered in 1986, and B-RAF was discovered in 1988. B-RAF residue numbering changed in 2004 owing to a prior DNA sequencing error. Residues after position 32, in the original version, were one number short of their actual position.

The RAF proteins are central components of the mitogen-activated protein kinase (MAPK) pathway that regulates cell proliferation. Elimination of RAF function is predicted to be an effective treatment for the many cancers initiating with EGFR and Ras lesions. In particular, the core MAPK pathway is deemed as one of the most common sources of oncogenic lesions in cancer. Overexpression or mutation of members of the epidermal growth factor (EGFR) protein family leads to numerous cancers, including pancreatic, lung (adenocarcinoma and non-small cell lung cancer (NSCLC)), head and neck squamous cell cancer, colorectal, glioblastoma, and (for EGFR2/HER2/NEU/ERBB2) breast cancer. Increased expression and/or mutation-based activation of EGFR hyperactivates its downstream effector, Ras. Furthermore, Ras proteins are mutated, resulting in constitutive activation, in a high percentage of pancreatic, colon, and papillary thyroid cancers, and are also found in other cancers such as NSCLC and others. These changes in EGFR and Ras lead to a greatly enhanced level of Ras-dependent Raf activation, which in turn communicates signals downstream to MEK1/2 and the MAPKs ERK1 and ERK2. Although Ras has other important direct effectors in tumor promotion, including phosphoinositol-3-kinase (PI3K) and RalGDS the Raf>MEK>ERK signaling axis is essential for oncogenesis, based on validation in many systems.

“Inhibitor of RAF kinases”, “RAF kinase inhibitor” or “RAF inhibitor” refers to any agent, substance or compound that reduces the activities of one or more RAF kinases. A substance, or a compound or an agent is an inhibitor of RAF kinases even if it does not itself bind to a RAF kinase, as long as it causes, or affects the ability of, another compound or agent to bind RAF, and thus, deactivating the RAF kinase. In some embodiments, a RAF inhibitor inhibits only one RAF isozyme, such as A-RAF, B-RAF or C-RAF. In some embodiments, a RAF inhibitor inhibits more than one RAF isozyme. For instance, it can inhibit two types or even all three types of the isozymes. In some embodiments, a RAF inhibitor inhibits both B-RAF and C-RAF. In some embodiments, the RAF inhibitor is a class I RAF inhibitor, which has cellular selectivity for B-RAF mutations associated with melanoma, such as V600E. Such class I inhibitors bind wild-type B-RAF with equal proficiency to mutant protein in purified form, but selectively target the mutant protein when present inside cells. Class I RAF inhibitors include, but are not limited to, PLX4032 and GSK2318436. In some embodiments, the RAF inhibitor is a class II RAF inhibitor, which does not discriminate mutant B-RAF from their wild-type counterpart. Class II RAF inhibitors include, but are not limited to, sorafenib, RAF-265 and AZ-628. Inhibitors of RAF kinases, as used herein, include pharmaceutically acceptable salts, derivatives, analogs, prodrugs, and polymorphs of the inhibitors thereof.

“Pharmaceutically acceptable salts” is used/herein to refer to an agent or a compound according to the invention that is a therapeutically active, non-toxic base and acid salt form of the compounds. The acid addition salt form of a compound that occurs in its free form as a base can be obtained by treating said free base form with an appropriate acid such as an inorganic acid, for example, a hydrohalic such as hydrochloric or hydrobromic, sulfuric, nitric, phosphoric and the like; or an organic acid, such as, for example, acetic, hydroxyacetic, propanoic, lactic, pyruvic, malonic, succinic, maleic, fumaric, malic, tartaric, citric, methanesulfonic, ethanesulfonic, benzenesulfonic, p-toluenesulfonic, cyclic, salicylic, p-aminosalicylic, pamoic and the like. See, e.g., WO 01/062726.

Compounds containing acidic protons may be converted into their therapeutically active, non-toxic base addition salt form, e.g. metal or amine salts, by treatment with appropriate organic and inorganic bases. Appropriate base salt forms include, for example, ammonium salts, alkali and earth alkaline metal salts, e.g. lithium, sodium, potassium, magnesium, calcium salts and the like, salts with organic bases, e.g. N-methyl-D-glucamine, hydrabamine salts, and salts with amino acids such as, for example, arginine, lysine and the like. Conversely, said salt forms can be converted into the free forms by treatment with an appropriate base or acid. Compounds and their salts can be in the form of a solvate, which is included within the scope of the present invention. Such solvates include for example hydrates, alcoholates and the like. See, e.g., WO 01/062726.

The present invention includes within its scope, prodrugs, analogs, derivatives and polymorphs of the RAF inhibitors of the invention. In general, such prodrugs will be functional derivatives of a compound of the invention which are readily convertible in vivo into the compound from which it is notionally derived. Conventional procedures for the selection and preparation of suitable prodrugs are described, for example, in “Design of Prodrugs” Ed. H. Bundgaard, Elsevier, 1985, the contents of which are hereby incorporated by reference herein in their entirety. For example, a common method for making a prodrug is to select moieties which are hydrolyzed or metabolized under physiological conditions to provide the desired compound or agent. In other embodiments, the prodrug is converted by an enzymatic activity of the host animal to an inhibitor of RAF kinases.

“Analog” is used herein to refer to a compound which functionally resembles another chemical entity, but does not share the identical chemical structure. For example, an analog is sufficiently similar to a base or parent compound such that it can substitute for the base compound in therapeutic applications, despite minor structural differences.

“Derivative” is used herein to refer to the chemical modification of a compound. Chemical modifications of a compound can include, for example, replacement of hydrogen by an alkyl, acyl, or amino group. Many other modifications are also possible.

As used herein, the term “polymorph” refers to different crystalline forms of the same compound and other solid state molecular forms including pseudo-polymorphs, such as hydrates (e.g., bound water present in the crystalline structure) and solvates (e.g., bound solvents other than water) of the same compound. Different crystalline polymorphs have different crystal structures due to a different packing of the molecules in the lattice. This results in a different crystal symmetry and/or unit cell parameters which directly influences its physical properties such as the X-ray diffraction characteristics of crystals or powders. A different polymorph, for example, will in general diffract at a different set of angles and will give different values for the intensities. Therefore X-ray powder diffraction can be used to identify different polymorphs, or a solid form that comprises more than one polymorph, in a reproducible and reliable way. Crystalline polymorphic forms are of interest to the pharmaceutical industry and especially to those involved in the development of suitable dosage forms. If the polymorphic form is not held constant during clinical or stability studies, the exact dosage form used or studied may not be comparable from one lot to another. It is also desirable to have processes for producing a compound with the selected polymorphic form in high purity when the compound is used in clinical studies or commercial products since impurities present may produce undesired toxicological effects. Certain polymorphic forms may exhibit enhanced thermodynamic stability or may be more readily manufactured in high purity in large quantities, and thus are more suitable for inclusion in pharmaceutical formulations. Certain polymorphs may display other advantageous physical properties such as lack of hygroscopic tendencies, improved solubility, and enhanced rates of dissolution due to different lattice energies.

Many of the compounds useful in the methods of this invention have at least one stereogenic center in their structure. This stereogenic center may be present in a R or a S configuration, said R and S notation is used in correspondence with the rules described in Pure Appl. Chem. (1976), 45, 11-30. The invention also relates to all stereoisomeric forms such as enantiomeric and diastereoisomeric forms of the compounds or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the invention includes both mixtures and separated individual isomers. Some of the compounds may also exist in tautomeric forms. Such forms although not explicitly indicated in the above formula are intended to be included within the scope of the present invention. With respect to the methods and compositions of the present invention, reference to a compound or compounds is intended to encompass that compound in each of its possible isomeric forms and mixtures thereof unless the particular isomeric form is referred to specifically. See, e.g., WO 01/062726.

### RAF Kinase Inhibitors

Among the RAF inhibitors useful for the methods of this invention are those compounds or agents referred to in:

(i) PCT Application Publication WO 2006/024834, which is incorporated herein by reference:

A compound of formula A-I:

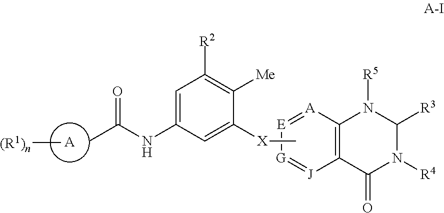

wherein;
Ring A is carbocyclyl or heterocyclyl; wherein if said heterocyclyl contains an —NH-moiety that nitrogen may be optionally substituted by a group selected from R⁶;
R¹ is a substituent on carbon and is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl; N-(C₁₋₆alkyl)sulphamoyl, N,N-(C₁₋₆alkyl)₂sulphamoyl, N-(C₁₋₆alkoxy)sulphamoyl, N-(C₁₋₆alkyl)-N-(C₁₋₆alkoxy)sulphamoyl, C₁₋₆alkylsulphonylamino, carbocycyl-R⁷- or heterocyclyl-R⁸-; wherein R¹ may be optionally substituted on carbon by one or more R⁹; and wherein if said heterocyclyl contains an —NH— moiety that nitrogen may be optionally substituted by a group selected from R¹⁰;
n is selected from 0-4; wherein the values of R¹ may be the same or different;
R² is selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₂₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl, N,N-(C₁₋₆alkyl)2sulphamoyl, C₁₋₆alkylsulphonylamino, carbocyclyl-R¹¹- or heterocyclyl-R¹²-; wherein R² may be optionally substituted on carbon by one or more R¹³; and wherein if said heterocyclyl contains an —NH— moiety that nitrogen may be optionally substituted by a group selected from R¹⁴;
X is NR¹⁵ or O;
one of A, E, G and J is C which is attached to X of formula A-I; the other three are independently selected from CR¹⁶ or N;
R³ and R¹⁶ are independently selected from hydrogen, halo, nitro, cyano, hydroxy, amino, carboxy, carbamoyl, mercapto, sulphamoyl, C₁₋₆alkyl, C₁₋₆alkenyl, C₁₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbarmoyl, C₁₋₆alkylS(O)ₐ wherein a is 0 to 2, C₁₋₆alkoxycarbonyl, N-(C₁₋₆alkyl)sulphamoyl, N,N-(C₁₋₆alkyl)₂sulphamoyl, C₁₋₆alkylsulphonylamino, carbocyclyl-R¹⁷- or heterocyclyl-R¹ wherein R³ and R¹⁶ independently of each other may be optionally substituted on carbon by one or more R¹⁹; and wherein if said heterocyclyl contains an —NH— moiety that nitrogen may be optionally substituted by a group selected from R²⁰;
R⁴, R⁵ and R¹⁵ are independently selected from hydrogen, C₁₋₆alkyl, C₁₋₆alkylsulphonyl, C₁₋₆alkoxycarbonyl, carbamoyl, carbocyclyl, heterocyclyl, N-(C₁₋₆alkyl)carbamoyl and N,N-(C₁₋₆alkyl)carbamoyl; wherein R⁴, R⁵ and R¹⁵ independently of each other may be optionally substituted on carbon by one or more R²¹;
the bond “ [symbol] ” between the —NR⁵- and —CR³- of formula A-I is either (i) a single bond wherein R⁵ is as defined above, or (ii) a double bond wherein R⁵ is absent;
R⁹, R¹³, R¹⁹ and R²¹ are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆lkyl)₂carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino, carbocyclyl-R²²- or heterocyclyl-R²³-; wherein R⁹, R¹³, R¹⁹ and R²¹ independently of each other may be optionally substituted on carbon by one or more R²⁴; and wherein if said heterocyclyl contains an —NH— moiety that nitrogen may be optionally substituted by a group selected from R²⁵;
R⁷, R⁸, R¹², R¹⁷, R¹⁸, R²² and R²³ are independently selected from a direct bond, —O—, —N(R²⁶)—, —C(O)—, —N(R²⁷)C(O)—, —C(O)N(R²⁸ )—, —S(O)ₛ—, —SO₂N(R²⁹)— or —N(R³⁰)SO₂—; wherein R²⁶, R²⁷, R²⁸, R²⁹ and R³⁰ is hydrogen, C₁₋₆alkoxycarbonyl or C₁₋₆alkyl and s is 0-2;
R⁶, R¹⁰, R¹⁴, R²⁰ and R²⁵ are independently selected from C₁₋₆alkyl, C₁₋₆alkylsulphonyl, C₁₋₆alkoxycarbonyl, carbamoyl, N-(C₁₋₆alkylcarbamoyl, N,N(C₁₋₆alkyl)carbamoyl, benzyl, benzyloxycarbonyl, benzoyl and phenylsulphonyl;
R²⁴ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl; or a pharmaceutically acceptable salt thereof.

In some embodiment, the compound has a structure of formula A-I:

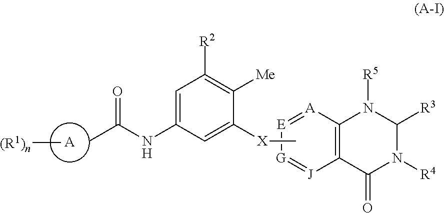

wherein:
Ring A is phenyl, thien-2-yl, thien-3-yl, pyrid-2-yl, pyrid-3-yl, pyrid-4-yl, thiazol-4-yl, isoxazol-3-yl, 1,3-benzodioxol-5-yl, fur-2-yl, 1-methylpyrazol-3-yl, 1-methylpyrazol-5-yl, 1-t-butylpyrazol-5-yl, indol-5-yl, indol-6-yl, 2,3-dihydrobenzofuran-7-yl, imidazo[1,2-α]pyridin-2-yl or pyrimidin-4-yl;
R¹ is a substituent on carbon and is selected from fluoro, chloro, bromo, hydroxy, cyano, sulphamoyl, methyl trifluoromethyl, cyclopropylaminomethyl, methylthiomethyl, mesylmethyl, dimethylaminomethyl, 1-(cyclopropyl)-1-hydroxymethyl, N-cyclopropyl-N-(t-butoxycarbonylaminomethyl, 1-methylpiperazin-4-ylmethyl, 1-hydroxy-1-cyclopropylethyl, 1-methyl-1-cyanoethyl, 2-methoxy-1,1-dimethylethyl, 1-carboxy-1-methylethyl, 1,1-difluoroethyl, 2-(dimethylamino)-1,1-dimethyl-2-oxoethyl, 3-(dimethylamino)propyl, 1,1-dimethylpropyl, t-butyl, methoxy, N-methylcarbamoylmethoxy, 2-(dimethylamino)ethoxy, 2-(pyrrolidin-1-yl)ethoxy, 2-(methoxy)ethoxy, 2-(1-methylpyrrolidin-2-yl)ethoxy, 2-(piperidin-1-yl)ethoxy, 2-(azepan-1-yl)ethoxy, 2-(morpholino)ethoxy, 3-(1-methylpiperazin-4-yl)propoxy, methoxycarbonyl, morpholinocarbonyl, N,N-dimethylsulphamoyl, N-(2,3-dihydroxypropyl)-N-methylsulphamoyl, N-(methyl)-N-(methoxy)sulphamoyl, 1-methylpiperidin-4-yloxy, N,N-dimethylcarbamoyl, cyclopropyl, piperidin-1-yl morpholino, 1-cyclopropylethenyl, 3-(4-methylpiperazin-1-yl)prop-1-yn-1-yl, 3,3-dimethylbut-1-yn-1-yl, cyclopropylethynyl, 3-hydroxy-3-methylbut-1-yn-1-yl, 1,1-dimethylprop-2-yn-1-yl, 3-(dimemylamimo)prop-1-yn-1-yl, mesyl, cyclopropylaminosulphonyl, azetidin-1-yl-sulphonyl, morpholinosulphonyl, tetrahydrofur-2-ylmethylaminosulphonyl, 2-(hydroxymethyl)piperidin-1-ylsulphonyl, 3-(hydroxymethyl)piperidin-1-ylsulphonyl or 4-(hydroxymethyl)piperidin-1-ylsulphonyl;
n is selected from. 0-2; wherein the values of R¹ may he the same or different;
R² is hydrogen;
X is NR¹⁵ or O;
one of A, E, G and J is C which is attached, to X of formula A-I; the other three are all CR¹⁶ or two are CR¹⁶ and one is N;
R³ is selected from hydrogen, methyl, N-(2-hydroxyethyl)amino, N,N-dimemylamino or methylthio;
R⁴ is selected from hydrogen, methyl, 1-methylpiperidin-3-ylmethyl, cyclopropylmethyl, 2,2-dimethyl-1,3-dioxolan-4-ylmethyl, piperidin-4-ylmethyl, 1-benzyloxycarbonylpipidin-4-ylmethyl, ethyl, 2-hydroxyethyl, 3-aminopropyl, 3-(t-butoxycarbonylamino)propyl, 3-morpholinopropyl, 2,3-dihydroxypropyl and cyclopropyl;
the bond “ [symbol] ” between the —NR⁵— and —CR³— of formula A-I is a double bond wherein R⁵ is absent; and
R¹⁵ is selected from hydrogen, methyl or cyclopropylmethyl;
R¹⁶ is hydrogen;
or a pharmaceutically acceptable salt thereof.

In some embodiments, the compound is selected from:
3-(1,1-dimethylprop-2-yn-1-yl)-N-{4-methyl-3-[(3-methyl-4-oxo-3,4-dihydroquinazolin-6-yl)amino]phenyl}benzamide;
3-(1-cyano1-methylethyl)-N-{4-methyl-3-[(3-methyl-4-oxo-3,4-dihydroquinazolin-6-yl)amino]phenyl}benzamide;
3-(1-cyano-1-methylethyl)-5-fluoro-N-{4-methyl-3-[(3-methyl-4-oxo-3,4-dihydroquinazolin-6-yl)amino]phenyl}benzamide;
3-(1-cyano-1-methylethyl)-5-[(dimethylamino)methyl]-N-{4-methyl-3-[3-methyl-4-oxo-3,4-dihydroquinazolin-6-yl)amino]phenyl}benzamide;
4-dimethylaminomethyl-N-[4-methyl-3-(3-methyl-4-oxo-3,4-dihydro-quinazolin-6-ylamino)-phenyl]-3-trifluoromethyl-benzamide;
2-(1-cyano-1-methylethyl)-N-14-methyl-3-[(3-methyl-4-oxo-3,4-dihydroquinazolin-6- yl)amino]phenyl} isonicotinamide;
3-(cyano-1-methylethyl)-2-fluoro-N-{4-methyl-3-[(3-methyl-4-oxo-3,4-dihydroquinazolin-6-yl)amino]phenyl}benzamide;
N-(3-{[3-(3-aminopropyl)-4-oxo-3,4-dihydroquinazolin-6-yl]amino}-4-methylphenyl)-3-(1-cyano-1-methylethyl)benzamide;
3-{[methoxy(methyl)amino]sulfonyl}-N-{4-methyl-3-[(3-methyl-4-oxo-3,4-dihydroquinazolin-6-yl)amino]phenyl}benzamide; and
3-tert-butyl-N-{4-methyl-3-[3-methyl-4-oxo-3,4-dihydroquinazolin-6-yl)amino]phenyl}benzamide;
or a pharmaceutically acceptable salt thereof.

In some embodiments, the compound is AZ-628:

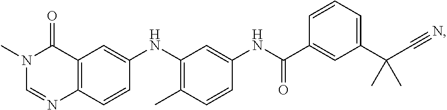 [SMILES: Cc1ccc(NC(=O)c2cccc(C(C)(C)C#N)c2)cc1Nc1ccc2ncn(C)c(=O)c2c1]

or a pharmaceutically acceptable salt thereof.

(ii) U.S. Pat. Nos. 7,235,576 and 7,351,834, both of which are incorporated herein by reference:

A compound of formula B-I:

A-D-B (B-I)

wherein D is —NH—C(O)—NH—,
A is a substituted moiety of up to 40 carbon atoms of the formula: -L-(M-L¹)_{q}, where L is a 5 or 6 membered cyclic structure bound directly to D, L¹ comprises a substituted cyclic moiety having at least 5 members, M is a bridging group having at least one atom, q is an integer of from 1-3; and each cyclic structure of L and L¹ contains 0-4 members of the group consisting of nitrogen, oxygen and sulfur, and
B is a substituted or unsubstituted, up to tricyclic aryl or heteroaryl moiety of up to 30 carbon atoms with at least one 6-member cyclic structure bound directly to D containing 0-4 members of the group consisting of nitrogen, oxygen and sulfur,
wherein L¹ is substituted by at least one substituent selected from the group consisting of —SO₂Rₓ, —C(O) Rₓ and —C(NR_{y})R_{z},
R_{y} is hydrogen or a carbon based moiety of up to 24 carbon atoms optionally containing heteroatoms selected from N, S and O and optionally halosubstituted, up to per halo,
R_{z} is hydrogen or a carbon based moiety of up to 30 carbon atoms optionally containing heteroatoms selected from N, S and O and optionally substituted by halogen, hydroxy and carbon based substituents of up to 24 carbon atoms, which optionally contain heteroatoms selected from N, S and O and are optionally substituted by halogen;
Rₓ is R_{z} or NRₐR_{b} where Rₐ and R_{b} are
   a) independently hydrogen,
      a carbon, based moiety of up to 30 carbon atoms optionally containing heteroatoms selected from N, S and O and optionally substituted by halogen, hydroxy and carbon based substituents of up to 24 carbon atoms, which optionally contain heteroatoms selected from N, S and O and are optionally substituted by halogen, or
      —OSi(R_{f})₃ where Rᵣ is hydrogen or a carbon based moiety of up to 24 carbon atoms optionally containing heteroatoms selected from N, S and O and optionally substituted by halogen, hydroxy and carbon based substituents of up to 24 carbon atoms, which optionally contain heteroatoms selected from N, S and O and are optionally substituted by halogen; or
   b) Rₐ and R_{b} together form a 5-7 member heterocyclic structure of 1-3 heteroatoms selected from N, S and O, or a substituted 5-7 member heterocyclic structure of 1-3 heteroatoms selected from N, S and O substituted by halogen, hydroxy or carbon based substituents of up to 24 carbon atoms, which optionally contain heteroatoms selected from N, S and O and are optionally substituted by halogen; or
   c) one of Rₐ or R_{b} is —C(O)—, a C₁-C₅ divalent alkylene group or a substituted C₃-C₅ divalent alkylene group bound to the moiety L to form a cyclic structure with at least 5 members, wherein the substituents of the substituted C₁-C₅ divalent alkylene group are selected from the group consisting of halogen, hydroxyl, and carbon based substituents of up to 24 carbon atoms, which optionally contain heteroatoms selected from N, S and O and are optionally substituted by halogen;
where B is substituted, L is substituted of L¹ is additionally substituted, the substituents are selected from the group consisting of halogen, up to per-halo, and Wn, where n is 0-3;
wherein each W is independently selected from the group consisting of —CN, —CO₂R⁷, —C(O)NR⁷R⁷, —C(O)—R⁷, —NO₂, —OR⁷, —SR⁷, —NR⁷R⁷, —NR⁷C(O)OR⁷, —NR⁷C(O)R⁷, —Q—Ar, and carbon based moieties of up to 24 carbon atoms, optionally containing heteroatoms selected from N, S and O and optionally substituted by one or more substituents independently selected from the group consisting of —CN, —CO₂R⁷, —C(O)R⁷, —C(O)NR⁷R⁷, —OR⁷, —SR⁷, —NR⁷R⁷, —NO₂, —NR⁷C(O)R⁷, —NR⁷C(O)OR⁷ and halogen up to per-halo; with each R⁷ independently selected from H or a carbon based moiety of up to 24 carbon atoms, optionally containing heteroatoms selected from N, S and O and optionally substituted by halogen;
wherein Q is —O—, —S—, —N(R⁷)—, —(CH₂)ₘ, —C(O)—, —CH(OH)—, —(CH₂)ₘO—, —(CH₂)ₘS—, —(CH₂)ₘN(R⁷)—, —O(CH₂)ₘ—CHX^{a}—, —CX^{a}₂—, —S—(CH₂)ₘ— and —N(R⁷)(CH₂)ₘ—, where m=1−3, and X^{a} is halogen; and
Ar is a 5- or 6-member aromatic structure containing 0-2 members selected from the group consisting of nitrogen, oxygen and sulfur, which is optionally substituted by halogen, up to per-halo, and optionally substituted by Zₙ₁, wherein n1 is 0 to 3 and each Z is independently selected from the group consisting of —CN, —CO₂R⁷, —C(O)R⁷, —C(O)NR⁷R⁷, —NO₂, —OR⁷, —SR⁷—NR⁷R⁷, —NR⁷C(O)OR⁷, —NR⁷C(O)R⁷, and a carbon based moiety of up to 24 carbon atoms, optionally containing heteroatoms selected from N, S and O and optionally substituted by one or more substituents selected from the group consisting of —CN, —CO₂R⁷, —COR⁷, —C(O)NR⁷R⁷, —OR⁷, —SR⁷, —NO₂,NR⁷R⁷, —NR⁷C(O)R⁷, and —NR⁷C(O)OR⁷, with R⁷ as defined above.

In formula B-I, suitable hetaryl groups include, but are not limited to, 5-12 carbon-atom aromatic rings or ring systems containing 1-3 rings, at least one of which is aromatic, in which one or more, e.g., 1-4 carbon atoms in one or more of the rings can be replaced by oxygen, nitrogen or sulfur atoms. Each ring typically has 3-7 atoms. For example, B can be 2- or 3-furyl, 2- or 3-thienyl, 2- or 4-triazinyl, 1-, 2- or 3-pyrrolyl, 1-, 2-, 4- or 5-imidazolyl, 1-, 3-, 4- or 5-pyrazolyl, 2-, 4- or 5-oxazolyl 3-, 4, or 5-isoxazolyl, 2-, 4- or 5-thiazolyl, 3-, 4- or 5-isothiazolyl, 2-, 3- or 4-pyridyl, 2-, 4-, 5- or 6-pyrimidinyl 1,2,3-triazol-1-, -4- or -5-yl, 1,2,4-triazol-1-, -3- or -5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-oxadiazol-3- or -5-yl, 1,3,4-thiadiazol-2- or -5yl, 1,3,4-thiadiazol-3- or -5-yl, 1,2,3-thiadiazol-4- or -5-yl, 2-, 3-, 4-, 5- or 6-2H-thiopyranyl 2-, 3- or 4-4H-thiopyranyl 3- or 4-pyridazinyl, pyrazinyl, 2-, 3-, 4-, 5-, 6- or 7-benzofuryl, 2-, 3-, 4-, 5-, 6- or 7-benzothienyl, 1-, 2-, 3-, 4-, 5-, 6- or 7-indolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 1-, 3-, 4-, 5-, 6- or 7-benzothiazolyl, 2-, 4-, 5-, 6- or 7-benzisothiazolyl 2-, 4-, 5-, 6- or 7-benz-1,3-oxadiazolyl, 2-, 3-, 4-, 5-, 6-, 7- or 8-quinolinyl, 1-, 3-, 4-, 5-, 6-, 7-, 8-isoquinolinyl, 1-, 2-, 3-, 4- or 9-carbazolyl, 1-, 2-, 3-, 4-, 5-, 6-, 7-, 8- or 9-acridinyl, or 2-, 4-, 5-, 6-, 7- or 8-quinazolinyl, or additionally optionally substituted phenyl, 2- or 3-thienyl, 1,3,4-thiadiazolyl, 3-pyrryl, 3-pyrazolyl, 2-thiazolyl or 5-thiazolyl, etc. For example, B can be 4-methyl-phenyl, 5-methyl-2-thienyl, 4-methyl-2-thienyl 1-methyl-3-pyrryl, 1-methyl-3-pyrazolyl, 5-methyl-2-thiazolyl or 5-methyl-1,2,4-thiadiazol-2-yl.

Suitable alkyl groups and alkyl portions of groups, e.g., alkoxy, etc. throughout include methyl, ethyl, propyl, butyl, etc., including all straight-chain and branched isomers such as isopropyl, isobutyl, sec-butyl, tert-butyl, etc.

Suitable aryl groups which do not contain heteroatoms include, for example, phenyl and 1- and 2-naphthyl.

Suitable halogen groups include F, Cl, Br, and/or I, from one to per-substitution (i.e. all H atoms on a group replaced by a halogen atom) being possible where an alkyl group is substituted by halogen, mixed substitution of halogen atom types also being possible on a given moiety.

The methods of the invention relate to compounds of formula B-I as well as pharmaceutically acceptable salts of formula B-I. Suitable pharmaceutically acceptable salts are well known to those skilled in the art and include basic salts of inorganic and organic acids, such as hydrochloric acid, hydrobromic acid, sulfuric acid, phosphoric acid, methanesulphonic acid, trifluoromethane-sulfonic acid, benzenesulfonic acid, p-toluenesulfonic acid, 1-naphthalenesulfonic acid, 2-naphthalenesulfonic acid, acetic acid, trifluoroacetic acid, malic acid, tartaric acid, citric acid, lactic acid, oxalic acid, succinic acid, fumaric acid, maleic acid, benzolc acid, salicylic acid, phenylacetic acid, and mandelic acid. In addition, pharmaceutically acceptable salts include acid salts of inorganic bases, such as salts containing alkaline cations (e.g., Li⁺ Na⁺ or K⁺), alkaline earth cations (e.g., Mg⁺², Ca⁺² or Ba⁺²), the ammonium cation, as well as acid salts of organic bases, including aliphatic and aromatic substituted ammonium, and quaternary ammonium cations, such as those arising from proto-nation or peralkylation of triethylamine, N,N-diethylamine, N,N-dicyclohexylamine, lysine, pyridine, N,N-dimethylaminopyridine (DMAP), 1,4-diazabiclo[2.2.2] octane (DABCO), 1,5-diazabicyclo[4.3.0]non-5-ene(DBN) and 1,8-diazabicyclo[5.4.0]undec-7ene (DBU).

In some embodiments of the compound of formula B-I, the compound is selected from the group consisting of:
N-(5-tert-butyl-2-methoxy phenyl)-N′-(4-(4-methoxy-3-(N-methylcarbamoyl)phenoxy) phenyl)urea,
N-(2-methoxy-5-(trifluoromethyl)phenyl)-N′-(4-(2-(N-methylcarbamoyl)-4-pyridyloxy) phenyl)urea,
N-(4-chloro-3-(trifluoromethyl(phenyl)-N′-(4-(2-carbamoyl-4-pridyloxy)phenyl)urea,
N-(4-chloro-3-(trifluoromethyl)phenyl)-N′-(4-(2-methylcarbamoyl)-4-pyridyloxy) phenyl)urea, or
N-(2-methoxy-4-chloro-5-(trifluoromethyl)phenyl)-N′-(3-(2-(N-methylcarbamoyl)-4-pyridyloxy)phenyl)urea and their pharmaceutically acceptable safe.

In some embodiments, the compound has a structure of formula B-I:

A-D-B (B-I)

or a pharmaceutically acceptable salt thereof, wherein D is —NH—C(O) —NH—, A is substituted moiety of the formula: -L-M-L¹
wherein L is phenyl, optionally substituted by halogen, up to per-halo, and Wn, where n is 0-3;
wherein each W is independently selected from the group consisting of C₁-C₅ linear or branched alkyl, C₁-C₅ linear or branched haloalkyl up to perhaloalkyl and C₁-C₃ alkoxy L¹ is selected from pyridinyl substituted by —C(O)Rₓ, and
optionally substituted with 1-3 additional substituents independently selected from the group consisting of R⁷ and halogen;
wherein Rₓ is NRₐR_{b} and Rₐ and R_{b} are
A) independently
   a) hydrogen,
   b) C₁-C₁₀ alkyl,
   c) C₆ aryl,
   d) pyridinyl
   e) substituted C₁₋₁₀ alkyl,
   f) substituted C₆ aryl,
   g) substituted pyridinyl
   h)-phenylpiperazine(pyridinyl),
   i)-phenylmorpholinyl
   j)-ethylmorpholinyl,
   k)-ethylpiperidyl,
   l)-methyl pyrrolidinyl,
   m)-methyl terrahydrofuryl,
   or
   n) —C₂H₄NH(phenyl);
where when Rₐ and R_{b} are a substituted group, they are substituted by
   a) halogen up to per halo,
   b) hydroxy,
   c) —N(CH₃)₂,
   d) C₁-C₁₀ alkyl,
   e) C₁-C₁₀ alkoxy,
   f) halosubstituted C₁₋₆ alkyl, or
   g)—OSi(Pr-i)₃; or
B) Rₐ and R_{b} together form piperazine or a substituted piperazine with one or more substituents selected from the group consisting of
   a) halogen.
   b) hydroxy,
   c) C₁-C₁₀ alkyl,
   d) pyridinyl
   e) C₁-C₁₀ alkoxy,
   f) C₆ aryl,
   g) halo substituted C₆ aryl, and
   h) N-(4-acetylphenyl);
M is selected from the group consisting of oxygen and sulfur; and
B is phenyl, substituted with 1-3 substituents independently selected from the group consisting of halogen and R⁷,
and R⁷ is
   (a) C₁-C₆ linear or branched alkyl, optionally substituted with 1-3 halogen substituents; or
   (b) C₁-C₆ linear or branched alkoxy,

In certain embodiments, the compound has a structure of formula B-I:

A-D-B (B-I)

or a pharmaceutically acceptable salt thereof wherein
D is —NH—C(O)—NH—,
A is of the formula: -L-M-L¹, wherein
L is phenyl, optionally substituted with 1-3 substituents independently selected from the group consisting of C₁-C₅ linear or branched alkyl, C₁-C₅ linear or branched haloalkyl up to perhalo, C₁-C₃ alkoxy and halogen;
L¹ is pyridinyl, substituted by —C(O)Rₓ;
   wherein Rₓ is NRₐR_{b} and Rₐ and R_{b} are independently hydrogen,
   C₁-C₁₀ alkyl,
   C₆ aryl,
   pyridinyl, substituted C₁-C₁₀ alkyl,
   substituted C₆ aryl, or
   substituted pyridinyl,
   where Rₐ and R_{b} are a substituted group, they are substituted by halogen up to per halo; and
M is selected from the group consisting of oxygen and sulfur and
B is phenyl, substituted with 1-3 substituents independently selected from the group consisting of R⁷ and halogen;
and R⁷ is
   (a) C₁-C₆ linear or branched alkyl, optionally substituted with 1-3 halogen substituents;
   or
   (b) C₁-C₆ linear or branched alkoxy.

In some embodiments, the compound has the structure of formula B-I:

A-D-B (B-I)

or a pharmaceutically acceptable salt thereof, wherein
D is —NH—C(O) —NH—,
A is of the formula: -L-M-L¹,
L is phenyl,
M is —O—,
L¹ is pyridinyl substituted by —C(O)Rₓ,
   wherein Rₓ is NRₐR_{b} and Rₐ and R_{b} are independently hydrogen,
      C₁-C₁₀ alkyl,
      C₆ aryl,
      pyridinyl,
      substituted C₁₋₁₀ alkyl,
      substituted C₆ aryl, or
      substituted pyridinyl
   where Rₐ and R_{b} are a substituted group, they are substituted by halogen up to per halo, and
B is a phenyl group substituted by trifluoromethyl or tert-butyl, and optionally additional substituents selected from the group consisting of halogen up to per halo, and Wₙ where n is 0-3, and each w is independently selected from the group consisting of
   C₋C₁₀ alkyl,
   C₁-C₁₀ alkoxy,
   C₆ aryl,
   pyridinyl,
   and substituted C₁-C₁₀ alkyl, substituted by one or more substituents independently selected from the group consisting of halogen up to per halo.

In some embodiments, the RAF inhibitor is sorafenib:

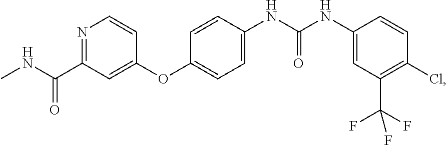 [SMILES: CNC(=O)c1cc(Oc2ccc(NC(=O)Nc3ccc(Cl)c(C(F)(F)F)c3)cc2)ccn1]

or a pharmaceutically acceptable salt thereof,

In some embodiments, the RAF inhibitor is regorafenib:

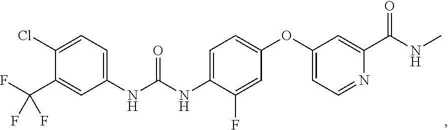 [SMILES: CNC(=O)c1cc(Oc2ccc(NC(=O)Nc3ccc(Cl)c(C(F)(F)F)c3)c(F)c2)ccn1]

or a pharmaceutically acceptable salt thereof.

(iii) PCT Application Publication WO 2009/117080, which is incorporated herein by reference:

A compound having formula C-I:

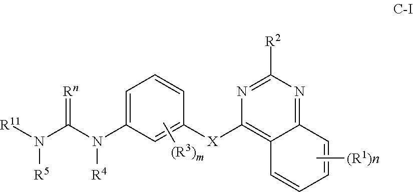

or a pharmaceutically acceptable salt or prodrug thereof, wherein
X is O or S(O)ₜ;
R^{a} is O or S;
each R¹ is independently selected from (i) or (ii) below:
   (i) each R¹ is independently selected from a group consisting of halo, nitro, alkyl, alkenyl, alkynyl, cycloalkyl, cycloalkylalkyl, hetetocyclyl, heterocyclylalkyl, aryl, aralkyl, heteroaryl, heteroaralkyl, —R⁶OR⁷, —R⁶SR⁷, —R⁶S(O)₁R⁸, —R⁶N(R⁷)₂, —R⁶OR⁹OR⁷, —R⁶OR⁹SR⁷, —R⁶OR⁹S(O)ₜR⁸, —R⁶OR⁹S(O)ₜN(R⁷)₂. —R⁶OR⁹N(R⁷)₂, —R⁶SR⁹OR⁷, —R⁶SR⁹SR⁷, —R⁶SR⁹N(R⁷)₂, —R⁶N(R⁷)R⁹N(R⁷)₂, —R⁶N(R⁷)R⁹OR⁷, —R⁶N(R⁷)R⁹SR⁷, —R⁶CN, —R⁶C(O)R⁷, —R⁶C(O)OR⁷, —R⁶C(O)OR⁹OR⁷, —R⁶C(O)N(R⁷)₂, —R⁶C(O)N(R⁷)OR⁷; —R⁶C(NR⁷)N(R⁷)₂, —R⁶C(O)N(R⁷)R⁹N(R⁷)₂, —R⁶C(O)N(R⁷)R⁹OR⁷, —R⁶C(O)N(R⁷)R⁹SR⁷, —R⁶C(O)SR⁸, —R⁶S(O)ₜOR⁷, —R⁶S(O)ₜN(R⁷)₂, 13 R⁶S(O)ₜN(R⁷)N(R⁷)₂, —R⁶S(O)ₜN(R⁷)N═C(R⁷)₂, —R⁶S(O)ₜN(R)⁷)C(O)R⁸, —R⁶S(O)ₜN(R⁷)C(O)N(R⁷)₂, —R⁶S(O)ₜN(R⁷)C(NR⁷)₂, —R⁶N(R⁷)C(O)R⁸, —R⁶N(R⁷)C(O)OR⁸, —R⁶N(R⁷)C(O)N(R⁷)₂, —R⁶N(R⁷)C(NR⁷)N(R⁷)₂, —RN(R⁷)C(S)N(R⁷)₂, and —R⁶N(R⁷)S(O)ₜR⁸, or
   (ii) any two adjacent R¹ groups together form an alkylenedioxy group; each R⁶ is independently a direct bond, alkylene chain or alkenylene chain; each R⁷ is independently selected from (i) or (ii) below:
      (i) each R⁷ is selected from a group consisting of hydrogen, alkyl, alkenyl, alkynyl, cycloalkyl, cycloalkylalkyl, aryl, aralkyl, heterocyclyl, heterocyclylalkyl, heteroaryl and heteroaralkyl, or
      (ii) two R⁷ groups together with the N atom to which they are attached form a heterocyclyl or heteroaryl;
each R⁸ is independently selected from a group consisting of alkyl, alkenyl, alkynyl, cycloalkyl, cycloalkylalkyl, aryl, aralkyl, heterocyclyl, heterocyclylalkyl, heteroaryl and heteroaralkyl;
each R⁹ is independently an alkylene chain or an alkenylene chain;
R² is hydrogen, halo, alkyl, amino or alkylamino;
R³ is halo or alkyl;
R⁴ and R⁵ are selected as follows:
   a) R⁴ and R⁵ are each independently hydrogen or alkyl, or
   b) R⁴ and R⁵, together with the N atom to which they are attached, form as oxo-substituted heterocyclyl;
R¹¹ is aryl, heteroaryl or heterocyclyl;
m is an integer from 0 to 4;
n is an integer from 0 to 4;
t is an integer from 0 to 2;
R¹, R², R³, R⁴, R⁵, R⁶, R⁷, R⁸, R⁹ and R¹¹ are optionally substituted with one, two or three substituents independently selected from Q¹, wherein Q¹ is nitro, halo, azido, cyano, oxo, thioxo, imino, alkyl, alkenyl, alkynyl, cycloalkyl, cycloalkylalkyl, aryl, aralkyl, heteroaryl, heteroaralkyl heterocyclyl, heterocyclylalkyl, —R^{u}OR^{x}, —R^{u}OR^{u}OR^{x}, —R^{u}OR^{u}N(R^{y})(R^{z}), —R^{u}N(R^{y})(R^{z}),
   —R^{u}SR^{x}, —R^{u}C(J)R^{x}, —R^{u}C(J)OR^{x}, —R^{u}C(J)N(R^{y})(R^{z}), —R^{u}C(J)SR^{x}, —R^{u}S(O)^{t}R^{w}, —R^{u}OC(J)R^{x}, —R^{u}OC(J)OR^{x}, —^{u}OC(J)N(R^{y})(R^{z}), —R^{u}OC(J)SR^{x}, —R^{u}N(R^{x})C(J)R^{x}, —R^{u}N(R^{x})C(J)OR^{x}, —R^{u}N(R^{x})C(J)N(R^{y})(R^{z}), —R^{u}N(R^{x})C(J)SR^{x}, —R^{u}Si(R^{w})₃, —R^{u}N(R^{x})S(O)ₜR^{w}, —R^{u}N(R^{x})R^{u}S(O)₂,R^{w}, —R^{u}N(R^{x})S(O)₂N(R^{y})(R^{z}), —R^{u}S(O)₂N(R^{y})(R^{z}), —R^{u}P(O)(R^{v})₂,—R^{u}OP(O)R^{v})₂, —R^{u}C(J)N(R^{x})S(O)₂R^{w}, —R^{u}C(J)N(R^{x})N(R^{x})S(O)₂R^{w}, —R^{u}C(R^{x})═(OR^{x}) and —R^{u}C(R^{x})═NN(R^{y})(R^{z});
when Q¹ is alkyl, alkenyl or alkynyl, each Q¹ is optionally substituted with halo, cyano, hydroxy or alkoxy;
when Q¹ is cycloalkyl, cycloalkylalkyl, aryl, aralkyl, heteroaryl, heteroaralkyl, heterocyclyl, or heterocyclylalkyl, each Q¹ is optionally substituted with halo, alkyl, haloalkyl, hydroxyalkyl, alkoxyalkyl, cyanoalkyl, alkoxy or hydroxyl;
each R^{u} is independently alkylene or a direct bond;
each R^{v} is independently alkyl, alkenyl, alkynyl, cycloalkyl, cycloalkylalkyl, heterocyclyl, heterocyclylalkyl, aryl, aralkyl heteroaryl, heteroaralkyl, hydroxy,—OR^{x} or —N(R^{y})(R^{z});
R^{w} is alkyl, alkenyl, alkynyl, cycloalkyl, cycloalkylalkyl, heterocyclyl, heterocyclylalkyl, aryl, aralkyl, heteroaryl, or heteroaralkyl;
each R^{x} is independently hydrogen, alkyl, alkenyl, alkynyl, cycloalkyl, cycloalkylalkyl, heterocyclyl, heterocyclylalkyl, aryl, aralkyl, heteroaryl, or heteroaralkyl;
R^{y} and R^{z} are each independently hydrogen, alkyl, alkenyl, alkynyl, cycloalkyl, cycloalkylalkyl, heterocyclyl, heterocyclylalkyl, aryl, aralkyl, heteroaryl, or heteroaralkyl;
R^{y} and R^{z} together with the nitrogen atom to which they are attached, form a heterocyclyl or heteroaryl; and
J is O, NR^{x} or S.

In some embodiments, the compound has a structure of formula C-II:

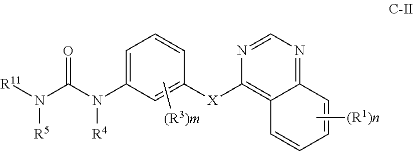

or a pharmaceutically acceptable salt or prodrug thereof, wherein
X is O, S, S(O) or SO₂;
each R¹ is independently selected from (i) or (ii) below;
   (i) each R¹ is independently selected from the group consisting of halo, alkyl, alkenyl, alkynyl, cycloalkyl, cycloalkylalkyl, heterocyclyl, heterocyclylalkyl, aryl, aralkyl, heteroaryl, heteroaralkyl, —R⁶OR⁷, —R⁶SR⁷, —R⁶S(O)ₜR⁸, —R⁶N(R⁷)₂, —R⁶OR⁹OR⁷, —R⁶OR⁹S(O)ₜR⁸, —R⁶OR⁹S(O)ₜN(R⁷)₂, —R⁶OR⁹N(R7)₂, —R⁶SR⁹OR⁷, —R⁶SR⁹SR⁷, —R⁶SR⁹N(R⁷)₂, —R⁶N(R⁷)R⁹N(R⁷)₂, —R⁶N(R⁷)R⁹OR⁷, —R⁶N(R⁷)R⁹SR⁷, —R⁶CN, —R⁶C(O)R⁷, —R⁶C(O)OR⁹OR⁷, —R⁶C(O)N(R⁷)₂, —R⁶C(O)N(R⁷)OR⁷, —R⁶C(O)N(R⁷)R⁹OR⁷, —R⁶C(O)N(R⁷)R⁹SR⁷, —R⁶C()SR⁸, —R⁶S(O)ₜOR⁷, —R⁶S(O)ₜN(R⁷)₂; or
   (ii) any two adjacent R¹ groups together form an alkylenedioxy group;
each R⁶ is independently a direct bond, alkylene chain or alkenylene chain;
each R⁷ is independently selected from (i) or (ii) below:
   (i) each R⁷ is selected from the group consisting of hydrogen, alkyl, alkenyl, alkynyl, cycloalkyl, cycloalkylalkyl, aryl, aralkyl, heterocyclyl, heterocyclylalkyl, heteroaryl and heteroaralkyl, or
   (ii) two R⁷ groups together with the N atom to which they are attached form a heterocyclyl or heteroaryl;
each R⁹ is independently an alkylene chain or an alkenylene chain;
R³ is halo or alkyl;
R⁴ and R⁵ are each independently hydrogen or alkyl;
R¹¹ is aryl or heteroaryl;
m is an integer from 0 to 4;
n is an integer from 0 to 4;
R, R², R³, R⁴, R⁵, R⁶, R⁷, R⁸, R⁹ and R¹¹ are optionally substituted with one, two or three substituents independently selected from Q¹, wherein Q¹ is nitro, halo, azido, cyano, oxo, thioxo, imino, alkyl, alkenyl, alkynyl, cycloalkyl, cycloalkylalkyl, aryl, aralkyl, heterocyclyl, heterocyclylalkyl, heteroaryl, heteroaralkyl, —R^{u}OR^{x}, —R^{u}OR^{u}OR^{x}, —R^{u}OR^{u}N(R^{y})(R^{z}), —R^{u}N(R^{y})(R^{z}), —R^{u}SR^{x}, —R^{u}C(J)R^{x}, —R^{u}C(J)OR^{x}, —R^{u}C(J)N(R^{y})(R^{z}), —R^{u}C(J)SR^{x}, —R^{u}S(O)ₜR^{w}, —R^{u}OC(J)R^{x}, —R^{u}OC(J)OR^{x}, —R^{u}OC(J)N(R^{y})(R^{z}), —R^{u}OC(J)SR^{x}, —R^{u}N(R^{x})C(J)R^{x}, —R^{u}N(R^{x})C(J)OR^{x}, —R^{u}N(R^{x})C(J)N(R^{y}(R^{z}), —R^{u}N(R^{x})C(J)SR^{x}, —R^{u}Si(R^{w})₃, —R^{u}N(R^{x})S(O)₂R^{w}, —R^{u}N(R^{x}) R^{u}S(O)₂R^{w}, —R^{u}N(R^{x})S(O)₂N(R^{y})(R^{z}), —R^{u}S(O)₂N(R^{y})(R^{z}), —R^{u}P(O)(R^{v})₂, —R^{u}OP(O)(R^{v})₂, —R^{v}C(J)N(R^{x})S(O)₂R^{w}, —R^{u}C(J)N(R^{x})N(R^{x})S(O)₂R^{w}, —R^{u}C(R^{u})═N(OR^{x}) and —R^{u}C(R^{x})═NN(R^{y})(R^{z});
when Q¹ is alkyl, alkenyl or alkynyl, each Q¹ is optionally substituted with halo, cyano, hydroxy or alkoxy;
when Q¹ is cycloalkyl, cycloalkylalkyl, aryl, aralkyl, heteroaryl, heteroaralkyl, heterocyclyl, or heterocyclylalkyl, each Q¹ is optionally substituted with halo, alkyl, haloalkyl, hydroxyalkyl, alkoxyalkyl, cyanoalkyl, alkoxy, hydroxyl, oxo or cyano;
each R^{u} is independently alkylene or a direct bond;
each R^{v} is independently alkyl, alkenyl, alkynyl, cycloalkyl, cycloalkylalkyl, heterocyclyl, heterocyclylalkyl, aryl, aralkyl, heteroaryl, heteroaralkyl, hydroxy, —OR^{x} or —N(R^{y})(R^{z});
R^{w} is alkyl, alkenyl, alkynyl, cycloalkyl, cycloalkylalkyl, heterocyclyl, heterocyclylalkyl, aryl, aralkyl, heteroaryl, or heteroaralkyl;
each R^{x} is independently hydrogen, alkyl, alkenyl, alkynyl, cycloalkyl, cycloalkylalkyl, heterocyclyl, heterocyclylalkyl, aryl, aralkyl, heteroaryl, or heteroaralkyl;
R^{y} and R^{z} are each independently hydrogen, alkyl, alkenyl, alkynyl, cycloalkyl, cycloalkylalkyl, heterocyclyl, heterocyclylalkyl, aryl, aralkyl, heteroaryl, or heteroaralkyl;
R^{y} and R^{z}, together with the nitrogen atom to which they are attached, form a heterocycle or heteroaryl;
t is an integer from 0 to 2; and
J is O, NR^{x} or S.

In some of the above embodiments, the compound has a structure of formula C-III:

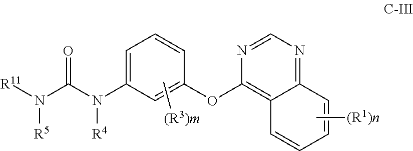

or a pharmaceutically acceptable salt or prodrug thereof,

In some of the above embodiments, the compound has a structure of formula C-XII:

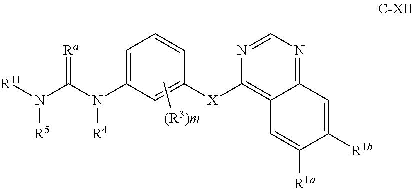

or a pharmaceutically acceptable salt or prodrug thereof, wherein R^{a} is O or S;
X is O or S;
R^{1a} and R^{1b} are selected as follows:
   i) R^{1a} and R^{1b} are each independently selected from hydrogen, halo, amino, alkyl, aryl, heteroaryl, alkoxy, hydroxy, alkoxyalkoxy, cycloalkylcarbonylamino and a group of formula:

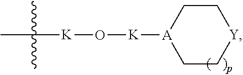

where each K is independently a direct bond or alkylone;
A is N or CR¹⁶;
Y is —O, —S, —S(O), —S(O)₂—N(R¹⁴), —C(H)R¹⁵, or —C(O);
p is an integer from 0 to 2;
R¹⁴ is hydrogen, alkyl, haloalkyl, hydroxyalkyl, cycloalkyl, heteroarylalkyl, arylalkyl, S(O)₁R¹³ or —C(O)R¹²;
R¹⁵ is hydrogen, halo, alkyl, hydroxyalkyl or —OR¹²;
R¹⁶ is hydrogen or alkyl;
t is 1 or 2;
each R¹² is independently selected from the group consisting of hydrogen and alkyl;
R¹³ is alkyl;
each K is optionally substituted with one, two or three hydroxy or alkyl groups; or
   ii) R^{1a} and R^{1b} groups together form an alkylenedioxy group.

In some of the above embodiments, the compound has a structure of formula C-XVII:

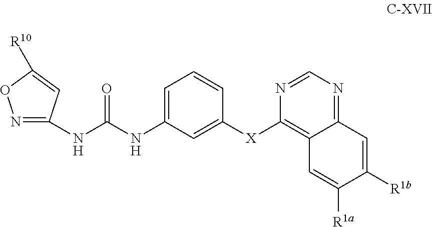

or a pharmaceutically acceptable salt or prodrug thereof,
wherein X is O or S;
R^{1a} and R^{1b} are selected as follows:
   i) R^{1a} and R^{1b} are each independently alkoxy, alkoxyalkoxy, alkylsulfonylalkoxy or a group of formula:

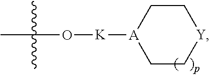

where K is a direct bond or alkylene, optionally substituted with a hydroxy group;
A is M or CH;
Y is —O, —S(O)₂, —N(R14) or —C(H)R¹⁵; p1 is 0 or 1;
R¹⁴ is hydrogen, alkyl, haloalkyl, hydroxyalkyl or S(O)₁R¹³;
R¹⁵ is hydrogen, halo, alkyl, hydroxyalkyl or —OR¹²;
t is 1 or 2;
R¹² is hydrogen or alkyl; and
R¹³ is alkyl; or
   ii) R^{1a} and R^{1b} groups together form an alkylenedioxy group; and
R¹⁰ is hydrogen, halo, alkyl, aryl, heterocyclyl, heteroaryl, cycloalkyl or cycloalkylalkyl; where alkyl, aryl, heterocyclyl and heteroaryl groups are optionally substituted with 1-3 groups selected from halo, cyano, hydroxyl and alkoxy.

In some embodiments, the compound is selected from:
1-(5-tert-butylisoxazol-3-yl)-3-(3-(6,7-dimethoxyquinazolin-4-yloxy)phenyl)urea;
1-(5-tert-butylisoxazol-3-yl)-3-(3-(6-methoxyquinazolin-4-yloxyphenyl)urea;
1-(5-tert-butylisoxazol-3-yl)-3-(3-(7-methoxyquinazolin-4-yloxy)phenyl)urea;
1-(5-tert-butylisoxazol-3-yl)-3-(6,7-difluoroquinazolin-4-yloxy)phenyl)urea;
1-(5-tert-butylisoxazol-3-yl)-3-(3-(5-methylquinazolin-4-yloxy)phenyl)urea;
1-(5-tert-butylisoxazol-3-yl)-3-[3-(7-ethoxy-6-methoxyquinazolin-4-yloxy)phenyl] urea hydrochloride;
1-(5-tert-Butylisoxazol-3-yl)-3-{3-[6-methoxy-7-(2-methoxyethoxy)quinazolin-4-yloxy]phenyl}urea hydrochloride;
1-(5-tert-butylisoxazol-3-yl)-3-(3-(6-methylquinazolin-4-yloxy)phenyl)urea;
1-(5-tert-butylisoxazol-3-yl)-3-(3-(6,7-dimethoxyquinazolin-4-yloxy)-4-fluorphenyl)urea;
1-(5-tert-butylisoxazol-3-yl)-3-(4-chloro-3-(6,7-dimethoxyquinazolin-4-yloxy)phenyl)urea;
1-(5-tert-butylisoxazol-3-yl)-3-(3-(6-ethoxy-7-methoxyquinazolin-4-yloxy)phenyl)urea;
1-(3-[6,7-bis(2-methoxyethoxy)quinazolin-4-yloxy]phenyl}-3-(5-tert-butylisoxazol-3-yl)urea hydrochloride;
1-(5-tert-Butylisoxazol-3-yl)-3-[3-(6,7-diethyoxyquinazolin-4-yloxy)phenyl]urea hydrochloride;
1-(5-tert-Butylisoxazol-3-yl)-3-[3-(7,8-dihydro-[1,4]dioxino[2,3-g]quinazolin-4-yloxy)phenyl]urea hydrochloride;
1-(5-tert-butylisoxazol-3-yl)-3-{3-[7-methoxy-6-(2-methoxyethoxy)quinazolin-4-yloxy]phenyl}urea hydrochloride;
1-(5-tert-butylisoxazol-3-yl)-3-(3-(7-methoxy-6-(2-piperidin-1-yl)ethoxy)quinazolin-4-yloxy)phenyl)urea;
1-(5-tert-butylisoxazol-3-yl)-3-(3-(6-(2-(4-(hydroxymethyl)piperidin-1-yl)ethoxy)-7-methoxyquinazolin-4-yloxy)phenyl)urea;
1-(5-tert-butylisoxazol-3-yl)-3-(3-(7-methoxy-6-(2-(4-methylpiperazin-1-yl)ethoxy)quinazolin-4-yloxy)phenyl)urea;
1-(5-tert-butylisoxazol-3-yl)-3-(3-(6-(2-(4-(2-hydroxyethyl)piperazin-1-yl)ethoxy)-7-methoxyquinazolin-4-yloxy)phenyl)urea;
1-(5-tert-butylisoxazol-3-yl)-3-(3-(7-methoxy-6-(2-morpholinoethoxy)quinazolin-4-yloxy)phenyl)urea;
1-(5-tert-butylisoxazol-3-yl)-3-(3-(7-methoxy-6-(3-(4-methylpiperazin-1-yl)propoxy)quinazolin-4-yloxy)phenyl)urea;
1-(5-tert-butylisoxazol-3-yl)-3-(3-(7-methoxy-6-(3-morpholinopropoxy)quinaxzolin-4-yloxy)phenyl)urea;
1-(5-tert-butylisoxazol-3-yl)-3-(3-(7-methoxy-6-(3-(piperidin-1-yl)propoxy)quinazolin-4-yloxy)phenyl)urea;
1-(5-tert-butylisoxazol-3-yl)-3-(3-(6-(3-(4-(hydroxymethyl)piperidin-1-yl)propoxy)-7-methoxyquinazolin-4-yloxy)phenyl)urea;
1-(5-tert-butylisoxazol-3-yl)3-(3-(7-methoxy-6-(3-(4-(methylsulfonyl)piperaxin-1-yl)propoxy)quinazolin-4-yloxy)phenyl)urea;
1-(5-tert-butyl-isoxazol-3-yl)-3-(3-{6-[3-(1,1-dioxo-thiomorpholin-4-yl)propoxy]-7-methoxy-quinazolin-4-yloxy}-phenyl)-urea;
1-(5-tert-butylisoxazol-3-yl)-3-(3-(6-methoxy-7-(3-morpholinopropoxy)quinazolin-4-yloxy)phenyl)urea;
1-(5-tert-butylisoxazol-3-yl)-3-(3-(6-methoxy-7-(3-(4-methylpiperazin-1-yl)propoxy)quinazolin-4-yloxy)phenyl)urea;
1-(5-tert-butylisoxazol-3-yl)-3-(3-(7-(3-(4-hydroxymethyl)piperidin-1-yl)propoxy)-6-methoxyquinazolin-4-yloxy)phenyl)urea;
1-(5-tert-butylisoxazol-3-yl)-3-(3-(7-(3-(4-(2-hydroxyethyl)piperazin-1-yl)propoxy)-6-methoxyquinazolin-4-yloxy)phenyl)urea;
1-(5-tert-butyl-isoxazol-3-yl)-3-(3-{7-[3-(3-hydroxy-pyrrolidin-1-yl)-propoxy]-6-methoxy-quinazolin-4-yloxy}-phenyl)-urea;
1-(5-tert-butylisoxazol-3-yl)-3-(3-(6-methoxy-7-(3-(4-(methylsulfonyl)piperazin-1-yl)propoxy)quinazolin-4-yloxy)phenyl)urea;
(S)-1-(5-tert-butylisoxazol-3-yl)-3-(3-(7-(3-(3-hydroxypyrrolidin-1-yl)propoxy)-6-methoxyquinazolin-4-yloxy)phenyl)urea;
(R)-1-(5-tert-butylisoxazol-3-yl)-3-(3-(7-(3-(3-hydroxypyrrolidin-1-yl)propoxy)-6-methoxyquinazolin-4-yloxy)phenyl)urea;
1-(5-tert-butylisoxazol-3-yl)-3-(3-(6-methoxy-7-(2-morpholinoethoxy)quinazolin-4-yloxy)phenyl)urea;
1-(5-tert-butylisoxazol-3-yl)-3-(3-(6-methoxy-7-(2-(4-methyl piperazin-1-yl)ethoxy)quinazolin-4-yloxy)phenyl)urea;
1-(5-tert-Butyl-isoxazol-3-yl)-3-(3-{7-[2-(4-hydroxymethyl-piperidin-1-yl)-ethoxy]-6-methoxy-quinazolin-4-yloxy}-phenyl)-urea;
1-(5-tert-butylisoxazol-3-yl)-3-(3-(7-(2-(4-(2-hydroxymethyl-piperidin-1-yl)-ethoxy)-6 methoxyquinazolin-4yloxy)phenyl)urea;
1-(5-tert-Butyl-isoxazol-3-yl)-3-(3-{7-[2-(1,1-dioxo-116-thiomorpholin-4-yl)-ethoxy]-6-methoxy-quinazolin-4-yloxy}- phenyl)-urea;
1-(5-tert-butylisoxazol-3-yl)-3-(3-(6-(2-methoxyethoxy)quinazolin-4-yloxy)phenyl)urea;
1-(5-tert-Butylisoxazol-3-yl)-3-(3-(7-methoxy-6-(3-(methylsulfonyl)propoxy)quinazolin-4-ylthio)phenyl)urea;
1-(3-(2-fluoropropan-2-yl)isoxazol-5-yl)-3-(3-(7-methoxy-6-(3-(methylsulfonyl)propoxy)quinazolin-4-yloxy)phenyl)urea;
1-(5-tert-butylisoxazol-3-yl)-3-(3-(7-(2-methoxyethoxy)quinazolin-4-yloxy)phoayl) urea;
1-(5-tert-butylisoxazol-3-yl)-3-(3-(7-methoxy-6-(3-(methylsulfonyl)propoxy)quinazolin-4-ylthio)phenyl)urea;
1-(5-tert-butylisoxazol-3-yl)-3-(3-(7-methoxy-6-(2-methoxyethoxy)quinazolin-4-ylthio)phenyl)urea;
1-(5-tert-butylisoxazol-3-yl)-3-(3-(6,7-dimethoxyquinazolin-4-ylthio)phenyl)urea;
1-(5-tert-butylisoxazol-3-yl)-3-(3-(6,7-difluoroquinazolin-4-ylthio)phenyl)urea;
1-(5-tert-butylisoxazol-3-yl)-3-(3-(7-methoxyquinazolin-4-ylthio)phenyl)urea;
1-(5-tert-butylisoxazol-3-yl)-3-(3-(6-methoxyquinazolin-4-ylthio)phenyl)urea;
1-(5-tert-Butylisoxazol-3-yl)-3-[3-(7-ethoxy-6-methoxyquinazolin-4-ylthio)phenyl]urea;
1-(5-tert-butylisoxazol-3-yl)-3-[3-(6,7-diethoxyquinazolin-4-ylthio)phenyl]urea;
1-(5-tert-butylisoxazol-3-yl)-3-{3-[6-methoxy-7-(2-methoxyethoxy)quinazolin-4-ylthio]phenyl}urea hydrochloride;
1-{3-[6,7-bis(2-methoxyethoxy)quinazolin-4-ylthio]phenyl}-3-(5-tert-butylisoxazol-3-yl)urea hydrochloride;
1-(5-tert-butylisoxazol-3-yl)-3-[3-(7,8-dihydro-[1,4]dioxino[2,3-g]quinazolin-4-ylthio)phenyl]urea hydrochloride;
1-(5-tert-Butylisoxazol-3-yl)-3-{3-[7-methoxy-5-(tetrahydro-2H-pyran-4-ylthio)quinazolin-4-yloxy]phenyl}urea;
1-(5-tert-butylisoxazol-3-yl)-3-(3-(6-ethoxy-7-methoxyquinazolin-4-ylthio)phenyl)urea;
1-(5-tert-butylisoxazol-3-yl)-3-(3-(7-methoxy-6-(3-(piperidin-1-yl)propoxy)quinazolin-4-ylthio)phenyl)urea;
1-(5-tert-butylisoxazol-3-yl)-3-(3-(6-(3-(4-(hydroxymethyl)piperidin-1-yl)propoxy)-7-methoxyquinazolin-4-ylthio)phenyl)urea;
1-(5-tert-Butylisoxazol-3-yl)-3-(3-(7-methoxy-6-(3-(4-methylpiperazin-1-yl)propoxy)quinazolin-4-ylthio)phenyl)urea;
1-(5-tert-butylisoxazol-3-yl)-3-(3-(7-methoxy-6-(3-(4-(methylsulfonyl)piperazin-1-yl)propoxy)quinazolin-4-ylthio)phenyl)urea;
1-(5-tert-butylisoxazol-3-yl)-3-(3-(6-(3-(4-(2-hydroxyethyl)piperazin-1-yl)propoxy)-7-methoxyquinazolin-4-ylthio)phenyl)urea;
1-(5-tert-butyl-isoxazol-3-yl)-3-(3-{6-[3-(1,1-dioxo-thiomorpholin-4-yl)-propoxy]-7-methoxy-quinazolin-4-ylsulfanyl}-phenyl)- urea;
1-(5-tert-butylisoxazol-3-yl)-3-(3-(7-methoxy-6-(3-morpholinopropoxy)quinazolin-4-ylthio)phenyl)urea;
1-(5-tert-butylisoxazol-3-yl)-3-(3-(7-methoxy-6-(3-(methylsulfonyl)propoxy)quinazolin-4-ylthio)phenyl)urea;
1-(5-tert-butylisoxazol-3-yl)-3-(3-(7-methoxy-6-(2-(piperidin-1-yl)ethoxy)quinazolin-4-ylthio)phenyl)urea;
1-(5-tert-butylisoxazol-3-yl)-3-(3-(6-(2-(4-(hydroxymethyl)piperidin-1-yl)ethoxy)-7-methoxyquinazolin-4-ylthio)phenyl)urea;
1-(5-tert-butylisoxazol-3-yl)-3-(3-(7-methoxy-6-(2-(4-methylpiperazin-1-yl)ethoxy)quinazolin-4-ylthio)phenyl)urea;
1-(5-tert-butylisoxazol-3-yl)-3-(3-(6-(2-(4-(2-hydroxyethyl)piperazin-1-yl)ethoxy)-7-methoxyquinazolin-4-ylthio)phenyl)urea;
1-(5-tert-butylisoxazol-3-yl)-3-(3-(7-methoxy-6-(2-(4-(methylsulfonyl)piperazin-1-yl)ethoxy)quinazolin-4-ylthio)phenyl)urea;
1-(5-tert-butylisoxazol-3-yl)-3-(3-(7-methoxy-6-(2-morpholinoethoxy)quinazolin-4-ylthio)phenyl)urea;
1-(5-tert-butyl-isoxazol-3-yl)-3-(3-{6-[2-(1,1-dioxo-thiomorpholin-4-yl)-ethoxy]-7-methoxy-quinazolin-4-ylsulfanyl}-phenyl)- urea;
1-(5-tert-butylisoxazol-3-yl)-3-(3-(6-methoxy-7-(2-morpholinoethoxy)quinazolin-4-ylthio) phenyl)urea;
1-(5-tert-butylisoxazol-3-yl)-3-(3-(6-methoxy-7-(3-(4-methylpiperazin-1-yl)propoxy)quinazolin-4-ylthio)phenyl)urea;
1-(5-tert-butylisoxazol-3-yl)-3-(3-(7-(3-(4-(hydroxymethyl)piperidin-1-yl)propoxy)-6-methoxyquinazolin-4-ylthio)phenyl)urea;
1-(5-tert-butylisoxazol-3-yl)-3-(3-(7-(3-(4-(2-hydroxyethyl)piperazin-1-yl)propoxy)-6-methoxyquinazolin-4-ylthio)phenyl)urea;
1-(5-tert-butylisoxazol-3-yl)-3-(3-(6-methoxy-7-(3-(piperidin-1-yl)propoxy)quinazolin-4-ylthio)quinazolin-4-yl thio)phenyl)urea;
1-(5-tert-butylisoxazol-3-yl)-3-(3-(6-methoxy-7-(3-(4-(methylsulfonyl)piperazin-1-yl)propoxy)quinazolin-4-ylthio)phenyl)urea;
1-(5-tert-butylisoxazol-3-yl)-3-(3-(6-methoxy-7-(2-morpholinoethoxy)quinazolin-4-ylthio) phenyl)urea;
1-(5-tert-butylisoxazol-3-yl)-3-(3-(6-methoxy-7-(2-(piperidin-1-yl)ethoxy)quinazolin-4-ylthio)phenyl)urea;
1-(5-tert-butylisoxazol-3-yl)-3-(3-(6-methoxy-7-(2-(4-(methylsulfonyl)piperazin-1-yl)ethoxy)quinazolin-4-ylthio)phenyl)urea;
1-(5-tert-butylisoxazol-3-yl)-3-(3-(6-methoxy-7-(2-(4-methylpiperazin-1-yl)ethoxy)quinazolin-4-ylthio)phenyl)urea;
1-(5-tert-butylisoxazol-3-yl)-3-(3-(7-(2-(4-(2-hydroxyethyl)piperazin-1-yl)ethoxy)-6-methoxyquinazolin-4-ylthio)phenyl)urea;
1-(5-tert-butylisoxazol-3-yl)-3-(3-(7-(2-(4-(hydroxymethyl)piperidin-1-yl)ethoxy)-6-methoxyquinazolin-4-ylthio)phenyl)urea;
1-(5-tert-butylisoxazol-3-yl)-3-(3-(6-(2-methoxyethoxy)quinazolin-4-ylthio)phenyl)urea;
1-(5-tert-butylisoxazol-3-yl)-3-(3-(7-methoxy-6-(2-methylsulfonyl)ethoxy)quinazolin-4-ylthio)phenyl)urea;
1-(5-tert-butylisoxazol-3-yl)-3-(3-(2-chloro-6,7-dimethoxyquinazolin-4-ylthio) phenyl)urea;
1-(5-tert-butylisoxazol-3-yl)-3-(3-{6-[3-(1,1-dioxo-thiomorpholin-4-yl)-propoxy]-quinazolin-4-ylsulfanyl}-phenyl)-urea;
1-(5-tert-Butyl-isoxazol-3-yl)-3-(3-{6-[2-(1,1-dioxo-116-thiomorpholin-4-yl)-ethoxy]-7-methoxy-quinazolin-4-yloxy}- phenyl)-urea;
1-(5-tert-butylisoxazol-3-yl)-3-{3-(6-(5-{[2-(methylsulfonyl)ethylamino]methyl}furan-2-yl)quinazolin-4-yloxy]phenyl}urea;
1-(5-tert-butylisoxazol-3-yl)-3-{3-[7-methoxy-5-(tetrahydro-2H-pyran-4-yloxy)quinazolin-4-yloxy]phenyl}urea;
1-(5-tert-butylisoxazol-3-yl)-3-(3-(7-hydroxy-6-methoxyquinazolin-4-yloxy)phenyl)urea;
1-(5-tert-butylisoxazol-3-yl)-3-(3-(6-methoxy-7-(pyrrolidin-3-yloxy) quinazolin-4-yloxy)phenyl)urea;
(S)-1-(5-tert-butylisoxazol-3-yl)-3-(3-(6-methoxy-7-(1-methylpyrrolidin-3-yloxy)quinazolin-4-yloxy)phenyl);urea mono acetate;
(R)-1-(5-tert-butylisoxazol-3-yl)-3-(3-(6-methoxy-7-(pyrrolidin-3-yloxy)quinazolin-4-yloxy)phenyl)urea;
(R)-1-(5-tert-butylisoxazol-3-yl)-3-(3-(6-methoxy-7-(1-methylpyrrolidin-3-yloxy)quinazolin-4-yloxy)phenyl)urea mono acetate;
(R)-1-(5-tert-butylisoxazol-3-yl)-3-(3-(7-(2-hydroxy-3-(4-methylpiperazin-1-yl)propoxy)-6-methoxyquinazolin-4-yloxy)phenyl)urea;
1-(3-tert-Butylisoxazol-5-yl)-3-(3-(6-methoxy-7-(piperidin-43-ylmethoxy)quinazolin-4-yloxy)phenyl)urea;
1-(3-tert-butylisoxazol-5-yl)-3-(3-(6-methoxy-7-((1-methylpiperidin-4-yl)methoxy)quinazolin-4-yloxy)phenyl)urea;
(S)-1-(5-tert-butylisoxazol-3-yl)-3-(3-{7-[1-(2,2-difluoroethyl)pyrrolidin-3-yloxy]-6-methoxyquinazolin-4-yloxy}phenyl)urea;
(S)-1-(5-tert-Butylisoxazol-3-yl)-3-(3-{6-methoxy-7-[1-(2,2,2-trifluoroethyl)pyrrolidin-3-yloxy]quinazolin-4-yloxy}phenyl)urea;
1-(5-tert-butylisoxazol-3-yl)-3-(3-{7-[1-(2,2-difluoroethyl)piperidin-4-yloxy]-6-methoxyquinazolin-4-yloxy}phenyl urea;
1-(5-tert-butylisoxazol-3-yl)-3-(3-(6-hydroxy-7-methoxyquinazolin-4-yloxy)phenyl)urea;
(S)-tert-butyl 3-(4-(3-(3-(5-tert-butylisoxazole-3-yl)ureido)phenoxy)-7-methoxyquinazolin-6-yloxy)pyrrolidine-1-carboxylate;
(S)-1-(5-tert-butylisoxazol-3-yl)-3-(3-(7-methoxy-6-(1-methylpyrrolidin-3-yloxy)quinazolin-4-yloxy)phenyl)urea;
(S)-1-(5-tert-butylisoxazol-3-yl)-3-(3-(6-(1-(2,2-difluoroethyl)pyrrolidin-3-yloxy)-7-methoxyquinazolin-4-yloxy)phenyl)urea;
(S)-1-(5-tert-butylisoxazol-3-yl)-3-(3-(6-(2-hydroxy-3-(4methylpiperazin-1-yl)propoxy)-7-methoxyquinazolin-4-yloxy)phenyl)urea;
(R)-3-(5-tert-butylisoxazol-3-yl)-3-(3-(6-(2-hydroxy-3-(4methylpiperazine-1-yl)propoxy)-7-methoxyquinazolin-4-yloxy)phenyl)urea;
1-(3-(6,7-dimethoxyquinazolin-4-yloxy)phenyl)-3-(5-phenylisoxazol-3-yl)urea;
1-(3-(6,7-dimethoxyquinazolin-4-ylthio)phenyl)-3-(3-methoxy-5-(trifluoromethyl)phenyl)urea;
1-(3-(6,7-dimethoxyquinazolin-4-yloxy)phenyl)-3-(3-methoxy-5-(trifluoromethyl)phenyl)urea;
1-(3-(6-methoxy-7-(2-methoxyethoxy)quinazolin-4-yloxy)phenyl)-3-(3-(2-methoxyethoxy)-5-(trifluoromethyl)phenyl)urea;
1-(3-tert-butylphenyl)-3-(3-(6,7-dimethoxyquinazolin-4-yloxy)phenyl)urea;
1-(3-tert-butylphenyl)-3-(3-(6-methoxy-7-(2-methoxyethoxy)quinazolin-4-yloxy)phenyl)urea;
1-(3-tert-butylphenyl)-3-(3-(6,7-dimethoxyquinazolin-4-ylthio)phenyl)urea;
1-(3-(6,7-dimethoxyquinazolin-4-yloxy)phenyl)-3-(3-isopropylisoxazol-5-yl)urea;
1-(3-(6,7-dimethoxyquinazolin-4-yloxy)phenyl)-3-(3-(tetrahydro-2H-pyran-4-yl)isoxazol-5-yl)urea;
1-(3-cyclopropylisoxazol-5-yl)-3-(3-(6,7-dimethoxyquinazolin-4-yloxy)phenyl)urea;
1-(3-(2-cyanopropan-2-yl)isoxazol-5-yl)-3-(3-(6,7-dimethoxyquinazolin-4-yloxy)phenyl)urea;
1-(3-(6,7-dimethoxyquinazolin-4-yloxy)phenyl)-3-(3-(2-fluoropropan-2-yl)isoxazol-5-yl)urea;
1-(3-(6,7-dimethoxyquinazolin-4-yloxy)phenyl)-3-(5-(1-methylcyclopropyl)isoxazol-3-yl)urea;
1-(3-(6,7-dimethoxyquinazolin-4-yloxy)phenyl)-3-(3-(2-methoxyethoxy)-5-(trifluoromethyl)phenyl)urea;
1-(3-(6,7-dimethoxyquinazolin-4-yloxy)phenyl)-3-(5-(1-hdroxy-2-methylpropan-2-yl)isoxazol-3-yl)urea;
1-(3-tert-butylisoxazol-5-yl)-3-(3-(6,7-dimethoxyquinazolin-4-yloxy)phenyl)urea;
1-(3-(6,7-dimethoxyquinazolin-4-yloxy)phenyl)-3-(5-isopropylisoxazol-3-yl)urea;
1-(3-(6,7-dimethoxyquinazolin-4-ylthio)phenyl)-3-(5-isopropylisoxazol-3-yl)urea;
1-(5-cyclopentylisoxazol-3-yl)-3-(3-(6,7-dimethoxyquinazolin-4-yloxy)phenyl)urea;
1-(3-(6,7-dimethoxyquinazolin-4-yloxy)phenyl)-3-(5-(2-fluoropropan-2-yl)isoxazol-3-yl)urea;
1-(3-(6,7-dimethoxyquinazolin-4-yloxy)phenyl)-3-(1-phenyl-3-(1-(trifluoromethyl)cyclopropyl)-1H-pyrazol-5-yl)urea;
1-(3-(6,7-dimethoxyquinazolin-4-yloxy)phenyl)-3-(4-methoxy-3-(trifluoromethyl)phenyl)urea;
1-(4-methoxy-3-(trifluorormethyl)phenyl)-3-(3-(6-methoxy-7-(2-methoxyethoxy)quinazolin-4-yloxy)phenyl)urea;
1-(3-chloro-5-(trifluoromethyl)phenyl)-3-(3-(6,7-dimethoxyquinazolin-4-yloxy)phenyl)urea;
1-(3-(6,7-dimethoxyquinazolin-4-yloxy)phenyl)-3-(4-trifluoromethyl)pyridin-2-yl)urea;
1-(2-chloro-5-(trifluoromethyl)phenyl)-3-(3-(6,7-dimethoxyquinazolin-4-yloxy)phenyl)urea;
1-(3-(6,7-dimethoxyquinazolin-4-yloxy)phenyl)-3-(4-(trifluoromethyl)pyrimidin-2-yl)urea;
1-(3-(6,7-dimethoxyquinazolin-4-yloxy)phenyl)-3-(3-isopropylphenyl)urea;
1-(3-(6,7-dimethoxyquinazolin-4-yloxy)phenyl)-3-(6-(trifluoromethyl)pyrimidin-4-yl)urea;
1-(3-(6,7-dimethoxyquinazolin-4-yloxy)phenyl)-3-(3-(2-methoxyethoxy)-4-(trifluoromethyl)phenyl)urea;
1-(3-(6,7-Dimethoxyquinazolin-4-ylthio)phenyl)-3-(3-(2-methoxyethyl)4-(trifluoromethyl)phenyl)urea;
1-(3-(6,7-dimethoxyquinazolin-4-yloxy)phenyl)-3-(3-(morpholine-4-carbonyl)-5-(trifluoromethyl)phenyl)urea;
1-(3-(6,7-dimethoxyquinazolin-4-yloxy)phenyl)-3-(3-fluoro-4-(trifluoromethyl)phenyl)urea;
1-(3-(6,7-dimethoxyquinazolin-4-yloxy)phenyl)-3-(3-(morpholinomethyl)-5-(trifluoromethyl)phenyl)urea;
1-(3-(1,1-difluoroethyl)isoxazol-5-yl)-3-(3-(6,7-dimethoxyquinazolin-4-yloxy)phenyl)urea;
1-(3-tert-butyl-1-phenyl-1H-pyrazol-5-yl)-3-(3-(6,7-dimethoxyquinazolin-4-yloxy)phenyl)urea;
1-(3-tert-butyl-1-phenyl-1H-pyrazol-5-yl)-3-(3-(6,7-dimethoxyquinazolin-4-ylthio)phenyl)urea;
1-(3-(6,7-dimethoxyquinazolin-4-yloxy)phenyl)-3-(3-(1-(trifluoromethyl)cyclobutyl)isoxazol-5-yl)urea;
1-(3-(6,7-dimethoxyquinazolin-4-ylthio)phenyl)-3-(3-(1-(trifluoromethyl)cyclobutyl)isoxazol-5-yl)urea;
1-(3-tert-butyl-1-methyl-1H-pyrazol-5-yl)-3-(3-(6,7-dimethoxyquinazolin-4-ylthio)phenyl)urea;
1-(3-tert-butyl-1-methyl-1H-pyrazol-5-yl)-3-(3-(6,7-dimethoxyquinazolin-4-yloxy)phenyl)urea;
1-[3-(1,3-difluoro-2-methylpropan-2-yl)isoxazol-5-yl]-3-[3-(6,7-dimethoxyquinazolin-4-yloxy]phenyl)urea;
1-[3-(1,3-difluoro-2-methylpropan-2-yl)isoxazol-5-yl]-3-[3-(6,7-dimethoxyquinazolin-4-ylthio]phenyl)urea;
1-[3-(6,7-dimethoxyquinazolin-4-yloxy)phenyl]-3-[1-phenyl-3-(trifluoromethyl)-1H-pyrazol-5-yl]urea;
1-[5-(1,3-difluoro-2-methylpropan-2-yl)isoxazol-3-yl]-3-[3-(6,7-dimethoxyquinazolin-4-yloxy)phenyl]urea;
1-3-cyclopentylisoxazol-5-yl)-3-(3-(6,7-dimethoxyquinazolin-4-yloxy)phenyl)urea;
1-[3-(6,7-dimethoxyquinazolin-4-yloxy)phenyl]-3-[1-methyl-3-(trifluoromethyl)-1H-pyrazol-5-yl]urea;
1-[3-(6,7-dimethoxyquinazolin-4-yloxy)phenyl]-3-[1-methyl-5-(trifluoromethyl)-1H-pyrazol-3-yl]urea;
ethyl 2-(3-tert-butyl-5-{3-[3-(6,7-dimethoxyquinazolin-4-yloxy)phenyl]ureido}-1H-pyrazol-1-yl)acetate;
1-[3-(1,3-difluoro-2-methylpropan-2-yl)-1-phenyl-1H-pyrazol-5-yl]-3-[3-(6,7-dimethoxyquinazolin-4-yloxy)phenyl]urea;
1-[3-(6,7-dimethoxyquinazolin-4-yloxy)phenyl]-3-[3-(2-ethoxypropan-2-yl)-1-phenyl-1H-pyrazol-5-yl]urea;
1-[3-(6,7-dimethoxyquinazolin-4-yloxy)phenyl]-3-[1-phenyl-5-(trifluoromethyl)-1H-pyrazol-3-yl]urea;
1-[3-(6,7-dimethoxyquinazolin-4-ylthio)phenyl]-3-[1-phenyl-5-(trifluromethyl)-1H-pyrazol-3-yl]urea;
1-[3-(6,7-dimethoxyquinazolin-4-yloxy)phenyl]-3-[1-(4-fluorophenyl)-3-(trifluoromethyl)-1H-pyrazol-5-yl]urea:
1-[3-(6,7-dimethoxyquinazolin-4-yloxy)phenyl]-3-[1-p-tolyl-3-(trifluoromethyl)-1H-pyrazol-5-yl]urea;
1-(4-tert-butylphenyl)-3-(3-(6,7-dimethoxyquinazolin-4-yloxy)phenyl)urea;
1-(4-tert-butylphenyl)-3-(3-(6,7-dimethoxyquinazolin-4-ylthio)phenyl)urea;
1-(4-chlorophenyl)-3-(3-(6,7-dimethoxyquinazolin-4-yloxy)phenyl)urea;
1-(4-chloro-3-(trifluoromethyl)phenyl)-3-(3-(6,7-dimethylquinazolin-4-yloxy)phenyl)urea;
1-(3-(6,7-dimethoxyquinazolin-4-yloxy)phenyl)-3-(4-(trifluoromethoxy)phenyl)urea;
1-(3-(6,7-dimethoxyquina:zolin-4-yloxy)phenyl)-3-(3-methoxyphenyl)urea;
1-(3-(6,7-dimethoxyquinazolin-4-yloxy)phenyl)-3-(3-ethoxyphenyl)urea;
1-(3-chloro-4-methoxyphenyl)-3-(3-(6,7-dimethoxyquinazolin-4-yloxy)phenyl)urea;
1-(3-(6,7-dimethoxyquinazolin-4-yloxy)phenyl)-3-(3-(trifluoromethyl)phenyl)urea;
1-(3-(6,7-dimethoxyquinazolin-4-yloxy)phenyl)-3-phenylurea;
1-(3-(6,7-dimethoxyquinazolin-4-yloxy)phenyl)-3-(4-trifluoromethyl)phenyl)urea;
1-(3-(6,7-dimethoxyquinazolin-4-yloxy)phenyl)-3-(4-(trifluoromethyl)phenyl)urea;
1-(3-(6,7-dimethoxyquinazolin-4-ylthio)phenyl)-3-(3-(trifluoromethyl)phenyl)urea;
1-(4-chloro-3-(trifluoromethyl)phenyl)-3-(3-(6,7-dimethoxyquinazolin-4-ylthio)phenyl)urea;
1-(3-(6,7-dimethoxyquinazolin-4-ylthio)phenyl)-3-(3-(2-fluoropropan-2-yl)isoxazol-5-yl)urea;
1-(3-(6,7-dimethoxyquinazolin-4-ylthio)phenyl)-3-(3-fluoro-4-(trifluoromethyl)phenyl)urea;
1-(3-(6,7-dimethoxyquinazolin-4-ylthio)phenyl)-3-(3-(morpholinomethyl)-5-(trifluoromethyl)phenyl)urea;
1-(3-(6,7-dimethoxyquinazolin-4-ylthio)phenyl)-3-(3-methoxy-4-(trifluoromethyl)phenyl)urea;
1-[5-(1,3-difluoro-2-methylpropan-2-yl)isoxazol-3-yl]-3-[3-(6,7-dimethoxyquinazolin-4-ylthio)phenyl]urea;
1-[3-(6,7-dimethoxyquinazolin-4-ylthio)phenyl]-3-[1-phenyl-3-(trifluoromethyl)-1H-pyrazol-5-yl]urea;
1-[3-(6,7-dimethoxyquinazolin-4-ylthio)phenyl]-3-[1-methyl-3-(trifluoromethyl)-1H-pyrazol-5-yl]urea;
1-[3-(6,7-dimethoxyquinazolin-4-ylthio)phenyl]-3-[1-methyl-5-(trifluoromethyl)-1H-pyrazol-3-yl]urea;
ethyl 2-(3-tert-butyl-5-{3-[3-(6,7-dimethoxyquinazolin-4-ylthio)phenyl]ureido}-1H-pyrazol-1-yl)acetate;
1-[3-(1,3-difluoro-2-methylpropan-2-yl)-1-phenyl-1H-pyrazol-5-yl]-3-[3-(6,7-dimethoxyquinazolin-4-ylthio)phenyl]urea;
1-[3-(6,7-dimethoxyquinazolin-4-ylthio)phenyl]-3-[3-(2-ethoxypropan-2-yl)-1-phenyl-1H-pyrazol-5-yl]urea;
1-[3-(6,7-dimethoxyquinazolin-4-ylthio)phenyl]-3-[1-(4-fluorophenyl)-3-(trifluoromethyl)-1H-pyrazol-5-yl]urea;
1-[3-(6,7-dimethoxyquinazolin-4-ylthio)phenyl]-3-[1-p-tolyl-3-(trifluoromethyl)-1H-pyrazol-5-yl]urea;
1-(3-(6,7-dimethoxyquinazolin-4-ylthio)phenyl)-3-(3-(2-methoxyethoxy)-5-(trifluoromethyl)phenyl)urea;
1-(5-cyclopentylisoxazol-3-yl)-3-(3-(6,7-dimethoxyquinazolin-4-ylthio)phenyl)urea;
1-(3-tert-butylisoxazol-5-yl)-3-(3-(6-methoxy-7-(2-methoxyethoxy)quinazolin-4-yloxy)phenyl)urea;
1-(3-(6-Methoxy-7-(2-methoxyethoxy)quinazolin-4-yloxy)phenyl)-3-(5-phenylisoxazol-3-yl)urea;
1-(3-(6-methoxy-7-(2-methoxyethoxy)quinazolin-4-yloxy)phenyl)-3-(3-(morpholine-4-carbonyl)-5-(trifluoromethyl)phenyl)urea;
1-(5-isopropylisoxazol-3-yl)-3-(3-(6-methoxy-7-(2-methoxyethoxy)quinazolin-4-yloxy)phenyl)urea;
1-(3-cyclopentylisoxazol-5-yl)-3-(3-(6-methoxy-7-(2-methoxyethoxy)quinazolin-4-yloxy)phenyl)urea;
1-{3-[6-methoxy-7-(2-methoxyethoxy)quinazolin-4-yloxy]phenyl}-3-[1-methyl-5-(trifluoromethyl)-1H-pyrazol-3-yl]urea;
1-(3-tert-butyl-1-methyl-1H-pyrazol-5-yl)-3-(3-(6-methoxy-7-(2-methoxyethoxy)quinazolin-4-yloxy)phenyl)urea;
1-(3-tert-butyl-1-phenyl-1H-pyrazol-5-yl)-3-(3-(6-methoxy-7-(2-methoxyethoxy)quinazolin-4-yloxy)phenyl)urea;
1-(3-(1,1-difluoroethyl)isoxazol-5-yl)-3-(3-(6-methoxy-7-(2-methoxyethoxy)quinazolin-4-yloxy)phenyl)urea;
1-[3-(2-ethoxypropan-2-yl)-1-phenyl-1H-pyrazol-5-yl]-3-{3-[6-methoxy-7-(2-methoxyethoxy)quinazolin-4-yloxy]phenyl}urea;
1-[5-(1,3-difluoro-2-methylpropan-2-yl)isoxazol-3-yl]-3-{3-[6-methoxy-7-(2-methoxyethoxy)quinazolin-4-yloxy]phenyl}urea;
1-(3-cyclopropylisoxazol-5-yl)-3-(3-(6-methoxy-7-(2-methoxyethoxy)quinazolin-4-yloxy)phenyl)urea;
1-(3-isopropylisoxazol-5-yl)-3-(3-(6-methoxy-7-(2-methoxyethoxy)quinazolin-4-yloxy)phenyl)urea;
1-(3-(6-methoxy-7-(2-methoxyethoxy)quinazolin-4-yloxy)phenyl)-3-(3-(tetrahydro-2H-pyran-4-yl)isoxazol-5-yl)urea;
1-(3-(2-fluoropropan-2-yl)isoxazol-5-yl)-3-(3-(6-methoxy-7-(2-methoxyethoxy)quinazolin-4-yloxy)phenyl)urea;
1-(5-cyclopentylisoxazol-3-yl)-3-(3-(6-methoxy-7-(2-methoxyethoxy)quinazolin-4-yloxy)phenyl)urea;
1-{3-[6-methoxy-7-(2-methoxyethoxy)quinazolin-4-yloxy]phenyl}-3-[1-methyl-3-(trifluoromethyl)-1H-pyrazol-5-yl]urea;
1-{3-[6-methoxy-7-(2-methoxyethoxy)quinazolin-4-yloxy]phenyl}-3-[1-phenyl-3-(trifluoromethyl)-1H-pyrazol-5-yl]urea;
1-(3-fluoro-4-(trifluoromethyl)phenyl)-3-(3-(6-methoxy-7-(2-methoxyethoxy)quinazolin-4-yloxy)phenyl)urea;
1-(3-methoxy-4-(trifluoromethyl)phenyl)-3-(3-(6-methoxy-7-(2-methoxyethoxy)quinazolin-4-yloxy)phenyl)urea;
ethyl 2-[3-tert-butyl-5-(3-{3-[6-methoxy-7-(2-methoxyethoxy)quinazolin-4-yloxy ]phenyl}ureido)-1H-pyrazol-1-yl]acetate hydrochloride;
1-{3-[6-methoxy-7-(2-methoxyethoxy)quinazolin-4-yloxy]phenyl}-3-[1-phenyl-5-(trifluoromethyl)-1H-pyrazol-3-yl]urea;
1-[1-(4-fluorophenyl)-3-(trifluoromethyl)-1H-pyrazol-5-yl]-3-{3-[6-methoxy-7-(2-methoxy ethoxy)quinazolin-4-yloxy]phenyl}urea;
1-{3-[6-methoxy-7-(2-methoxyethoxy)quinazolin-4-yloxy]phenyl}-3-[1-p-tolyl-3-(trifluoromethyl)-1H-pyrazol-5-yl]urea;
1-[3-(1,3-difluoro-2-methylpropan-2-yl)-1-phenyl-1H-pyrazol-5-yl]-3-{3-[6-methoxy-7-(2-methoxyethoxy)quinazolin-4-yloxy]phenyl}urea;
1-(3-(6-methoxy-7-(2-methoxyethoxy)quinazolin-4-yloxy)phenyl)-3-(3-(trifluoromethyl)isoxazol-5-yl)urea;
1-[5-(1,3-difluoro-2-methylpropan-2-yl)isoxazol-3-yl]-3-{3-[6-methoxy-7-(2-methoxyethoxy)quinazolin-4-ylthio]phenyl}urea;
1-(3-fluoro-4-(trifluoromethyl)phenyl)-3-(3-(6-methoxy-7-(2-methoxyethoxy)quinazolin-4-ylthio)phenyl)urea;
1-(5-isopropylisoxazol-3-yl)-3-(3-(6-methoxy-7-(2-methoxyethoxy)quinazolin-4-ylthio)phenyl)urea;
1-(3-methoxy-4-(trifluoromethyl)phenyl)-3-(3-(6-methoxy-7-(2-methoxyethoxy)quinazolin-4-ylthio)phenyl)urea;
1-(3-(2-fluoropropan-2-yl)isoxazol-5-yl)-3-(3-(6-methoxy-7-(2-methoxyethoxy)quinazolin-4-ylthio)phenyl)urea;
1-(5-cyclopentylisoxazol-3-yl)-3-(3-(6-methoxy-7-(2-methoxyethoxy)quinazolin-4-ylthio)phenyl)urea;
1-(3-tert-butyl-1-phenyl-1H-pyrazol-5-yl)-3-(3-(6-methoxy-7-(2-methoxyethoxy)quinazolin-4-ylthio)phenyl)urea;
ethyl 2-[3-tert-butyl-5-(3-{3-[6-methoxy-7-(2-methoxyethoxy)quinazolin-4-ylthio]phenyl}ureido)-1H-pyrazol-1-yl]acetate;
1-[3-(1,3-difluoro-2-methylpropan-2-yl)-1-phenyl-1H-pyrazol-5-yl]-3-{3-[6-methoxy-7-(2-methoxyethoxy)quinazolin-4-ylthio]phenyl}urea;
1-{3-[6-methoxy-7-(2-methoxyethoxy)quinazolin-4-ylthio]phenyl}-3-[1-methyl-3-(trifluoromethyl)-1H-pyrazol-5-yl]urea;
1-{3-[6-methoxy-7-(2-methoxyethoxy)quinazolin-4-ylthio]phenyl}-3-[1-methyl-3-(trifluoromethyl)-1H-pyrazol-5-yl]urea;
1-[3-(2-methoxypropan-2-yl)-1-phenyl-1H-pyrazol-5-yl]-3-{3-[6-methoxy-7-(2-methoxyethoxy)quinazolin-4-ylthio]phenyl}urea;
1-[1-(4-fluorophenyl)-3-(trifluoromethyl)-1H-pyrazol-5-yl]-3-{3-[6-methoxy-7-(2-methoxyethoxy)quinazolin-4-ylthio]phenyl}urea;
l-{3-[6-methoxy-7-(2-methoxyethoxy)quinazolin-4-ylthio]phenyl}-3-[1-p-tolyl-3-(trifluoromethyl)-1H-pyrazol-5-yl]urea;
1-{3-[6-methoxy-7-(2-methoxyethoxy)quinazolin-4-ylthio]phenyl}-3-[1-phenyl-5-(trifluoromethyl)-1H-pyrazol-3-yl]urea;
1-(3-(2-fluoropropan-2-yl)isoxazol-5-yl)-3-(3-(7-methoxy-6-(4,4-dioxo-3-thiomorpholinopropoxy)quinazolin-4-ylthio)phenyl)urea;
1-(4-methoxy-3-(trifluoromethyl)phenyl)-3-(3-(7-methoxy-6-(3-(4,4-dioxothiomorpholino)propoxy)quinazolin-4-ylthio)phenyl)urea;
1-(3-(6,7-bis(2-Methoxyethoxy)quinazolin-4-ylthio)phenyl)-3-(3-tert-butylisoxazol-5-yl)urea;
1-(3-(2-fluoropropan-2-yl)isoxazol-5-yl)-3-(3-(6-methoxy-7-(2-morpholinoethoxy)quinazolin-4-ylthio)phenyl)urea;
1-(4-methoxy-3-(trifluoromethyl)phenyl)-3-(3-(6-methoxy-7-(2-morpholinoethoxy) quinazolin-4-ylthio)phenyl)urea;
1-(4-methoxy-3-(trifluoromethyl)phenyl)-3-(3-(6-methoxy-7-(2-morpholinoethoxy)quinazolin-4-yloxy)phenyl)urea;
1-(3-(2-fluoropropan-2-yl)isoxazol-5-yl)-3-(3-(6-methoxy-7-(2-morpholinoethoxy)quinazolin-4-yloxy)phenyl)urea;
1-(1-tert-butyl-1H-pyrazol-4-yl)-3-(3-(6,7-dimethoxyquinazolin-4-yloxy)phenyl)urea;
1-(5-tert-butylisoxazol-3-yl)-3-(3-(6,7-dimethoxyquinazolin-4-ylsulfinyl)phenyl)urea;
1-(3-(6,7-dimethoxyquinazolin-4-yloxy)phenyl)-3-(3-(trifluoromethyl)isoxazol-5-yl)urea;
1-(3-(6,7-dimethoxyquinazolin-4-yloxy)phenyl)-3-(3-(1-hydroxy-2-methylpropan-2-yl)isoxazol-5-yl)urea;
1-(5-tert-butyl-isoxazol-3-yl)-3-(3-{7-[3-(1,1-dioxo-thiomorpholin-4-yl)-propoxy]-6-methoxy-quinazolin-4-yloxy}-phenyl)-urea;
1-(3-(2-fluoropropan-2-yl)isoxazol-5-yl)-3-(3-(7-hydroxy-6-methoxyquinazolin-4-yloxy)phenyl)urea;
1-(3-(2-fluoropropan-2-yl)isoxazol-5-yl)-3-(3-(6-hydroxy-7-methoxyquinazolin-4-yloxy)phenyl)urea;
1-(3-(6,7-dimethoxyquinazolin-4-yloxy)phenyl)-3-(5-(1,1,1-trifluoro-2-methylpropan-2-yl)isoxazol-3-yl)urea;
1-(3-(6-ethoxy-7-methoxyquinazolin-4-yloxy)phenyl)-3-(5-(1,1,1-trifluoro-2-methylpropan-2-yl)isoxazol-3-yl)urea;
1-(3-(6,7-dimethoxyquinazolin-4-ylthio)phenyl)-3-(5-(1,1,1-trifluoro-2-methylpropan-2-yl)isoxazol-3-yl)urea;
1-(3-(6-ethoxy-7-methoxyquinazolin-4-ylthio)phenyl)-3-(5-(1,1,1-trifluoro-2-methylpropan-2-yl)isoxazol-3-yl)urea;
1-(3-(7-hydroxy-6-methoxyquinazolin-4-yloxy)phenyl)-3-(5-(1,1,1-trifluoro-2-methylpropan-2-yl)isoxazol-3-yl)urea;
1-(3-(6-hydroxy-7-methoxyquinazolin-4-yloxy)phenyl)-3-(5-(1,1,1-trifluoro-2-methylpropan-2-yl)isoxazol-3-yl)urea;
1-(3-(6,7-dimethoxyquinazolin-4-yloxy)-2-fluorophenyl)-3-(5-(1,1,1-trifluoro-2-methylpropan-2-yl)isoxazol-3-yl)urea;
1-(3-(6,7-dimethoxyquinazolin-4-yloxy)-4-fluorophenyl)-3-(5-(1,1,1-trifluoro-2-methylpropan-2-yl)isoxazol-3-yl)urea;
1-(3-(6,7-dimethoxyquinazolin-4-yloxy)phenyl)-3-(3-(1,1,1-trifluoro-2-methylpropan-2-yl)isoxazol-5-yl)urea;
1-(3-(6,7-dimethoxyquinazolin-4-ylthio)phenyl)-3-(3-(1,1,1-trifluoro-2-methylpropan-2-yl)isoxazol-5-yl)urea; and
1-(5-(6,7-dimethoxyquinazolin-4-yloxy)-2,4-difluorophenyl)-3-(3-(2-fluoropropan-2 yl)isoxazol-5-yl)urea;
or a pharmaceutically acceptable salt or prodrug, thereof.

In some embodiments, the compound is selected from:
1-(5-tert-butylisoxazol-3-yl)-3-(5-(6,7-dimethoxyquinazolin-4-yloxy)-2,4-difluorophenyl)urea;
1-(5-(6,7-dimethoxyquinazolin-4-yloxy)-2,4-difluorophenyl)-3-(1-phenyl-3-(trifluoromethyl)-1H-pyrazol-5-yl)urea;
1-(3-tert-butyl-1-p-tolyl-1H-pyrazol-5-yl)-3-(5-(6,7-dimethoxyquinazolin-4-yloxy)-2,4-difluorophenyl)urea;
1-(3-tert-butyl-1-phenyl-1H-pyrazol-5-yl)-3-(5-(6,7-dimethoxyquinazolin-4-yloxy)-2,4-difluorophenyl)urea;
1-(3-tert-butyl-1-p-tolyl-1H-pyrazol-5-yl)-3-(3-(6,7-dimethoxyquinazolin-4-yloxy)phenyl)urea;
1-(3-tert-butyl-1-p-tolyl-1H-pyrazol-5-yl)-3-(3-(6,7-dimethoxyquinazolin-4-ylthio)phenyl)urea;
1-(3-tert-butyl-1-p-tolyl-1H-pyrazol-5-yl)-3-(3-(6,7-methoxy-7-(2-methoxyethoxy)quinazolin-4-yloxy)phenyl)urea;
1-(3-tert-Butyl-1-p-tolyl-1H-pyrazol-5-yl)-3-(3-(6-methoxy-7-(2-methoxyethoxy)quinazolin-4-ylthio)phenyl)urea;
1-(3-(2-cyanopropan-2-yl)phenyl)-3-(3-(6,7-dimethoxyquinazolin-4-yloxy)phenyl)urea;
1-(3-(2-cyanopropan-2-yl)phenyl)-3-(3-(6,7-dimethoxyquinazolin-4-ylthio)phenyl)urea;
1-(3-(2-cyanopropan-2-yl)phenyl)-3-(3-(6-methoxy-7-(2-methoxyethoxy)quinazolin-4-yloxy)phenyl)urea;
1-(3-(2-cyanopropan-2-yl)phenyl)-3-(3-(6-methoxy-7-(2-methoxyethoxy)quinazolin-4-ylthio)phenyl)urea;
1-(3-tert-butyl-1-(2,4-dimethylphenyl)-1H-pyrazol-5-yl)-3-(3-(6,7-dimethoxyquinazolin-4-yloxy)phenyl)urea;
1-(3-tert-butyl-1-(2,4-dimethylphenyl)-1H-pyrazol-5-yl)-3-(3-(6-methoxy-7-(2-methoxyethoxy)quinazolin-4-ylthio)phenyl)urea;
1-(3-tert-butyl-1-(2,4-dimethylphenyl)-1H-pyrazol-5-yl)-3-(3-(6,7-dimethoxyquinazolin-4-ylthio)phenyl)urea;
1-(3-tert-butyl-1-m-tolyl-1H-pyrazol-5-yl)-3-(3-(6,7-dimethoxyquinazolin-4-yloxy)phenyl)urea;
1-(3-tert-butyl-1-m-tolyl-1H-pyrazol-5-yl)-3-(3-(6,7-dimethoxyquinazolin-4-ylthio)phenyl)urea;
1-(3-tert-butyl-1-m-tolyl-1H-pyrazol-5-yl)-3-(3-(6-methoxy-7-(2-methoxyethoxy)quinazolin-4-ylthio)phenyl)urea;
1-(3-tert-butyl-1-p-tolyl-1H-pyrazol-5-yl)-3-(3-(6,7-dimethoxyquinazolin-4-yloxy)-2-methylphenyl)urea;
1-(3-tert-butyl-1-phenyl-1H-pyrazol-5-yl)-3-(3-(6,7-dimethoxyquinazolin-4-yloxy)-2-methylphenyl)urea;
1-(5-tert-butylisoxazol-3-yl)-3-(3-(6,7-dimethoxyquinazolin-4-yloxy)-2-methylphenyl)urea;
1-(3-(6,7-Dimethoxyquinazolin-4-yloxy)-2-methylphenyl)-3-(1-phenyl-3-(trifluoromethyl)-1H-pyrazol-5-yl)urea;
l-(3-(6,7-dimethoxyquinazolin-4-yloxy)-2-methylphenyl)-3-(5-(2-fluoropropan-2-yl)isoxazol-3-yl)urea;
1-(3-(6,7-dimethoxyquinazolin-4-yloxy)-4-fluorophenyl)-3-(3-(2-fluoropropan-2-yl)isoxazol-5-yl)urea;
1-(5-(1,3-difluoro-2-methylpropan-2-yl)isoxazol-3-yl)-3-(3-(6,7-dimethoxyquinazolin-4-yloxy)-4-fluorophenyl)urea;
1-(3-tert-butyl-1-phenyl-1H-pyrazol-5-yl)-3-(3-(6,7-dimethoxyquinazolin-4-yloxy)-2-fluorophenyl)urea;
1-(3-(6-dimethoxyquinazolin-4-yloxy)-2-fluorophenyl)-3-(3-(2-fluoropropan-2-yl)isoxazol-5-yl)urea;
1-(5-(1,3-difluoro-2-methylpropan-2-yl)isoxazol-3-yl)-3-(3-(6,7-dimethoxyquinazolin-4-yloxy)-2-fluorophenyl)urea;
1-(3-tert-butyl-1-p-tolyl-1H-pyrazol-5-yl)-3(2-chloro-5-(6,7-dimethoxyquinazolin-4-yloxy)phenyl)urea;
1-(5-tert-butylisoxazol-3-yl)-3-(2-chloro-5-(6,7-dimethoxyquinazolin-4-yloxy)phenyl)urea;
1-(2-Chloro-5-(6,7-dimethoxyquinazolin-4-yloxy)phenyl)-3-(1-phenyl-3-(trifluoromethyl)-1H-pyrazol-5-yl)urea;
1-(3-tert-butyl-1-phenyl-1H-pyrazol-5-yl)-3-(2-chloro-5-(6,7-dimethoxyquinazolin-4-yloxy)phenyl)urea;
1-(3-(6,7-dimethoxyquinazolin-4-yloxy)phenyl)-3-(5-(2-methyl-1-morpholinopropan-2-yl)isoxazol-3-yl)urea;
1-(3-(3-tert-butyl-1-(4-methylpyridin-3-yl)-1H-pyrazol-5-yl)-3-(3-(6,7-dimethoxyquinazolin-4-yloxy)phenyl)urea;
1-(3-tert-butyl-1-(pyridin-3-yl)-1H-pyrazol-5-yl)-3-(3-(6,7-dimethoxyquinazolin-4-yloxy)phenyl)urea;
1-(3-(6,7-dimethoxyquinazolin-4 -yloxy)phenyl)-3-(3-(perfluoroethyl)-1-phenyl-1H-pyrazol-5-yl)urea;
1-(3-tert-butyl-1-(2-methylpyridin-3-yl)-1H-pyranzol-5-yl)-3-(3-(6,7-dimethoxyquinazolin-4-yloxy)phenyl)urea;
1-(3-(6,7-dimethoxyquinazolin-4-yloxy)phenyl)-3-(1-phenyl-3-(1,1,1-trifluoro-2-methylpropan-2-yl)-1H-pyrazol-5-yl)urea;
1-(3-(6,7-dimethoxyquinazolin-4-ylthio)phenyl)-3-(1-phenyl-3-(1,1,1-trifluoro-2-methylpropan-2-yl)-1H-pyrazol-5-yl)urea;
1-(3-(2-cyanopropan-2-yl)-1-phenyl-1H-pyrazol-5-yl)-3-(3-(6,7-dimethoxyquinazolin-4-yloxy)phenyl)urea;
1-(3-(2-cyanopropan-2-yl)-1-phenyl-1H-pyrazol-5-yl)-3-(3-(6,7-dimethoxyquinazolin-4-ylthio) phenyl)urea;
1-(5-tert-butylisoxazol-3-yl)-3-(3-(2-chloro-6,7-dimethoxyquinazolin-4-yloxy)phenyl)urea;
1-(3-(1,1-difluoroethyl)-1-(pyridin-3-yl)-1H-pyrazol-5-yl)-3-(3-(6,7-dimethoxyquinazolin-4-yloxy)phenyl)urea;
1-(3-tert-butyl-1-(6-methylpyridin-3-yl)-1H-pyrazol-5-yl)-3-(3-(6,7-dimethoxyquinazolin-4-yloxy)phenyl)urea;
1-(3-tert-butyl-1-(2-oxo-1,2-dihydropyridin-4-yl)-1H-pyrazol-5-yl)-3-(3-(6,7-dimethoxyquinazolin-4-yloxy)phenyl)urea;
1-(3-(6,7-dimethoxyquinazolin-4-yloxy)phenyl)-3-(1-(5-fluoropyridin-3-yl)-3-isopropyl-1H-pyrazol-5-yl)urea;
1-(3-(1,1-difluoroethyl)-1-(4-methoxypheny)-1H-pyrazol-5-yl)-3-(3-(6,7-dimethoxyquinazolin-4-yloxy)phenyl)urea;
1-(3-(1,1-difluoroethyl)-1-(5-fluoropyridin-3-yl)-1H-pyrazol-5-yl)-3-(3-(6,7-dimethoxyquinazolin-4-yloxy)phenyl)urea;
1-(3-tert-butyl-1-(6-oxo-1,6-dihydropyridin-3-yl)-1H-pyrazol-5-yl)-3-(3-(6,7-dimethoxyquinazolin-4-yloxy)phenyl)urea;
1-(3-(1,1-difluoroethyl)-1-phenyl-1H-pyrazol-5-yl)-3-(3-(6,7-dimethoxyquinazolin-4-yloxy)phenyl)urea;
1-(3-(1,1-difluoroethyl)-1-phenyl-1H-pyrazol-5-yl)-3-(3-(6,7-dimethoxyquinazolin-4-ylthio)phenyl)urea;
1-(3-tert-butyl-1-(2-methylpyridin-4-yl)-1H-pyrazol-5-yl)-3-(3-(6,7-dimethoxyquinazolin-4-yloxy)phenyl)urea;
1-(3-tert-butyl-1-ethyl-1H-pyrazol-5-yl)-3-(3-(6,7-dimethoxyquinazolin-4-ylthio)phenyl)urea;
1-(3-tert-butyl-1-(pyridin-3-yl)-1H-pyrazol-5-yl)-3-(3-(6,7-dimethoxyquinazolin-4-yloxy)phenyl)urea;
1-(3-tert-butyl-1-(pyridin-3-yl)-1H-pyrazol-5-yl)-3-(3-(6,7-dimethoxyquinazolin-4-ylthio)phenyl)urea;
1-(3-(6,7-dimethoxyquinazolin-4-yloxy)phenyl)-3-(3-isopropyl-1-phenyl-1H-pyrazol-5-yl)urea;
1-(3-(6,7-dimethoxyquinazolin-4-ylthio)phenyl)-3-(3-isopropyl-1-phenyl-1H-pyrazol-5-yl)urea;
1-(3-tert-butyl-1-(5-fluoropyridin-3-yl)-1H-pyrazol-5-yl)-3-(3-(6,7-dimethoxyquinazolin-4-yloxy)phenyl)urea;
1-(3-tert-butyl-1-(5-fluoropyridin-3-yl)-1H-pyrazol-5-yl)-3-(3-(6,7-dimethoxyquinazolin-4-ylthio)phenyl)urea;
1-(3-tert-butyl-1-(4-cyanophenyl)-1H-pyrazol-5-yl)-3-(3-(6,7-dimethoxyquinazolin-4-yloxy)phenyl)urea;
1-(3-tert-butyl-1-(4-cyanophenyl)-1H-pyrazol-5-yl)-3-(3-(6,7-dimethoxyquinazolin-4-ylthio)phenyl)urea;
1-(3-tert-butyl-1-cyclohexyl-1H-pyrazol-5-yl)-3-(3-(6,7-dimethoxyquinazolin-4-yloxy)phenyl)urea;
1-(3-tert-butyl-1-cyclohexyl-1H-pyrazol-5-yl)-3-(3-(6,7-dimethoxyquinazolin-4-ylthio)phenyl)urea;
1-(3-tert-butyl-1-isobutyl-1H-pyrazol-5-yl)-3-(3-(6,7-dimethoxyquinazolin-4-yloxy)phenyl)urea;
1-(3-tert-butyl-1-isobutyl-1H-pyrazol-5-yl)-3-(3-(6,7-dimethoxyquinazolin-4-ylthio)phenyl)urea;
1-(3-tert-butyl-1-isopropyl-1H-pyrazol-5-yl)-3-(3-(6,7-dimethoxyquinazolin-4-yloxy)phenyl)urea;
1-(3-tert-butyl-1-isopropyl-1H-pyrazol-5-yl)-3-(3-(6,7-dimethoxyquinazolin-4-ylthio)phenyl)urea;
1-(3-tert-butyl-1-(pyridin-4-1H-pyrazol-5-yl)-3-(3-(6,7-dimethoxyquinazolin-4-yloxy)phenyl)urea;
1-(3-tert-butyl-1-(pyridin-4-yl)-1H-pyrazol-5-yl)-3-(3-(6,7-dimethoxyquinazolin-4-ylthio)phenyl)urea;
1-(3-(6,7-dimethoxyquinazolin-4-yloxy)phenyl)-3-(1-m-tolyl-3-(trifluoromethyl)1H-pyrazol-5-yl)urea;
1-(3-(6,7-dimethoxyquinazolin-4-ylthio)phenyl)-3-(1-m-tolyl-3-(trifluoromethyl)-1H-pyrazol-5-yl)urea;
1-(3-tert-butyl-1-(2-chlorophenyl)-1H-pyrazol-5-yl)-3-(3-(6,7-dimethoxyquinazolin-4-yloxy)phenyl)urea;
1-(3-tert-butyl-1-(2-chlorophenyl)-1H-pyrazol-5-yl)-3-(3-(6,7-dimethoxyquinazolin-4-ylthio)phenyl)urea;
1-(3-tert-butyl-1-o-tolyl-1H-pyrazol-5-yl)-3-(3-(6,7-dimethoxyquinazolin-4-yloxy)phenyl)urea;
1-(3-tert-butyl-1-o-tolyl-1H-pyrazol-5-yl)-3-(3-(6,7-dimethoxyquinazolin-4-ylthio)phenyl)urea;
1-(3-tert-butyl-1-(pyridin-2-yl)-1H-pyrazol-5-yl)-3-(3-(6,7-dimethoxyquinazolin-4-yloxy)phenyl)urea;
1-(3-tert-butyl-1-(pyridin-2-yl)-1H-pyrazol-5-yl)-3-(3-(6,7-dimethoxyquinazolin-4-ylthio)phenyl)urea;
1-(3-(6,7-dimethoxyquinazolin-4-yloxy)phenyl)-3-(1-p-tolyl-3-(1-(trifluoromethyl)cyclopropyl)-1H-pyrazol-5-yl)urea;
1-(3-(6,7-dimethoxyquinazolin-4-ylthio)phenyl)-3-(1-p-tolyl-3-(1-(trifluoromethyl)cyclopropyl)-1H-pyrazol-5-yl)urea;
1-(3-(6,7-dimethoxyquinazolin-4-yloxy)phenyl)-3-(3-isopropyl-1-(4-methoxyphenyl)-1H-pyrazol-5-yl)urea;
1-(3-(6,7-dimethoxyquinazolin-4-ylthio)phenyl)-3-(3-isopropyl-1-(4-methoxyphenyl)-1H-pyrazol-5-yl)urea;
1-(3-(6,7-dimethoxyquinazolin-4-yloxy)phenyl)-3-(3-isopropyl-1-(pyridin-3-yl)-1H-pyrazol-5-yl)urea;
1-(3-(6,7-dimethoxyquinazolin-4-ylthio)phenyl)-3-(3-isopropyl-1-(pyridin-3-yl)-1H-pyrazol-5-yl)urea;
1-(3-(6,7-dimethoxyquinazolin-4-yloxy)phenyl)-3-(3-ethyl-1-phenyl-1H-pyrazol-5-yl)urea;
1-(3-cyclopropyl-1-phenyl-1H-pyrazol-5-yl)-3-(3-(6,7-dimethoxyquinazolin-4-yloxy)phenyl)urea;
1-(3-cyclopropyl-1-phenyl-1H-pyrazol-5-yl)-3-(3-(6,7-dimethoxyquinazolin-4-ylthio)phenyl)urea;
1-(3-cyclobutyl-1-phenyl-1H-pyrazol-5-yl)-3-(3-(6,7-dimethoxyquinazolin-4-yloxy)phenyl)urea;
1-(3-cyclobutyl-1-phenyl-1H-pyrazol-5-yl)-3-(3-(6,7-dimethoxyquinazolin-4-ylthio)phenyl)urea;
1-(1-benzyl-3-tert-butyl-1H-pyrazol-5-yl)-3-(3-(6,7-dimethoxyquinazolin-4-yloxy)phenyl)urea;
1-(1-benzyl-3-tert-butyl-1H-pyrazol-5-yl)-3-(3-(6,7-dimethoxyquinazolin-4-ylthio)phenyl)urea;
1-(3-tert-butyl-1-(3-fluorophenyl)-1H-pyrazol-5-yl)-3-(3-(6,7-dimethoxyquinazolin-4-yloxy)phenyl)urea;
1-(3-tert-butyl-1-(3-fluorophenyl)-1H-pyrazol-5-yl)-3-(3-(6,7-dimethoxyquinazolin-4-ylthio)phenyl)urea;
1-(3-tert-butyl-1-(4-methoxyphenyl)-1H-pyrazol-5-yl)-3-(3-(6,7-dimethoxyquinazolin-4-yloxy)phenyl)urea;
1-(3-tert-butyl-1-(4-methoxyphenyl)-1H-pyrazol-5-yl)-3-(3-(6,7-dimethoxyquinazolin-4-ylthio)phenyl)urea;
1-(3-tert-butyl-1-(3-chlorophenyl)-1H-pyrazol-5-yl)-3-(3-(6,7-dimethoxyquinazolin-4-yloxy)phenyl)urea;
1-(3-tert-butyl-1-(3-chlorophenyl)-1H-pyrazol-5-yl)-3-(3-(6,7-dimethoxyquinazolin-4-ylthio)phenyl)urea;
1-(3-tert-butyl-1-(4-chlorophenyl)-1H-pyrazol-5-yl)-3-(3-(6,7-dimethoxyquinazolin-4-yloxy)phenyl)urea;
1-(3-tert-butyl-1-(4-chlorophenyl)-1H-pyrazol-5-yl)-3-(3-(6,7-dimethoxyquinazolin-4-ylthio)phenyl)urea;
1-(5-tert-butylisoxazol-3-yl)-3-(5-(6,7-dimethoxyquinazolin-4-yloxy)-2-fluorophenyl)urea;
1-(3-tert-butyl-1-phenyl-1H-pyrazol-5-yl)-3-(5-(6,7-dimethoxyquinazolin-4-yloxy)-2-fluorophenyl)urea;
1-(3-tert-butyl-1-(4-tert-butylphenyl)-1H-pyrazol-5-yl)-3-(3-(6,7-dimethoxyquinazolin-4-yloxy)phenyl)urea;
1-(3-tert-butyl-1-(4-tert-butylphenyl)-1H-pyrazol-5-yl)-3-(3-(6,7-dimethoxyquinazolin-4-ylthio)phenyl)urea;
1-(3-tert-butyl-1-(2-fluorophenyl)-1H-pyrazol-5-yl)-3-(3-(6,7-dimethoxyquinazolin-4-ylthio)phenyl)urea;
1-(3-tert-butyl-1-(2-fluorophenyl)-1H-pyrazol-5-yl)-3-(3-(6,7-dimethoxyquinazolin-4-yloxy)phenyl)urea;
1-(3-tert-butyl-1-(4-(trifluoromethyl)phenyl)-1H-pyrazol-5-yl)-3-(3-(6,7-dimethoxyquinazolin-4-yloxy)phenyl)urea;
1-(3-tert-butyl-1-(4-(trifluoromethyl)phenyl)-1H-pyrazol-5-yl)-3-(3-(6,7-dimethoxyquinazolin-4-ylthio)phenyl)urea;
1-(3-tert-butyl-1-(2-(trifluoromethyl)phenyl)-1H-pyrazol-5-yl)-3-(3-(6,7-dimethoxyquinazolin-4-ylthio)phenyl)urea;
1-(3-tert-butyl-1-(2-(trifluoromethyl)phenyl)-1H-pyrazol-5-yl)-3-(3-(6,7-dimethoxyquinazolin-4-yloxy)phenyl)urea;
1-(3-(6,7-dimethoxyquinazolin-4-yloxy)phenyl)-3-(5-(1-(trifluoromethyl)cyclopropyl)isoxazol-3-yl)urea;
1-(3-(6,7-dimethoxyquinazolin-4-ylthio)phenyl)-3-(5-(1-(trifluoromethyl)cyclopropyl)isoxazol-3-yl)urea;
1-(3-tert-butyl-1-(3-(trifluoromethyl)phenyl)-1H-pyrazol-5-yl)-3-(3-(6,7-dimethoxyquinazolin-4-yloxy)phenyl)urea;
1-(3-tert-butyl-1-(3-(trifluoromethyl)phenyl)1H-pyrazol-5-yl)-3-(3-(6,7-dimethoxyquinazolin-4-ylthio)phenyl)urea;
1-(3-(2-cyanopropan-2-yl)isoxazol-5-yl)-3-(3-(6,7-dimethoxyquinazolin-4-ylthio)phenyl)urea;
1-(3-(6,7-dimethoxyquinazolin-4-ylthio)phenyl)-3-(3-(trifluoromethyl)isoxazol-5-yl)urea;
1-(3-(6,7-dimethoxyquinazolin-4-ylthio)phenyl)-3-(1-phenyl-3-(1-(trifluoromethyl)cyclopropyl)-1H-pyrazol-5-yl)urea;
1-(3-(7-ethoxy-6-methoxyquinazolin-4-yloxy)phenyl)-3-(1-phenyl-3-(trifluoromethyl)-1H-pyrazol-5-yl)urea;
1-(3-(7-ethoxy-6-methoxyquinazolin-4-yloxy)phenyl)-3-(3-(2-fluoropropan-2-yl)isoxazol-5-yl)urea;
1-(5-(1,3-difluoro-2-methylpropan-2-yl)isoxazol-3-yl)-3-(3-(7-ethoxy-6-methoxyquinazolin-4-yloxy)phenyl)urea;
1-(3-tert-butyl-1-phenyl-1H-pyrazol-5-yl)-3-(3-(7-ethoxy-6-methoxyquinazolin-4-yloxy)phenyl)urea;
1-(5-(1,3-difluoro-2-methylpropan-2-yl)isoxazol-3-yl)-3-(3-(7-ethoxy-6-methoxyquinazolin-4-ylthio)phenyl)urea;
1-(3-(7-ethoxy-6-methoxyquinazolin-4-ylthio)phenyl)-3-(3-(2-fluoropropan-2-yl)isoxazol-5yl)urea;
1-(3-tert-butyl-1-phenyl-1H-pyrazol-5-yl)-3-(3-(6-ethoxy-7-methoxyquinazolin-4-yloxy)phenyl)urea;
1-(3-(6-ethoxy-7-methoxyquinazolin-4-yloxy)phenyl)-3-(1-phenyl-3-(trifluoromethyl)-1H-pyrazol-5-yl)urea;
1-(3-(6-ethoxy-7-methoxyquinazolin-4-yloxy)phenyl)-3-(3-(2-fluoropropan-2-yl)isoxazol-5-yl)urea;
1-(5-(3-difluoro-2-methylpropan-2-yl)isoxazol-3-yl)-3-(3-(6-ethoxy-7-methoxyquinazolin-4-yloxy)phenyl)urea;
1-(3-(6-ethoxy-7-methoxyquinazolin-4-ylthio)phenyl)-3-(3-(2-fluoropropan-2-yl)isoxazol-5-yl)urea;
1-(5-(1,3-difluoro-2-methylpropan-2-yl)isoxazol-3-yl)-3-(3-(6-ethoxy-7-methoxyquinazolin-4-ylthio)phenyl)urea;
(1-(3-(6-methoxy-7-(2-morpholinoethoxy)quinazolin-4-yloxy)phenyl)-3-(1-phenyl-3-(trifluoromethyl)-1H-pyrazol-5-yl)urea;
1-(3-(6,7-Dimethoxyquinazolin-4-yloxy)-4-fluorophenyl)-3-(1-phenyl-3-(trifluoromethyl)-1H-pyrazol-5-yl)urea;
1-(3-tert-butyl-1-(4-methoxyphenyl)-1H-pyrazol-5-yl)-3-(3-(6,7-dimethoxyquinazolon-4-yloxy)phenyl)urea; and
1-(3-(6,7-dimethoxyquinazolin-4-ylthio)phenyl)-3-(3-ethyl-1-phenyl-1H-pyrazol-5-yl)urea
or a pharmaceutically acceptable salt or prodrug thereof.

In some embodiments, the RAF inhibitor is-CEP32496:

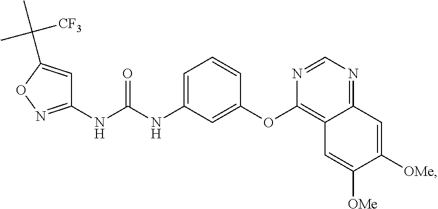 [SMILES: COc1cc2ncnc(Oc3cccc(NC(=O)Nc4cc(C(C)(C)C(F)(F)F)on4)c3)c2cc1OC]

or a pharmaceutically acceptable salt thereof.

(iv) PCT Application Publication WO 2008/147782, which is incorporated herein by reference:

Substituted benzimidazole compounds having the following formula D-I:

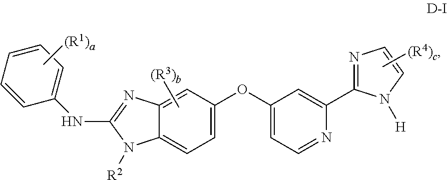

wherein each R¹ is independently selected from hydroxy, halo, C₁₋₆alkyl, C₁₋₆alkoxy, (C₁₋₆alkyl)sulfanyl, (C₁₋₆alkyl)sulfonyl, cycloalkyl, heterocycloalkyl, phenyl and heteroaryl;
R² is C₁₋₆alkyl or halo(C₁₋₆-alkyl);
each R³ is independently selected from halo, C₁₋₆alkyl and C₁₋₆alkoxy;
each R⁴ is independently selected from hydroxy, C₁₋₆alkyl, C₁₋₆alkoxy, halo, heterocycloalkylcarbonyl, carboxyl, (C₁₋₆alkoxy)carbonyl, aminocarbonyl, C₁₋₆alkylaminocarbonyl, carbonitrile, cycloalkyl, heterocycloalkyl, phenyl and heteroaryl;
wherein R¹, R², R³ and R⁴ may be optionally substituted with one or more substituents independently selected from hydroxy, halo, C₁₋₆alkyl, halo(C₁₋₆alkyl), C₁₋₆alkoxy and halo(C₁₋₆alkoxy);
a is 1, 2, 3, 4 or 5;
b is 0, 1, 2 or 3; and
c is 1 or 2;
or a tautomer, stereoisomer, ester, or prodrug thereof or a pharmaceutically acceptable salt of the compound, tautomer, stereoisomer, ester, or prodrug.

In some embodiments, the compound has a structure of formula D-II:

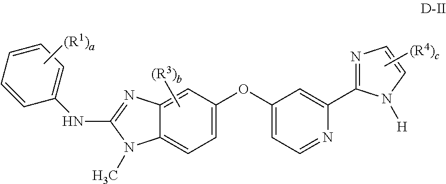

wherein each R¹ is independently selected from C₁₋₆alkyl, C₁₋₆alkoxy, hydroxy, halo, (C₁₋₆alkyl)sulfanyl, (C₁₋₆alkyl)sulfonyl, cycloalkyl, heterocycloalkyl, phenyl and heteroaryl;
each R³ is independently selected from halo, C₁₋₆alkyl and C₁₋₆alkoxy;
each R⁴ is independently selected from hydroxy, C₁₋₆alkyl, C₁₋₆alkoxy, halo, carboxyl, (C₁₋₆alkoxy)carbonyl, aminocarbonyl carbonitrile, cycloalkyl, heterocycloalkyl, heterocycloalkylcarbonyl, phenyl and heteroaryl;
wherein R¹, R², R³ and R⁴ may be optionally substituted with one or more substituents independently selected from hydroxy, halo, C₁₋₆alkyl and C₁₋₆alkoxy;
a is 1, 2, 3, 4 or 5;
b is 0, 1, 2 or 3; and
c is 1 or 2;
or a tautomer, stereoisomer, ester, or prodrug thereof or a pharmaceutically acceptable salt of the compound, tautomer, stereoisomer, ester, or prodrug.

In some embodiments, the compound has a structure of formula D-III:

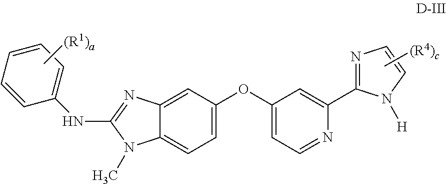

wherein each R¹ is independently selected from C₁₋₆alkyl, C₁₋₆alkoxy, hydroxy, halo, (C₁₋₆alkyl)sulfanyl, (C₁₋₆alkyl)sulfonyl, cycloalkyl, heterocycloalkyl, phenyl, and heteroaryl;
each R⁴ is independently selected from hydroxy, C₁₋₆alkyl, C₁₋₆alkoxy, halo, carboxyl, (C₁₋₆alkoxy)carbonyl, aminocarbonyl, carbonitrile, cycloalkyl, heterocycloalkyl, heterocycloalkylcarbonyl, phenyl and heteroaryl;
wherein R¹ and R⁴ may be optionally substituted with one or more substituents independently selected from hydroxy, halo, C₁₋₆alkyl and C₁₋₆alkoxy;
a is 1, 2, 3, 4 or 5; and
c is 1 or 2;
or a tautomer, stereoisomer, ester, or prodrug thereof or a pharmaceutically acceptable salt of the compound, tautomer, stereoisomer, ester, or prodrug.

Also disclosed are compounds of the following formula D-IV:

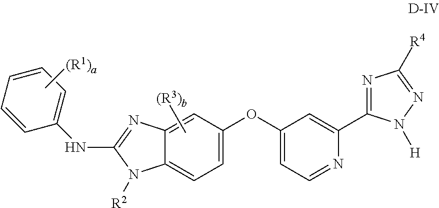

wherein each R¹ is independently selected from C₁₋₆alkyl, C₁₋₆alkoxy, hydroxy, halo, (C₁₋₆alkyl)sulfanyl, (C₁₋₆alkyl)sulfonyl, cycloalkyl, heterocycloalkyl, phenyl and heteroaryl;
R² is C₁₋₆alkyl or halo(C₁₋₆alkyl);
each R³ is independently selected from halo, C₁₋₆alkyl and C₁₋₆alkoxy;
each R⁴ is independently selected from hydroxy, C₁₋₆alkyl, C₁₋₆alkoxy, halo, carboxyl, (C₁₋₆alkoxy)carbonyl, aminocarbonyl, C₁₋₆alkylaminocarbonyl, carbonitrile, carbonitrile(C₁₋₆alkyl), cycloalkyl, heterocycloalkyl, heterocycloalkyl(C₁₋₆alkyl), heterocycloalkylcarbonyl, phenyl and heteroaryl;
wherein R¹, R², R³ and R⁴ may be optionally substituted with one or more substituents independently selected from hydroxy, halo, C₁₋₆alkyl and C₁₋₆alkoxy;
a is 1, 2, 3, 4 or 5; and
b is 0, 1, 2 or 3;
or a tautomer, stereoisomer, ester, or prodrug thereof or a pharmaceutically acceptable salt of the compound, tautomer, stereoisomer, ester, or prodrug.

In other embodiments, new substituted benzimidazole compounds are provided of formulae D-I through D-IV set forth above, wherein each R¹ is independently selected from the group consisting of hydroxy, chloro, fluoro, bromo, methyl, ethyl, propyl, butyl, methoxy, ethoxy, propoxy, butoxy, trifluoromethyl, trifluoroethyl, trifluoromethoxy, trifluoroethoxy, trifluoromethylsulfanyl piperidinyl C₁₋₆alkylpiperidinyl, piperazinyl, C₁₋₆alkylpiperazinyl, tetrahydrofuranyl, pyridinyl and pyrimidinyl. In other embodiments, new substituted benzimidazole compounds are provided of formulae D-I through D-IV, wherein a is 1 or 2, and at least one R¹ is halo(C₁₋₆alkyl), such as trifluoromeythyl. In other embodiments, new substituted benzimidazole compounds are provided of formulae D-I and D-IV, wherein R² is C₁₋₆alkyl, such as, e.g., methyl or ethyl. In further embodiments, new substituted benzimidazole compounds are provided of formulae D-I, D-II and D-IV, wherein b is 0, and thus R³ is not present. In alternate embodiments, new substituted benzimidazole compounds are provided of formulae D-I through D-IV, wherein b is 1, and R³ is C₁₋₆alkoxy, such as, e.g., methoxy. In yet further embodiments, new substituted benzimidazole compounds are provided of formulae D-I through D-IV, wherein b is 1 or 2, and at least one R⁴ is halo(C₁₋₆alkyl), such as, e.g., trifluoromethyl.

In some embodiments, the compound is Raf-265:

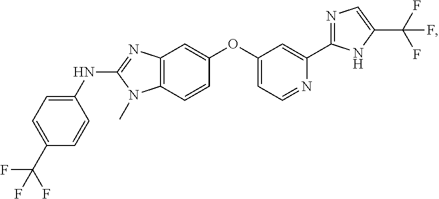 [SMILES: Cn1c(Nc2ccc(C(F)(F)F)cc2)nc2cc(Oc3ccnc(-c4ncc(C(F)(F)F)[nH]4)c3)ccc21]

or a pharmaceutically acceptable salt thereof.

(v) PCT Application Publication WO 02/24680, which is incorporated herein by reference:

A compound of formula E-I:

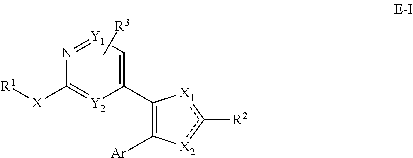

wherein
X is O, CH₂, CO, S or NH, or the moiety X-R¹ is hydrogen;
Y₁ and Y₂ are independently N or CH;
R¹ is hydrogen, C₁₋₆alkyl, C₃₋₇cycloalkyl, aryl, arylC₁₋₆alkyl, heterocyclyl, heterocyclylC₁₋₆alkyl, heteroaryl, or heteroarylC₁₋₆alkyl any of which may be optionally substituted; in addition when X is CH₂ then R¹ may be hydroxyl or C₁₋₆alkoxy which may be optionally substituted;
R² is H, C₁₋₆alkyl, C₂₋₆alkenyl, C₃₋₇cycloalkyl, C₅₋₇cycloalkenyl, heterocyclyl, aryl or heteroaryl, any of which may be optionally substituted;
Ar is s group of the formula a) or b);

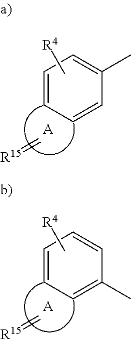

wherein A represents a fused 5- to 7-membered ring optionally containing up to two heteroatoms selected from O, S and NR⁵, wherein R⁵ is hydrogen or C₁₋₆alkyl, which ring is optionally substituted by up to 2 substituents selected from halogen, C₁₋₆alkyl, hydroxy, C₁₋₆alkoxy or keto;
R³ and R⁴ are independently selected from hydrogen, halogen, C₁₋₆alkyl, aryl, aryl C₁₋₆alkyl, C₁₋₆alkoxy, C₁₋₆alkoxyC₁₋₆alkyl, haloC₁₋₆alkyl, arylC₁₋₆alkoxy, hydroxy, nitro, cyano, azido, amino, mono- and di-N-C₁₋₆alkylamino, acylamino, arylcarbonylamino, acyloxy, carboxy, carboxy salts, carboxy esters, carbamoyl, mono- and di-N-C₁₋₆alkylcarbamoyl, C₁₋₆alkoxycarbonyl, aryloxycarbonyl, ureido, guanidino, C₁₋₆alkylguanidino, amidino, C₁₋₆alkylamidino, sulphonylamino, aminosulphonyl, C₁₋₆alkylthio, C₁₋₆alkylsulphinyl or C₁₋₆alkylsulphonyl;
R¹⁵ is O or N—OH;
one of X₁ and X₂ is N and the other is NR⁶ wherein R⁶ is hydrogen or C₁₋₆alkyl; or a pharmaceutically acceptable salt thereof.

In some embodiments, the compound is SB-590885:

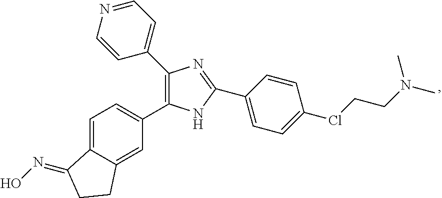

or a pharmaceutically acceptable salt thereof.

Method of Treating HCV-infection with the Administration of a RAF Inhibitor or a Pharmaceutically Acceptable Salt Thereof

In one aspect, the invention provides methods for treating HCV-infection in a subject in need thereof by administering a RAF kinase inhibitor or a pharmaceutically acceptable salt thereof. The RAF kinase inhibitor suitable for the method of this invention may be selected from any of those as described above. In some embodiments, the compound suitable for use in the methods of the present invention is an inhibitor of one or more RAF kinases selected from A-RAF, B-RAF, and C-RAF. In some embodiments, the RAF inhibitor is selected from the group of compounds referred to in U.S. Pat. No. 7,235,576 and U.S. Pat. No. 7,351,834, and International Patent Application Publications WO 02/24680, WO 2006/024834, WO 2008/147782, and WO 2009/117080 (each of the U.S. Patent or PCT publication is incorporated herein in its entirety by reference for their disclosure of RAF inhibitors). In certain embodiments of the invention, the RAF inhibitor is selected from sorafenib, CEP32496, Raf-265, regorafenib, SB-590885, AZ-628, or prodrugs or pharmaceutically acceptable salts thereof. In some embodiments, the RAF inhibitor is selected from sorafenib, Raf-265, and AZ-628.

In accordance with this invention, the RAF inhibitors and pharmaceutically acceptable salts thereof can be administered to a subject via any sultable route or routes. In some embodiments, the drugs are administered orally; however, administration intravenously, subcutaneously, intra-arterially, intramuscularly, intraspinally, rectally, intrathoracically, intraperitoneally, intraventricularly, or transdermally, topically, or by inhalation is also contemplated. The agents can be administered orally, for example, in the form of tablets, troches, capsules, elixirs, suspensions, syrups, wafers, or the like, prepared by art recognized procedures.

In some embodiments, the administration is a slow or extended release. The term “extended release” is widely recognized in the art of pharmaceutical sciences and is used herein to refer to a controlled release of an active compound or agent from a dosage form to an environment over (throughout or during) an extended period of time, e.g. greater than or equal to one hour. An extended release dosage form will release drug at substantially constant rate over an extended period of time or a substantially constant amount of drug will be released incrementally over an extended period of time. The. term “extended release” used herein includes the terms “controlled release,” “prolonged release,” “sustained release,” “delayed release,” or “slow release” as these terms are used in the pharmaceutical sciences. In some embodiments, the extended release dosage is administered in the form of a patch or a pump.

When a solid carrier is used for administration, the preparation may be in a tablet, placed in a hard gelatin capsule in powder or pellet form, or it may be in the form of a troche or lozenge. If a liquid carrier is used, the preparation may be in the forms of a syrup, emulsion, soft gelatin capsule, or sterile injectable liquid such as an aqueous or non-aqueous liquid suspension or solution.

Dosage schedules of the agents and compositions according to the methods of the invention will vary according to the particular compound or compositions selected, the route of administration, the nature of the condition being treated, the age, and condition of the patient, the course, or stage of treatment, and will ultimately be at the discretion of the attending physician. It will be understood that the amount of the RAF inhibitor and their pharmaceutically acceptable salts thereof administered will be amounts effective to produce a desired biological effect such as beneficial results, including clinical results. It will be understood that an effective amount can be administered in more than one dose and over a course of treatment.

Desired duration of administration of the RAF inhibitor and their pharmaceutically acceptable salts thereof can be determined by routine experimentation by one skilled in the art. For example, the RAF inhibitor and/or its pharmaceutically acceptable salts may be administered for a period of 1-4 weeks, 1-3 months, 3-6 months, 6-12 months, 1-2 years, or more, up to the lifetime of the patient. In some embodiments, the RAF inhibitor and/or its pharmaceutically acceptable salts may be administered for a period of 1 week to 48 weeks. In some embodiments, the RAF inhibitor and their pharmaceutically acceptable salts may be administered for a period of 12 weeks or 24 weeks.

It is known in the art that normalization to body surface area is an appropriate method for extrapolating doses between species. The human equivalent dose (HED) for this dosage can be estimated using the following formula that accounts for differences in body surface area (see Estimating the Safe Starting Dose in Clinical Trials for Therapeutics in Adult Healthy Volunteers, December 2002, Center for Biologies Evaluation and Research):

HED=animal dose×(Km animal/Km human)

where the Km factor is body weight divided by body surface area (Km rat has been determined as 6, and Km human is 37; see Reagan-Saw, Nihal, Ahmad, 2007). Thus, a dosage of 10 mg/kg in rats is equivalent to 1.6 mg/kg in humans (10 mg/kg×(6/37)=1.6 mg/kg). For human subjects, to calculate a dose in mg from the dose in mg/kg, the dose in mg/kg is multiplied by a typical adult weight of 70 kg.

In certain embodiments of the invention, the dose of the RAF inhibitor or its pharmaceutically acceptable salt is 0.01 to 10 mg/kg/day (which, given a typical human subject of 70 kg, is 0.7 to 700 mg/day).

In accordance with another aspect of the present invention, there is provided a method for treating HCV infection in a subject in need thereof, the method comprising the step of administering to said subject an RAF inhibitor or a pharmaceutically acceptable salt thereof in combination with at least one additional therapeutic agent. In some embodiments, the additional therapeutic agent is an antiviral agent. In some embodiments, the additional therapeutic agent is a current, late stage or marketed DAA agent. Suitable antiviral agents include, but are not limited to, HCV protease and polymerase inhibitors (such as NS3/4A protease inhibitors and RNA-dependent RNA polymerase (NS5B) inhibitors), agents targeting host cell activities involved in HCV replication and inosine monophosphate dehydrogenase (IMPDH) inhibitors.

Examples of therapeutic agents that may be present in a combination include ribavirin, levovirin, viramidine, thymosin alpha-1, interferon-β, interferon-α, pegylated interferon-α (peginterferon-a), a combination of interferon-α and ribavirin, a combination of peginterferon-α and ribavirin, a combination of interferon-α and levovirin, and a combination of peginterferon-α and levovirin. Interferon-α includes recombinant interferon-α2a (such as ROFERON interferon available from Hoffmann-LaRoche, Nutley, N.J.), pegylated interferon-α2a (PEGASYS), interferon-α2b (such as INTRON-A interferon available from Schering Corp., Kenilworth, N.J.), pegylated interferon-α2b (PEGINTRON), a recombinant consensus interferon (such as interferon alphacon-1), and a purified interferon-α product. Amgen's recombinant consensus interferon has the brand name INFERGEN. Levovirin is the L-enantiomer of ribavirin which has shown immunomodulatory activity similar to ribavirin. Viramidine represents an analog of ribavirin disclosed in WO 01/60379. The individual components of the combination can be administered separately at different times during the course of therapy or concurrently in divided or single combination forms.

Ribavirin, levovirin, and viramidine may exert their anti-HCV effects by modulating intracellular pools of guanine nucleotides via inhibition of the intracellular enzyme inosine monophosphate dehydrogenase (IMPDH). IMPDH is the rate-limiting enzyme on the biosynthetic route in de novo guanine nucleotide biosynthesis. Ribavirin, is readily phosphorylated intracellularly and the monophosphate derivative is an inhibitor of IMPDH. Thus, inhibition of IMPDH represents another useful target for the discovery of inhibitors of HCV replication. Therefore, the compounds of the present invention may also be administered in combination with an inhibitor of IMPDH, such as VX-497, which is disclosed in International Patent Application Publications WO 97/41211 and WO 01/09622; another IMPDH inhibitor, such as that disclosed in WO 09/25780; or mycophenolate mofetil. See A. C. Allison and E. M. Eugui, 44 (Suppl.) Agents Action 165 (1993).

Macrocyclic compounds useful as HCV protease inhibitors are described in WO 06/119061, WO 7/015785, WO 7/016441, WO 07/148135, WO 08/051475, WO 08/051477, WO 08/051514, WO 08/057209. Some HCV NS3 protease inhibitors are disclosed in International Patent Application Publications WO 98/22496, WO 98/46630, WO 99/07733, WO 99/07734, WO 99/38888, WO 99/50230, WO 99/64442, WO 00/09543, WO 00/59929, WO 02/48116, WO 02/48172, British Patent No. GB 2 337 262, and U.S. Pat. No. 6,323,180.

Hepatitis C NS3/4A protease inhibitor may be employed in the present disclosure as the additional therapeutic agent. A compound may be assayed for its ability to inhibit Hepatitis C protease by methods known in the art and/or by methods provided herein. Examples of such inhibitors include, but are not limited to, compounds identified as inhibitors in such assays and the inhibitors of WO 03/087092, WO 03/006490, WO 03/064456, WO 03/064416, WO 03/035060, WO 02/060926, WO 02/079234, WO 02/48116, WO 02/48157, WO 00/31129, WO 02/18369, WO 02/08256, WO 02/08244, WO 02/08198, WO 02/08187, WO 01/81325, WO 01/77113, WO 01/74768, WO 01/64678, WO 01/07407, WO 00/59929, WO 00/09588, WO 00/09543, WO 99/64442, WO 99/50230, WO 99/38888, WO 99/07734, WO 99/07733, WO 98/46630, WO 98/46630, WO 98/22496, WO 98/17679, WO 97/43310, U.S. Pat. No. 6,018,020, U.S. Pat. No. 5,990,276, U.S. Pat. No. 5,866,684, U.S.20030008828, U.S.20020177725, U.S.20020016442, U.S.20020016294, M. Llinas-Drunet et al, Bioorg. Med. Chem. Lett., 8, pp. 1713-18 (1998); W, Han et al., Bioorg. Med. Chem, Lett, 10,711-13 (2000); R. Dunsdon et al, Bioorg. Med. Chem. Lett., 10, pp. 1571-79 (2000); M. Llinas-Brunet et al, Bioorg Med, Chem. Lett., 10, pp. 2267-70 (2000); and S. LaPlante et al., Bioorg. Med. Chem. Lett, 10, pp. 2271-74 (2000)] (which are incorporated herein by reference).

HCV NS5B polymerase inhibitors that may be used in combination with the RAF inhibitors in the present disclosure include, but are not limited to, those disclosed in International Patent Application Publications WO 02/057287, WO 02/057425, WO 03/068244, WO 2004/000858, WO 04/003138 and WO 2004/007512; U.S. Pat. No. 6,777,392 and U.S. Patent Application Publication U.S.2004/0067901; the content of each is incorporated herein by reference in its entirety. Other such HCV polymerase inhibitors include, but are not limited to, valopicitabine (NM-283; Idenix) and 2′-F-2′-beta-methylcytidine (see also WO 2005/003147). In some embodiments, nucleoside HCV NS5B polymerase inhibitors that are used in combination with the present RAF inhibitors are selected from the following compounds:
4-amino-7-(2-C-methyl-β-D-arabinofuranosyl)-7H-pyrrolo[2,3-d]pyrimidine;
4-amino-7-(2-C-methyl-β-D-ribofuranosyl)-7H-pyrrolo[2,3-d]pyrimidine;
4-methylamino-7-(2-C-methyl-β-D-ribofuranosyl(-7H-pyrrolo[2,3-d]pyrimidine;
4-dimethylamino-7-(2-C-methyl-β-D-ribofuranosyl(-7H-pyrrolo[2,3-d]pyrimidine;
4-cyclopropylamino-7-(2-C-methyl-β-D-ribofuranosyl)-7H-pyrrolo[2,3-d]pyrimidine;
4-amino-7-(2-C-vinyl-β-D-ribofuranosyl)-7H-pyrrolo[2,3-d]pyrimidine;
4-amino-7-(2-C-hydroxymethyl-β-D-ribofuranosyl)-7H-pyrrolo[2,3-d]pyrimidine;
4-amino-7-(2-C-fluoromethyl-β-D-ribofuranosyl)-7H-pyrrolo[2,3-d]pyrimidine;
4-amino-5-methyl-7-(2-C-methyl-β-D-ribofuranosyl)-7H-pyrrolo[2,3-d]pyrimidine;
4-amino-7-(2-C-methyl-β-D-ribofuranosyl)-7H-pyrrolo[2,3-d]pyrimidine-5-carboxylic acid;
4-amino-5-bromo-7-(2-C-methyl-β-D-ribofuranosyl)-7H-pyrrolo[2,3-d]pyrimidine;
4-amino-5-chloro-7-(2-C-methyl-β-D-ribofuranosyl)-7H-pyrrolo[2,3-d]pyrimidine;
4-amino-5-fluoro-7-(2-C-methyl-β-D-ribofuranosyl)-7Hpyrrolo[2,3-d]pyrimidine;
2,4-diamino-7-(2-C-methyl-β-D-ribofuranosyl)-7H-pyrrolo[2,3-d]pyrimidine;
2-amino-7-(2-C-methyl-β-D-ribofuranosyl)-7H-pyrrolo[2,3-d]pyrimidine;
2-amino-4-cyclopropylamino-7-(2-C-methyl-β-D-ribofuranosyl)-7H-pyrrolo[2,3-d]pyrimidine;
2-amino-7-(2-C-methyl-β-D-ribofuranosyl)-7H pyrrolo[2,3-d]pyrimidin-4(3H)-one;
4-amino-7-(2-C-ethyl-β-D-ribofuranosyl)-7H-pyrrolo[2,3-d]pyrimidine;
4-amino-7-(2-C,2-O-dimethyl-β-D-ribofuranosyl)-7H-pyrrolo[2,3-d]pyrimidine;
7-(2-C-methyl-β-D-ribofuranosyl)-7H-pyrrolo[2,3-d]pyrimidine-4(3H)-one;
2-amino-5-methyl-7-(2-C,2-O-dimethyl-β-D-ribofuranosyl)-7H-pyrrolo[2,3-d]pyrimidin-4(3H)-one;
4-amino-7-(3-deoxy-2-C-methyl-β-D-ribofuranosyl)-7H-pyrrolo[2,3-d]pyrimidine;
4-amino-7-(3-deoxy-2-C-methyl-β-D-arabinofuranosyl)-7H-pyrrolo[2,3-d]pyrimidine;
4-amino-2-fluoro-7-(2-C-methyl-β-D-ribofuranosyl)-7H-pyrrolo[2,3-d]pyrimidine;
4-amino-7-(3-C-methyl-β-D-ribofuranosyl)-7H-pyrrolo[2,3-d]pyrimidine;
4-amino-7-(3-C-methyl-β-D-xylofuranosyl)-7H-pyrrolo[2,3-d]pyrimidine;
4-amino-7-(2,4-di-C-methyl-β-D-ribofuranosyl)-7H-pyrrolo[2,3-d]pyrimidine;
4-amino-7-(3-deoxy-3-fluoro-2-C-methyl-β-D-ribofuranosyl)-7H-pyrrolo[2,3-d]pyrimidine; and the corresponding 5′-triphosphates; or a pharmaceutically acceptable salt thereof.

In certain embodiments, the additional therapeutic agent is GS-7977 (an nucleotide HCV NS5B polymerase inhibitor):

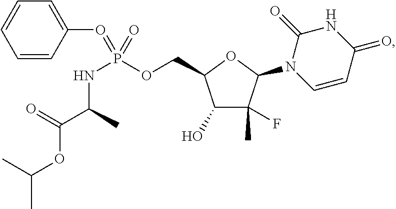

or pharmaceutically acceptable salts thereof. In certain embodiments, the additional therapeutic agent is BMS-790052 (a NS5A inhibitor):

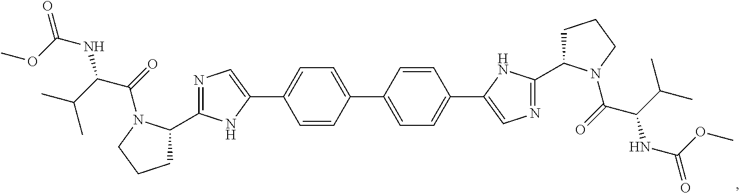

or a pharmaceutically acceptable salt thereof. In certain embodiments, the method of the present disclosure comprises administering to a subject in need thereof a therapeutically effective amount of AZ-628 conjointly with GS-7977 or BMS-790052.

In some embodiments of the invention, the RAF inhibitor and the additional therapeutic agent are administered simultaneously, or sequentially, or in a single formulation or in separate formulations packaged together. In other embodiments, the RAF inhibitor and the additional therapeutic agent are administered via different routes. As used herein, “combination” includes administration by any of these formulations or routes of administration.

It will be understood by one of ordinary skill in the art that the methods described herein may be adapted and modified as is appropriate for the application being addressed and that the methods described herein may be employed in other suitable applications, and that such other additions and modifications will not depart from the scope hereof.

This invention will be better understood from the Experimental Details which follow. However, one skilled in the art will readily appreciate that the specific methods and results discussed are merely illustrative of the invention as described more fully in the embodiments which follow thereafter.

### EXAMPLES

### Example 1

### RAF Inhibitors

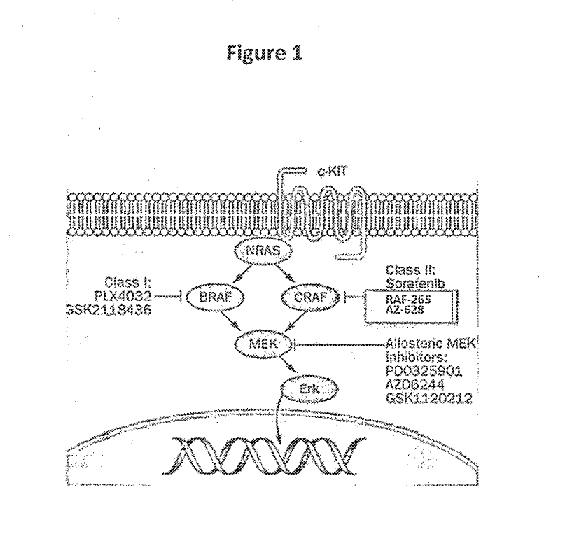

To identify potential compounds that could be advanced as part of a comprehensive anti-HCV therapy, a series of compounds that inhibit one or more RAF isoforms were evaluated. The tested inhibitors are shown in bold in FIG. 1.

The intrinsic ability of these RAF inhibitors to block one or more RAF isoforms is characterized in Table 1.

TABLE 1
Kinase Profile of RAF inhibitors to be evaluated as antiviral agents

| | | | IC50 | | | |
|---|---|---|---|---|---|---|
| | | IC50 | B-Raf | IC50 | IC50 | IC50 |
| | | C-Raf | (wt) | VEGFR2 | c-Kit | FLT3 |
| Drug | | (nmol/ | (nmol/ | (nmol/ | (nmol/ | (nmol/ |
| Substance | Class^{a} | L) | L) | L) | L) | L) |
| sorafenib | II | 6 | 22 | 90 | 68 | 58 |
| Raf265 | II | 5^{b} | 70^{b} | 70^{b} | 20^{b} | NA^{b} |
| Zelboraf/ | I | 1.72 | 0.16 | —^{c} | —^{c} | —^{c} |
| PLX4032 | | | | | | |
| AZ-628 | II | 29 | 100 | —^{b} | —^{b} | —^{b} |
| GSK2118436/ | I | 48 | 100 | —^{c} | —^{c} | —^{c} |
| dabrafinib | | | | | | |

^{a}Class I inhibitors have cellular selectivity for B-Raf mutations associated with melanoma, such as V600E; such inhibitors bind wildtype B-Raf with equal proficiency to mutant protein in purified form, but selectively target the mutant protein when present inside cells. Class II inhibitors do not discriminate mutant B-Raf from their wildtype counterpart.
^{b}Not available.
^{c}No meaningful inhibition.

### Example 2

### Antiviral Potency of Raf Inhibitors against HCVpp

### Overview of Experimental Design

The initial screen for anti-HCV activity utilized HCV pseudoparticles (HCVpp), the equivalent of an HCV-VLP, to evaluate the effect of Raf inhibition on viral entry. The HCV entry inhibitor assay uses Huh 7.5 cells and HIV-1 virus which has been pseudotyped with the HCV (GT1a or GT1b) E1/E2. Briefly, cells are plated one day prior to assay. On the day of assay, media is aspirated and 50 μl of 2× compound and 50 μl of pretitered virus are added to each well. Toxicity plates receive only the test compound in tissue culture medium. On day 5, the medium is removed from all wells of the efficacy plates, followed by one wash with medium and addition of 100 ∥l of firefly luciferase reagent and subsequent luciferase detection is carried out using a Microbeta detector (Wallac). For toxicity plates, 10 μl of MTS reagent is added to all wells and incubated until cell control optical density values are between 1 and 2. Compound cytotoxicity is assessed by MTS (CellTiter®96 Reagent, Promega, Madison Wis.) dye reduction. The % reduction in viral entry is determined and reported). Each assay includes 1 μMg/ml cyanovirin and/or 10 μg/ml antibody CD81 as a positive control.

### Cell Preparation

Huh 7.5 cells (human hepatocyte cell line) were obtained from APath LLC and were grown in Dulbecco's Modified Eagle's Medium (DMEM) supplemented with 5% fetal bovine serum (FBS), 2.0 mM L-Glutamine, 100 units/ml Penicillin and 100 μg/ml Streptomycin, and 0.1 mM non-essential amino acids (“growth medium”). Cells were sub-cultured twice a week at a split ratio of 1:15 using standard cell culture techniques. Total cell number and percent viability determinations were performed using a hemacytometer and trypan blue exclusion. Cell viability must be greater than 95% for the cells to be utilized in the assay. The cells are seeded in 96-well tissue culture plates the day before the assay at a concentration of 8-10×10 cells/well.

### Virus Preparation

The virus used for this assay is HIV-1 where the HIV envelope is knocked out and then pseudotyped with the HCV (GT1a or GT1b) E1/E2. This virus also has a luciferase reporter gene. For each assay, a pre-titered aliquot of virus is removed from the freezer (−80° C.) and allowed to thaw slowly to room temperature in a biological safety cabinet. The virus is resuspended and diluted into tissue culture medium such that the amount of virus added to each well is at a MOI (multiplicity of infection) of 1.

### Plate Formal

The format of the test plate has been standardized. Each efficacy plate contains cell control wells (cells only), virus control wells (cells plus virus), and experimental wells (drug plus cells plus virus). Toxicity plate wells contain cell control wells (cells only) and experimental wells (cells plus drug in the absence of virus). Samples were evaluated for antiviral efficacy with triplicate measurements using 6 concentrations at half-log (or other) dilutions (12 concentrations can also be performed) in order to determine EC₅₀ values and cellular cytotoxicity, if detectable. Compounds were tested at 6 concentrations using a representative high-test concentration of 100 μM.

### Results of the Viral Inhibitory Assay

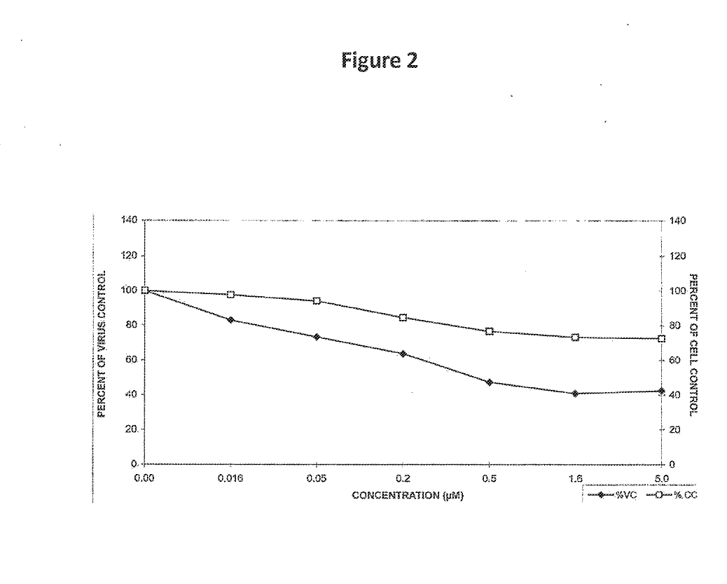

The results of the anti-HCV assay using HCVpp constructed from genotype 1b (GT1b) are shown in Table 2. A graphical representation of the dose response of compound AZ-628 on HCVpp viral entry into cultured human hepatocytes is shown in FIG. 2.

TABLE 2
Antiviral potency against HCV entry using virus-like
particles in Huh 7.5.1

| Drug | Genotype | EC50 (μM)¹ | TI² | Class³ |
|---|---|---|---|---|
| Sorafenib | 1b | 1.81 | >2.8 (5 μM) | II |
| Raf-265 | 1b | 0.89 | >5.6 (5 μM) | II |
| AZ-628 | 1b | 0.42 | >49 (20 μM) | II |
| Zelboraf/PLX4032 | 1b | >5 | 1 (5 μM) | I |
| Dabrafenib/ | 1b | >5 | 1 (5 μM) | I |
| GSK2118436 | | | | |

¹Measured by luciferase tagged HCV GT-1b pseudotyped virus in ≧80% confluent cells at MOI 0.02.
²Ratio of EC₅₀ and CC₅₀, where EC₅₀ is the concentration necessary to inhibit viral reproduction by 50% and CC₅₀ is the intrinsic drug cytotoxicity measured in the absence of virus. Number in parentheses indicates the highest concentration tested in the experiment. > sign indicates that concentration range tested was too low to fully evaluate the TI, so the lower limit is specified.
³Defined by the specificity of the inhibitor to a RAF isoform.

The date suggest a potent entry blockage for Class II Raf inhibitors. Class I inhibitors show little or no antiviral activity.

### Example 3

### Antiviral Potency of RAF Inhibitors against HCVcc

### Overview of Experimental Design

The secondary screen, for anti-HCV activity utilized HCVcc, a lab-adapted full infection cycle virus, to evaluate the effect of Raf inhibition on viral entry. The HCVcc inhibitor assay uses Huh 7.5 cells and HCVcc containing the luciferase gene generated from pJ6/JFH1-p7Rluc. Briefly cells are plated one day prior to assay. On the day of assay, media is aspirated. The cells are pre-treated with 100 μl of 1X compound. After 1 h pretreatment, compound containing media is aspirated, HCVcc diluted 1x compound is added to each well. Toxicity plates receive only the test compound in tissue culture medium. The medium is removed from all wells of the efficacy plate 72 h post-infection, followed one wash with 1xPBS and addition of 30 μl of Renilla luciferase reagent and subsequent luciferase detection using a Microbeta detector (Wallac). For toxicity plates, 10 μl of MTS reagent is added to all wells and incubated until cell control optical density values are between 1 and 2. Compound cytotoxicity is assessed by MTS (CellTiter® 96 Reagent, Promega, Madison Wis.) dye reduction. The % reduction in viral entry is determined and reported. Each assay includes anti-CD81 as a positive control.

### Cell Preparation

Huh 7.5 cells (human hepatocyte cell line) were obtained from APath LLC and were grown in Dulbecco's Modified Eagle's Medium (DMEM) supplemented with 10% fetal bovine serum (FBS), 100 units/ml Penicillin and 100 μg/ml Streptomycin, 1% Non-essential amino acids(NEAA) in a 5% CO₂ incubator at 37° C. Cells were sub-cultured twice a week at a split ratio of 1:10 using standard cell culture techniques. Total cell number and percent viability determinations were performed using a hemacytometer and trypan blue exclusion. Cell viability must be greater than 95% for the cells to be utilized in the assay. The cells were seeded in 96-well tissue culture plates the day before the assay at a concentration of 1×10⁴ cells/well.

Generation of Viral stocks and HCVcc Titration

The plasmids carrying HCV J6/JFH1 genome, or chimeric mCon1/JFH1 or mH77/JFH1 genome and Rluc reporter gene were kindly provided by Dr. Charles Rice at Rockefeller University. HCV RNA was produced by in vitro transcription using a RiboMax™ Large scale RNA production system (Promega) according to manufacturer's instructions. HCV RNA was delivered to Huh7.5.1 cells by using TranslT mRNA transfection kit (Mirus). The infectious supernatants were harvested from 72 h-192 h post-transfection and filtered through a 0.22 μm membrane. The cleared supernatant (HCVcc) was aliquoted and frozen at −80° C. or used freshly. HCVcc infectivity was determined by TCID₅₀ assay. Briefly, infectious supernatant was series diluted and inoculated onto Huh7.5 cells in a 96-well plate at 37° C. At 72 h post-infection, cells were fixed and immunostained with the anti-NS5A MAb. The number of HCV positive cells was counted. The TCID₅₀ was calculated according to the Reed and Muench method.

### Plate Format

The format of the test plate has been standardized by SOUTHERN. Each efficacy plate contains cell control wells (cells only), virus-control wells (cells plus virus), and experimental wells (drug plus cells plus virus). Toxicity plate wells contain cell control wells (cells only) and experimental wells (cells plus drug in the absence of virus). Samples were evaluated for antiviral efficacy with triplicate measurements using 6 concentrations at half-log (or other) dilutions in order to determine EC₅₀ values and cellular cytotoxicity, if detectable. Compounds were tested at 6 concentrations using a representative high-test concentration of 100 μM.

### Results of the Viral Inhibitory Assay

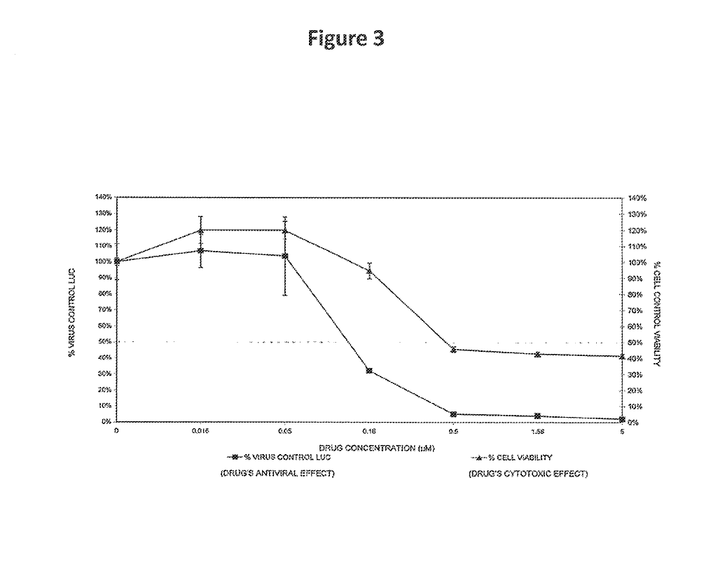

Table 4 illustrates the potency of antiviral inhibition against HCVcc. A graphical representation of the dose response of compound AZ-628 on HCVcc viral entry into cultured human hepatocytes is shown in FIG. 3.

TABLE 3
Antiviral potency against HCVcc in Huh 7.5.1

| Drug | Genotype | EC50 (μM)¹ | TI² | Class³ |
|---|---|---|---|---|
| Raf-265 | 2a | 1.05 | >3.7 (5 μM) | II |
| AZ-628 | 2a | 0.12 | >3.8 (5 μM) | II |

¹Measured by luciferase tagged HCV GT-2a cell culture derived virus in 50% confluent cells at MOI 0.02.
²Ratio of EC₅₀and CC₅₀, where EC₅₀ is the concentration necessary to inhibit viral reproduction by 50% and CC₅₀ is the intrinsic drug cytotoxicity measured in the absence of virus. Number in parentheses indicates the highest concentration tested in the experiment. > sign indicates that concentration range tested was too low to fully evaluate the TI, so the lower limit is specified.
³Defined by the specificity of the inhibitor to a RAF isoform

### Example 4

### Synergistic Antiviral Potency against HCVcc

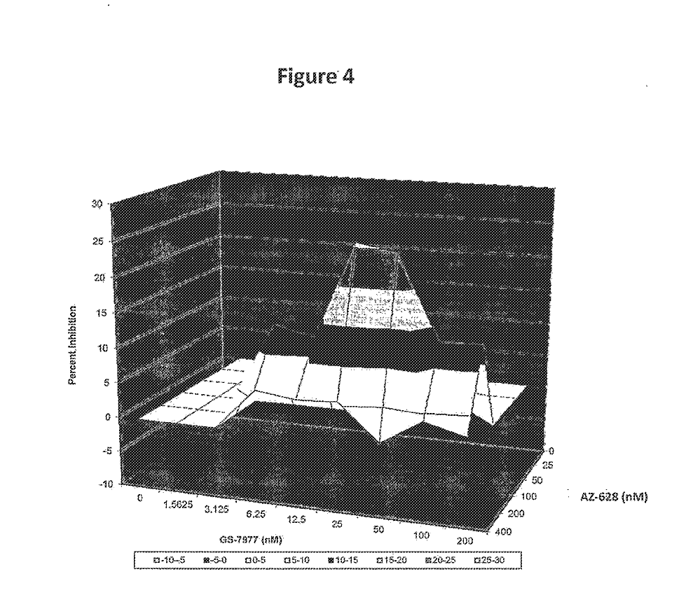

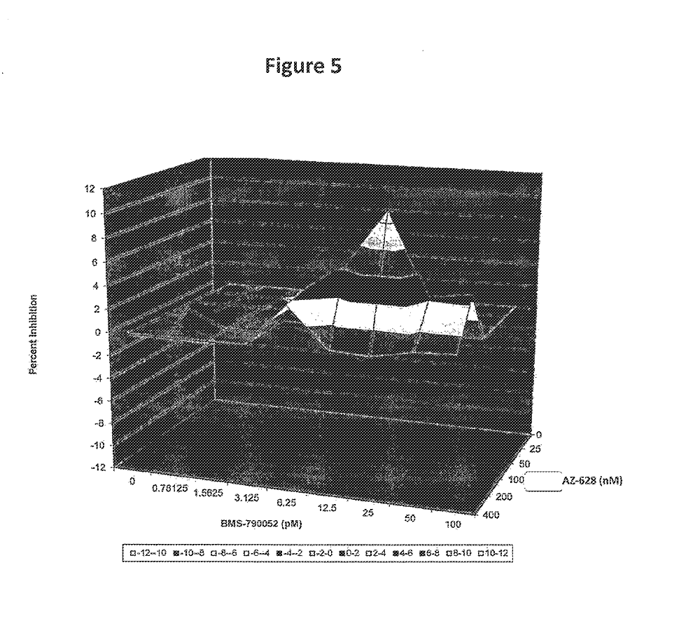

The tested RAF inhibitors were evaluated for an ability to synergize with other current, late stage or marketed direct-acting antivirus (DAAs). Synergistic antiviral potency was measured with AZ-628 and the direct-acting antiviral GS7977/Sobosuvir, a nucleotide analog that inhibits the viral RNA-dependent RNA polymerase of HCV or with AZ-628 and the direct-acting antiviral BMS-790052/Daclatasvir, an inhibitor of the non-structural protein NS5a, which plays a vital role in HCV genomic replication in the host. The top panels of Tables 4 and 5 show the % antiviral inhibition as a function of concentration of both drugs. The bottom panels of Tables 4 and 5 show the synergy volume calculated from the top table at the 95% confidence interval. The three-dimensional plots of FIGS. 4 and 5 display the synergy volume data at the 95% confidence interval for AZ-628 in combination with GS7977 and BMS-790052, respectively. Table 6 compares the single drug and synergy drug EC₅₀ values tested in HCVcc GT2a. The results of these tests indicate that DAAs that are in late stage development synergize well with AZ-628 and this may have further applications in a combination therapy for treating HCV infection.

TABLE 4
Synergistic antiviral potency measured with AZ-628 and GS7977

| | | GS-7977 (nm) | | | | | | | | | Antiviral |
|---|---|---|---|---|---|---|---|---|---|---|---|
| | [DRUG] | 0 | 1.563 | 3.125 | 6.25 | 12.5 | 25 | 50 | 100 | 200 | Potency |
| IkT-064 | 400 | 73.43 | 73.89 | 74.28 | 81.68 | 81.78 | 84.14 | 83.25 | 84.96 | 90.2 | |
| (nM) | 200 | 5.15 | 8.85 | 4.46 | 24.83 | 27.78 | 37.33 | 47.26 | 48.43 | 63.86 | |
| | 100 | 0 | 0 | 0 | 0 | 0 | 5.55 | 13.51 | 26.5 | 50.66 | |
| | 50 | 0 | 0 | 0 | 0 | 0 | 1.62 | 11.55 | 22.74 | 47.11 | |
| | 25 | 0 | 0 | 0 | 0 | 0 | 2.29 | 9.57 | 22.42 | 45.09 | |
| | 0 | 0 | 0 | 0 | 0 | 0 | 2.65 | 11.54 | 21.51 | 44.74 | |
| | | GS-7977 (nm) | | | | | | | | | Synergy |
| | [DRUG] | 0 | 1.563 | 3.125 | 6.25 | 12.5 | 25 | 50 | 100 | 200 | Plot (95%) |
| IkT-064 | 400 | 0 | 0 | 0 | 5.597 | 5.234 | 5.541 | 0.458 | 4.085 | 2.587 | Synergy |
| (nM) | 200 | 0 | 0 | 0 | 13.35 | 12.09 | 25.67 | 24.71 | 12.88 | 13.09 | Volume |
| | 100 | 0 | 0 | 0 | 0 | 0 | 0 | 0 | 3.05 | 0 | 128.71 |
| | 50 | 0 | 0 | 0 | 0 | 0 | 0 | 0 | 0 | 0 | Antagonism |
| | 25 | 0 | 0 | 0 | 0 | 0 | 0 | 0 | 0 | 0 | Volume |
| | 0 | 0 | 0 | 0 | 0 | 0 | 0 | 0 | 0 | 0 | 0 |

TABLE 5
Synergistic antiviral potency measured with AZ-628 and the direct-acting antiviral BMS-790052

| | | BMS-790052 (pM) | | | | | | | | | Antiviral |
|---|---|---|---|---|---|---|---|---|---|---|---|
| | [DRUG] | 0 | 0.781 | 1.563 | 3.125 | 6.25 | 12.5 | 25 | 50 | 100 | Potency |
| IkT-064 | 400 | 74.25 | 77.01 | 75.45 | 80.19 | 81.83 | 83.5 | 83.44 | 87.37 | 91.74 | |
| (nM) | 200 | 17.39 | 21.94 | 18.29 | 34.05 | 36.01 | 44.17 | 47.45 | 51.76 | 73.6 | |
| | 100 | 0 | 0 | 0 | 0.89 | 9.67 | 9.33 | 15.83 | 33.9 | 63.67 | |
| | 50 | 0 | 0 | 0 | 0 | 6.13 | 7.23 | 20.08 | 32.34 | 63.34 | |
| | 25 | 2.83 | 0 | 0 | 4.97 | 2.98 | 9.29 | 19.07 | 28.92 | 61.71 | |
| | 0 | 0 | 0 | 0 | 8.7 | 8.72 | 17.27 | 19.54 | 33.86 | 59.54 | |
| | | BMS-790052 (pM) | | | | | | | | | Synergy |
| | [DRUG] | 0 | 0.781 | 1.563 | 3.125 | 6.25 | 12.5 | 25 | 50 | 100 | Plot (95%) |
| IkT-064 | 400 | 0 | 0 | 0 | 0 | 3.664 | 0.194 | 0 | 0.696 | 0.827 | Synergy |
| (nM) | 200 | 0 | 1.532 | 0 | 0 | 4.011 | 6.414 | 10.94 | 4.205 | 4.648 | Volume |
| | 100 | 0 | 0 | 0 | 0 | 0 | −2.766 | 0 | 0 | 0 | 37.13 |
| | 50 | 0 | 0 | 0 | 0 | 0 | 0 | 0 | 0 | 0 | Antagonism |
| | 25 | 0 | 0 | 0 | −0.43 | −3.498 | 0 | 0 | 0 | 0 | Volume |
| | 0 | 0 | 0 | 0 | 0 | 0 | 0 | 0 | 0 | 0 | −6.69 |

TABLE 6
Synergistic antiviral potency against HCVcc of AZ-628 and DAAs

| | | | EC50 | EC50 | |
|---|---|---|---|---|---|
| | | EC50 | AZ-628 + | AZ-628 + | Fold |
| Drug | Genotype | alone | sofosbuvir | daclatasvir | Synergy |
| Sofosbuvir | 2a | 250 nM | 50 nM | — | 5 |
| (GS-7977) | | | | | |
| (NS5b inhibitor, | | | | | |
| ‘nuc’) | | | | | |
| Daclatasvir | 2a | 0.1 nM | — | 0.033 nM | 3 |
| (BMS-790052 | | | | | |
| (NS5a inhibitor) | | | | | |
| AZ-628 | 2a | 300 nM | 200 nM | 200 nM | — |

1. A method of treating a viral infection, such as an HCV infection, in a subject in need thereof, comprising administering to the subject an effective amount of a RAF kinase inhibitor or a prodrug thereof.

2. The method of claim 1, wherein the RAF kinase inhibitor is a compound having a structure of formula: 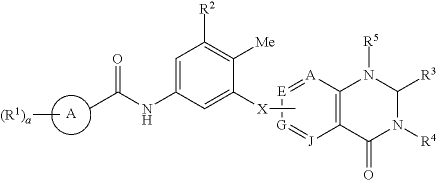
wherein:
Ring A is carbocyclyl or heterocyclyl; wherein if said heterocyclyl contains an —NH-moiety that nitrogen may be optionally substituted by a group selected from R⁶;
R¹ is a substituent on carbon and is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆6alkyl)₂carbamoyl, C₁₋₆alkylS(O)ₐ wherein a is 0 to 2, C₁₋₆alkoxycarbonyl; N-(C₁₋₆alkyl)sulphamoyl, N,N-(C₁₋₆alkyl)2sulphamoyl, N-(C₁₋₆alkoxy)sulphamoyl, N-(C₁₋₆alkyl)-N-(C₁₋₆alkoxy)sulphamoyl, C₁₋₆alkylsulphonylamino, carbocyclyl-R⁷- or heterocyclyl-R⁸-; wherein R¹ may be optionally substituted on carbon by one or more R⁹; and wherein if said heterocyclyl contains an —NH-moiety that nitrogen may be optionally substituted by a group selected from R¹⁰;
n is selected from 0-4; wherein the values of R¹ may be the same or different;
R2 is selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₂₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl, N,N-(C₁₋₆alkyl)₂sulphamoyl, C₁₋₆alkylsulphonylamino, carbocyclyl-R¹¹- or heterocyclyl-R¹²-; wherein R² may be optionally substituted on carbon by one or more R¹³; and wherein if said heterocyclyl contains an —NH— moiety that nitrogen may be optionally substituted by a group selected from R14;
X is NR¹⁵ or O;
one of A, E, G and J is C which is attached to X of the formula; the other three are independently selected from CR¹⁶ or N;
R³ and R¹⁶ are independently selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, Cvalkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbarmoyl, C₁₋₆alkylS(O)ₐ wherein a is 0 to 2, C₁₋₆alkoxycarbonyl, N-(C₁₋₆alkyl)sulphamoyl, N,N-(C₁₋₆alkyl)₂sulphamoyl, C₁₋₆alkylsulphonylamino, carbocyclyl-R¹⁷- or heterocyclyl-R¹ wherein R³ and R¹⁶ independently of each other may be optionally substituted on carbon by one or more R¹⁹; and wherein if said heterocyclyl contains an —NH— moiety that nitrogen may be optionally substituted by a group selected from R²⁰;
R⁴, R⁵ and R¹⁵ are independently selected from hydrogen, C₁₋₆alkyl, C₁₋₆alkylsulphonyl, C₁₋₆alkoxycarbonyl, carbamoyl, carbocyclyl, heterocyclyl, N-(C₁₋₆alkyl)carbamoyl and N,N-(C₁₋₆alkyl)carbamoyl; wherein R⁴, R⁵ and R¹⁵ independently of each other may be optionally substituted on carbon by one or more R²¹;
the bond “ [symbol] ” between the —NR⁵— and —CR³— of the formula is either (i) a single bond wherein R⁵ is as defined above, or (ii) a double bond wherein R⁵ is absent;
R⁹, R¹³, R¹⁹ and R²¹ are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆lkyl)₂carbamoyl, C₁₋₆alkylS(O)ₐ wherein a is 0 to 2, C₁₋₆alkoxycarbonyl, C₁₋₆alkoxycarbonylamino, N-(C₁₋₆lkyl)sulphamoyl, N,N-(C₁₋₆alkyl)₂sulphamoyl, C₁₋₆alkylsulphonylamino, carbocyclyl-R²²- or heterocyclyl-R²³-; wherein R⁹, R¹³, R¹⁹ and R²¹ independently of each other may be optionally substituted on carbon by one or more R²⁴; and wherein if said heterocyclyl contains an —NH— moiety that nitrogen may be optionally substituted by a group selected from R²⁵;
R⁷, R⁸, R¹², R¹⁷, R¹⁸, R²² and R²³ are independently selected from a direct bond, —O—, —N(R²⁶)—, —C(O)—, —N(R²⁷)C(O)—, —C(O)N(R²⁸)—, —S(O)ₛ—, —SO₂N(R²⁹)— or —N(R³⁰)SO₂—; wherein R²⁶, R²⁷, R²⁸, R²⁹ and R³⁰ is hydrogen, C₁₋₆alkoxycarbonyl or C₁₋₆alkyl and s is 0-2;
R⁶, R¹⁰, R¹⁴, R²⁰ and R²⁵ are independently selected from C₁₋₆alkyl, C₁₋₆alkylsulphonyl, C₁₋₆alkoxycarbonyl, carbamoyl, N-(C₁₋₆alkypearbamoyl, N,N-(C₁₋₆alkyl)carbamoyl, benzyl, benzyloxycarbonyl, benzoyl and phenylsulphonyl;
R²⁴ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N-dimemylsulphamoyl, N,N-diethylsulphamoyl or N-methyl-N-ethylsulphamoyl;
or a pharmaceutically acceptable salt thereof.

3. The method of claim 2, wherein the RAF inhibitor is AZ-628: 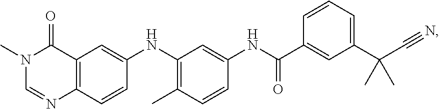 [SMILES: Cc1ccc(NC(=O)c2cccc(C(C)(C)C#N)c2)cc1Nc1ccc2ncn(C)c(=O)c2c1]
or a pharmaceutically acceptable salt thereof.

4. The method of claim 1, wherein the RAF kinase inhibitor is a compound having a structure of formula:
A-D-B
wherein:
D is —NH—C(O) —NH—,
A is a substituted moiety of up to 40 carbon atoms of the formula: -L-(M-L¹)_{q}, where L is a 5 or 6 membered cyclic structure bound directly to D, L¹ comprises a substituted cyclic moiety having at least 5 members, M is a bridging group having at least one atom, q is an integer of from 1-3; and each cyclic structure of L and L¹ contains 0-4 members of the group consisting of nitrogen, oxygen and sulfur, and
B is a substituted or unsubstituted, up to tricyclic aryl or heteroaryl moiety of up to 30 carbon atoms with at least one 6-member cyclic structure bound directly to D containing 0-4 members of the group consisting of nitrogen, oxygen and sulfur,
wherein L¹ is substituted by at least one substituent selected from the group consisting of —SO₂Rₓ, —C(O) Rₓ and —C(NR_{y})R_{z},
R_{y} is hydrogen or a carbon based moiety of up to 24 carbon atoms optionally containing heteroatoms selected from N, S and O and optionally halosubstituted, up to per halo,
R_{z} is hydrogen or a carbon based moiety of up to 30 carbon atoms optionally containing heteroatoms selected from N, S and O and optionally substituted by halogen, hydroxy and carbon based substituents of up to 24 carbon atoms, which optionally contain heteroatoms selected from N, S and O and are optionally substituted by halogen;
Rₓ is R_{z} or NRₐR_{b} where Rₐ and R_{b} are
a) independently hydrogen,
a carbon based moiety of up to 30 carbon atoms optionally containing heteroatoms selected from N, S and O and optionally substituted by halogen, hydroxy and carbon based substituents of up to 24 carbon atoms, which optionally contain heteroatoms selected from N, S and O and are optionally substituted by halogen, or
—OSi(R_{f})₃ where R_{f} is hydrogen or a carbon based moiety of up to 24 carbon atoms optionally containing heteroatoms selected from N, S and O and optionally substituted by halogen, hydroxy and carbon based substituents of up to 24 carbon atoms, which optionally contain heteroatoms selected from N, S and O and are optionally substituted by halogen; or
b) Rₐ and R_{b} together form a 5-7 member heterocyclic structure of 1-3 heteroatoms selected from N, S and O, or a substituted 5-7 member heterocyclic structure of 1-3 heteroatoms selected from N, S and O substituted by halogen, hydroxy or carbon based substituents of up to 24 carbon atoms, which optionally contain heteroatoms selected from N, S and O and are optionally substituted by halogen; or
c) one of Rₐ or R_{b} is —C(O)—, a C₁-C₅ divalent alkylene group or a substituted C₁-C₅ divalent alkylene group bound to the moiety L to form a cyclic structure with at least 5 members, wherein the substituents of the substituted C₁-C₅ divalent alkylene group are selected from the group consisting of halogen, hydroxyl, and carbon based substituents of up to 24 carbon atoms, which optionally contain heteroatoms selected from N, S and O and are optionally substituted by halogen;
where B is substituted, L is substituted of L¹ is additionally substituted, the substituents are selected from the group consisting of halogen, up to per-halo, and Wn, where n is 0-3;
wherein each W is independently selected from the group consisting of —CN, —CO₂R⁷, —C(O)NR⁷R⁷, —C(O)—R⁷, —NO₂, —OR⁷, —SR⁷, —NR⁷R⁷, —NR⁷C(O)OR⁷, —NR⁷C(O)R⁷, —Q—Ar, and carbon based moieties of up to 24 carbon atoms, optionally containing heteroatoms selected from N, S and O and optionally substituted by one or more substituents independently selected from the group consisting of —CN, —CO₂R⁷, —C(O)R⁷, —C(O)NR⁷R⁷, —OR⁷, —SR⁷, —NR⁷R⁷, —NO₂, —NR⁷C(O)R⁷, —NR⁷C(O) OR⁷ and halogen up to per-halo; with each R⁷ independently selected from H or a carbon based moiety of up to 24 atoms, optionally containing heteroatoms selected from N, S and O and optionally substituted by halogen,
wherein Q is —O—, —S—, —N(R⁷)—, —(CH²)ₘ—, —C(O)—, —CH(OH)—, —(CH₂)ₘO—, —(CH₂)ₘS—, —(CH₂)ₘN(R⁷)—, —O(CH₂)ₘ—CHX^{a}—, —CX^{a}₂—, —S—(CH₂)ₘ— and —N(R₇)(CH₂)ₘ—, where m=1−3, and X^{a} is halogen; and
Ar is a 5- or 6-member aromatic structure containing 0-2 members selected from the group consisting of nitrogen, oxygen and sulfur, which is optionally substituted by halogen, up to per-halo, and optionally substituted by Zₙ₁, wherein n1 is 0 to 3 and each Z is independently selected from the group consisting of —CN, —CO₂R⁷, —C(O)R⁷, —C(O)NR⁷R⁷, —NO₂,
—OR⁷, —SR⁷—NR⁷R⁷, —NR⁷C(O)OR⁷, —NR⁷C(O)R⁷, and a carbon based moiety of up to 24 carbon atoms, optionally containing heteroatoms selected from N, S and O and optionally substituted by one or more substituents selected from the group consisting of —CN, —CO₂R⁷,
—COR⁷, —C(O)NR⁷R⁷, —OR⁷, —SR⁷, —NO₂, —NR⁷R⁷, —NR⁷C(O)R⁷, and —NR⁷C(O)OR⁷, with R⁷ as defined above.

5. The method of claim 4, wherein the RAF inhibitor is sorafenib: 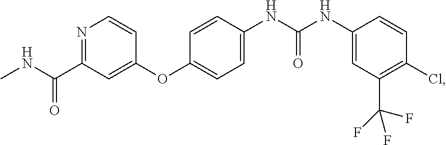 [SMILES: CNC(=O)c1cc(Oc2ccc(NC(=O)Nc3ccc(Cl)c(C(F)(F)F)c3)cc2)ccn1]
or a pharmaceutically acceptable salt thereof.

6. The method of claim 4, wherein the RAF inhibitor is regorafenib: 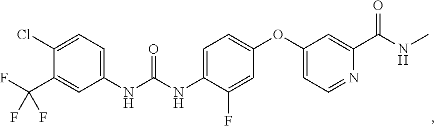 [SMILES: CNC(=O)c1cc(Oc2ccc(NC(=O)Nc3ccc(Cl)c(C(F)(F)F)c3)c(F)c2)ccn1]
or a pharmaceutically acceptable salt thereof.

7. The method of claim 1, wherein the RAF kinase inhibitor is a compound having a structure of formula: 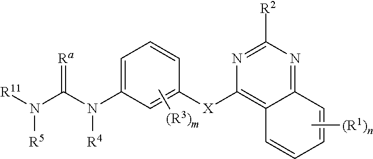
or a pharmaceutically acceptable salt or prodrug thereof, wherein
X is O or S(O)ₜ;
R^{a} is O or S;
each R¹ is independently selected from (i) or (ii) below:
(i) each R¹ is independently selected from a group consisting of halo, nitro, alkyl, alkenyl, alkynyl, cycloalkyl, cycloalkylalkyl, heterocyclyl, heterocyclylalkyl, aryl, aralkyl, heteroaryl, heteroaralkyl, —R⁶OR⁷, —R⁶SR⁷, —R⁶S(O)ₜR⁸, —R⁶N(R⁷)₂, —R⁶OR⁹OR⁷, —R⁶OR⁹SR⁷, —R⁶OR⁹S(O)ₜR⁸, —R⁶OR⁹S(O)ₜN(R⁷)₂, —R⁶OR⁹N(R⁷)₂, —R⁶SR⁹OR⁷, —R⁶SR⁹SR⁷, —R⁶SR⁹N(R⁷)₂, —R⁶N(R⁷)R⁹N(R⁷)₂, —R⁶N(R⁷)R⁹OR⁷, —R⁶N(R⁷)R⁹SR⁷, —R⁶CN, —R⁶C(O)R⁷, —R⁶C(O)OR⁷, —R⁶C(O)OR⁹OR⁷, —R⁶C(O)N(R⁷)₂, —R⁶C(O)N(R⁷)OR⁷, —R⁶C(NR⁷)N(R⁷)₂, —R⁶C(O)N(R⁷)R⁹N(R⁷)₂, —R⁶C(O)N(R⁷)R⁹OR⁷, —R⁶C(O)N(R⁷)R⁹SR⁷, —R⁶C(O)SR⁸, —R⁶S(O)ₜOR⁷, —R⁶S(O)ₜN(R⁷)₂, —R⁶S(O)ₜN(R⁷)N(R⁷)₂, —R⁶S(O)ₜN(R⁷)N═C(R⁷)₂, —R⁶S(O)ₜN(R)⁷)C(O)R⁸, —R⁶S(O)ₜN(R⁷)C(O)N(R⁷)₂, —R⁶S(O)ₜN(R⁷)C(NR⁷)N(R⁷)₂, —R⁶N(R⁷)C(O)R⁸, —R⁶N(R⁷)C(O)OR⁸, —R⁶N(R⁷)C(O)N(R⁷)₂, —R⁶N(R⁷)C(NR⁷)N(R⁷)₂, —R⁶N(R⁷)C(S)N(R⁷)₂, and —R⁶N(R⁷)S(O)ₜR⁸, or
(ii) any two adjacent R¹ groups together form an alkylenedioxy group; each R⁶ is independently a direct bond, alkylene chain or alkenylene chain; each R⁷ is independently selected from (i) or (ii) below:
(i) each R⁷ is selected from a group consisting of hydrogen, alkyl, alkenyl, alkynyl, cycloalkyl, cycloalkylalkyl, aryl, aralkyl, heterocyclyl, heterocyclylalkyl, heteroaryl and heteroaralkyl, or
(ii) two R⁷ groups together with the N atom to which they are attached form a heterocyclyl or heteroaryl;
each R⁸ is independently selected from a group consisting of alkyl, alkenyl, alkynyl, cycloalkyl, cycloalkylalkyl, aryl, aralkyl, heterocyclyl, heterocyclylalkyl, heteroaryl and heteroaralkyl;
each R⁹ is independently an alkylene chain or an alkenylene chain;
R² is hydrogen, halo, alkyl, amino or alkylamino;
R³ is halo or alkyl;
R⁴ and R⁵ are selected as follows:
a) R⁴ and R⁵ are each independently hydrogen or alkyl, or
b) R⁴ and R⁵, together with the N atom to which they are attached, form an oxo-substituted heterocyclyl;
R¹¹ is aryl, heteroaryl or heterocyclyl;
m is an integer from 0 to 4;
n is an integer from 0 to 4;
t is an integer from 0 to 2;
R¹, R², R³, R⁴, R⁵, R⁶, R⁷, R⁸, R⁹ and R¹¹ are optionally substituted with one, two or three substituents independently selected from Q¹, wherein Q¹ is nitro, halo, azido, cyano, oxo, thioxo, imino, alkyl, alkenyl, alkynyl, cycloalkyl, cycloalkylalkyl, aryl, aralkyl, heteroaryl, heteroaralkyl, heterocyclyl, heterocyclylalkyl, —R^{u}OR^{x}, —R^{u}OR^{u}OR^{x}, —R^{u}OR^{u}N(R^{y})(R^{z}), —R^{u}N(R^{y})(R^{z}), —R^{u} SR^{x}, —R^{u} C(J)R^{x}, —R^{u}C(J)OR^{x}, —R^{u}C(J)N(R^{y})(R^{z}), —R^{u}C(J)SR^{x}, —R^{u}S(O)^{t}R^{w}, —R^{u}OC(J)R^{x}, —R^{u}OC(J)OR^{x}, —R^{u}OC(J)N(R^{y})(R^{z}), —R^{u}OC(J)SR^{x}, —R^{u} N(R^{x})C(J)R^{x}, —R^{u}N(R^{x})C(J)OR^{x}, —R^{u}N(R^{x})C(J)N(R^{y})(R^{z}), —R^{u}N(R^{x})C(J)SR^{x}, —R^{u}Si(R^{w})₃, —R^{u}N(R^{x})S(O)ₜR^{w}, —R^{u}N(R^{x}) R^{u}S(O)₂R^{w}, —R^{u}N(R^{x})S(O)₂N(R^{y})(R^{z}), —R^{u}S(O)₂N(R^{y})(R^{z}), —R^{u}P(O)(R^{v})₂, —R^{u}OP(O)(R^{v})₂, —R^{u}C(J)N(R^{x})S(O)₂R^{w}, —R^{u}C(J)N(R^{x})N(R^{x})S(O)₂R^{w}, —R^{u}C(R^{x})═N(OR^{x}) and —R^{u}C(R^{x})═NN(R^{y})(R^{z});
when Q¹ is alkyl, alkenyl or alkynyl, each Q¹ is optionally substituted with halo, cyano, hydroxy or alkoxy;
when Q¹ is cycloalkyl, cycloalkylalkyl, aryl, aralkyl, heteroaryl, heteroaralkyl, heterocyclyl, or heterocyclylalkyl, each Q¹ is optionally substituted with halo, alkyl, haloalkyl, hydroxyalkyl, alkoxyalkyl, cyanoalkyl, alkoxy or hydroxyl;
each R^{u} is independently alkylene or a direct bond;
each R^{v} is independently alkyl, alkenyl, alkynyl, cycloalkyl, cycloalkylalkyl, heterocyclyl, heterocyclylalkyl, aryl, aralkyl, heteroaryl, heteroaralkyl, hydroxy,—OR^{x} or —N(R^{y})(R^{z});
R^{w} is alkyl, alkenyl, alkynyl, cycloalkyl, cycloalkylalkyl, heterocyclyl, heterocyclylalkyl, aryl, aralkyl, heteroaryl, or heteroaralkyl;
each R^{x} is independently hydrogen, alkyl, alkenyl, alkynyl, cycloalkyl, cycloalkylalkyl, heterocyclyl, heterocyclylalkyl, aryl, aralkyl, heteroaryl, or heteroaralkyl;
R^{y} and R^{z} are each independently hydrogen, alkyl, alkenyl, alkynyl, cycloalkyl, cycloalkylalkyl, heterocyclyl, heterocyclylalkyl, aryl, aralkyl, heteroaryl, or heteroaralkyl;
R^{Y} and R^{z}, together with the nitrogen atom to which they are attached, form a heterocyclyl or heteroaryl; and
J is O, NR^{X} or S.

8. The method of claim 7, wherein the RAF kinase inhibitor is CEP32496: 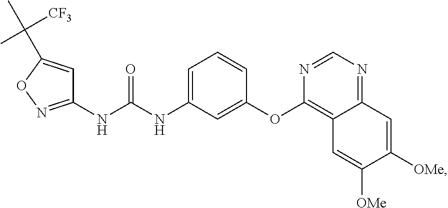 [SMILES: COc1cc2ncnc(Oc3cccc(NC(=O)Nc4cc(C(C)(C)C(F)(F)F)on4)c3)c2cc1OC]
or a pharmaceutically acceptable salt thereof.

9. The method of claim 1, wherein the RAF kinase inhibitor is a compound having a structure of formula: 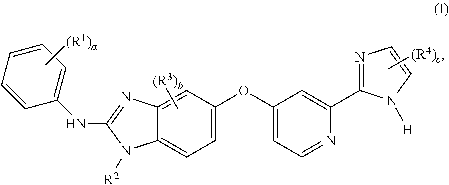
wherein each R¹ is independently selected from hydroxy, halo, C₁₋₆alkyl, C₁₋₆alkoxy, (C₁₋₆alkyl)sulfanyl, (C₁₋₆alkyl)sulfonyl, cycloalkyl, heterocycloalkyl, phenyl and heteroaryl;
R² is C₁₋₆alkyl or halo(C₁₋₆alkyl);
each R³ is independently selected from halo, C₁₋₆alkyl and C₁₋₆alkoxy;
each R⁴ is independently selected from hydroxy, C₁₋₆alkyl, C₁₋₆alkoxy, halo, heterocycloalkylcarbonyl, carboxyl, (C₁₋₆alkoxy)carbonyl, aminocarbonyl, C₁₋₆alkylaminocarbonyl, carbonitrile, cycloalkyl, heterocycloalkyl, phenyl and heteroaryl;
wherein R¹, R², R³ and R⁴ may be optionally substituted with one or more substituents independently selected from hydroxy, halo, C₁₋₆alkyl, halo(C₁₋₆alkyl), C₁₋₆alkoxy and halo(C₁₋₆alkoxy);
a is 1, 2, 3, 4 or 5;
b is 0, 1, 2 or 3; and
c is 1 or 2;
or a tautomer, stereoisomer, polymorph, ester, metabolite, or prodrug thereof or a pharmaceutically acceptable salt of the compound, tautomer, stereoisomer, polymorph, ester, metabolite or prodrug.

10. The method of claim 9, wherein the RAF inhibitor is Raf-265: 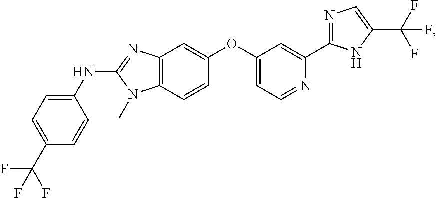 [SMILES: Cn1c(Nc2ccc(C(F)(F)F)cc2)nc2cc(Oc3ccnc(-c4ncc(C(F)(F)F)[nH]4)c3)ccc21]
or a pharmaceutically acceptable salt thereof.

11. The method of claim 1, wherein the RAF kinase inhibitor is a compound having a structure of formula: 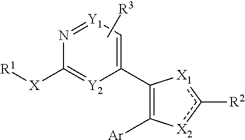
wherein
X is O, CH₂, CO, S or NH, or the moiety X-R¹ is hydrogen;
Y₁ and Y₂ are independently N or CH;
R¹ is hydrogen, C₁₋₆alkyl, C₃₋₇cycloalkyl, aryl, arylC₁₋₆alkyl, heterocyclyl, heterocyclylC₁₋₆alkyl, heteroaryl, or heteroarylC₁₋₆alkyl, any of which may be optionally substituted; in addition when X is CH² then R¹ may be hydroxyl or C₁₋₆alkoxy which may be optionally substituted;
R² is H, C₁₋₆alkyl, C₂₋₆alkenyl, C₃₋₇cycloalkyl, C₅₋₇cycloalkenyl, heterocyclyl, aryl or heteroaryl, any of which may be optionally substituted;
Ar is a group of the formula a) or b): 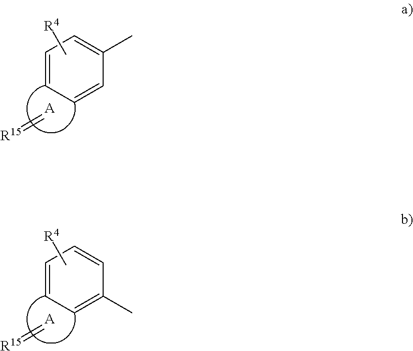
wherein A represents a fused 5- to 7-membered ring optionally containing up to two heteroatoms selected from O, S and NR⁵, wherein R⁵ is hydrogen or C₁₋₆alkyl, which ring is optionally substituted by up to 2 substituents selected from halogen, C₁₋₆alkyl, hydroxy, C₁₋₆alkoxy or keto;
R³ and R⁴ are independently selected from hydrogen, halogen, C₁₋₆alkyl, aryl, aryl C₁₋₆alkyl, C₁₋₆alkoxy, C₁₋₆alkoxyC₁₋₆alkyl, haloC₁₋₆alkyl, arylC₁₋₆alkoxy, hydroxy, nitro, cyano, azido, amino, mono- and di-N-C₁₋₆alkylamino, acylamino, arylcarbonylamino, acyloxy, carboxy, carboxy salts, carboxy esters, carbamoyl, mono- and di-N-C₁₋₆alkylcarbamoyl, C₁₋₆alkoxycarbonyl, aryloxycarbonyl, ureido, guanidino, C₁₋₆alkylguanidino, amidino, C₁₋₆alkylamidino, sulphonylamino, aminosulphonyl, C₁₋₆alkylthio, C₁₋₆alkylsulphinyl or C₁₋₆alkylsulphonyl;
R¹⁵is O or N—OH;
one of X₁ and X₂ is N and the other is NR⁶ wherein R⁶ is hydrogen or C₁₋₆alkyl; or a pharmaceutically acceptable salt thereof.

12. The method of claim 11, wherein the RAF kinase inhibitor is SB-590885: 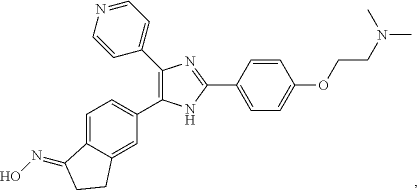 [SMILES: CN(C)CCOc1ccc(-c2nc(-c3ccncc3)c(-c3ccc4c(c3)CC/C4=N\O)[nH]2)cc1]
or a pharmaceutically acceptable salt thereof.

13. The method of claim 1, wherein the RAF inhibior is administered conjointly with at least one additional therapeutic agent.

14. The method of claim 13, wherein said at least one additional therapeutic agent is an antival agent selected from HCV protease inhibitors, HCV polymerase inhibitors, agents targeting host cell activities involved in HCV replication and inosine monophosphate dehydrogenase (IMPDH) inhibitors.

15. The method of claim 14, wherein the at least one additional therapeutic agent is GS-7977: 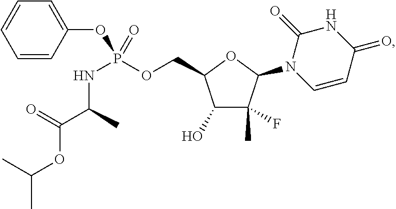 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]
or a pharmaceutically acceptable salt thereof.

16. The method of claim 14, wherein the at least one additional therapeutic agent is BMS-790052: 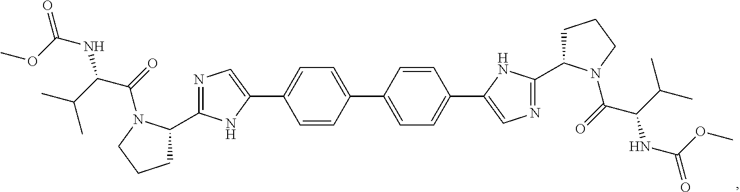
or a pharmaceutically acceptable salt thereof.
